(19) United States
(12) Patent Application Publication
(10) Pub. No.: US 2026/0193687 A1
(43) Pub. Date: Jul. 9, 2026

(54) TWO-STRAIN SYSTEM FOR PRODUCING OLIGOSACCHARIDES

(71) Applicant: DSM IP Assets B.V., Maastricht (NL)

(72) Inventors: Manos PAPADAKIS, Hørsholm (DK); Ted JOHANSON, Hørsholm (DK); Getachew S. MOLLA, Hørsholm (DK); Niels BANKE, Hørsholm (DK)

(21) Appl. No.: 19/132,304

(22) PCT Filed: Nov. 27, 2023
(86) PCT No.: PCT/EP2023/083121

§ 371 Date: May 22, 2025

(30) Foreign Application Priority Data
Nov. 25, 2022 (EP) 22209673.7
Nov. 25, 2022 (EP) 22209675.2
Aug. 25, 2023 (EP) 23193552.9

(51) Int. Cl. C12P 19/04; C12N 1/20; C12N 9/10; C12N 9/24; C12P 19/14; C12P 19/18; C12R 1/19

(52) U.S. Cl. CPC C12P 19/04; C12N 1/20; C12N 9/1081; C12N 9/2402; C12P 19/14; C12P 19/18; C12R 2001/19; C12Y 204/99004; C12Y 302/01018

(57) ABSTRACT

The present disclosure relates to a method for producing one or more oligosaccharides by co-culturing two different oligosaccharide producing strains. The method is useful for producing a balanced mixture of oligosaccharides, a selected oligosaccharide with reduced by-product formation and complex oligosaccharides, in particular complex fucosylated and sialylated HMOs. The co-cultivation is controlled by controlling the carbon source the individual strains can grow on.

### FIELD OF INVENTION

The present disclosure relates to a method for producing one or more oligosaccharides by co-culturing two different oligosaccharide producing strains. The method is useful for producing a balanced mixture of oligosaccharides, a selected oligosaccharide with reduced by-product formation and complex oligosaccharides, in particular complex fucosylated and sialylated HMOs, but also neutral oligosaccharides with six monosaccharide units or more. The co-cultivation is controlled by controlling the carbon source the individual strains can grow on.

### BACKGROUND

Production of human milk oligosaccharides (HMOs) have been pursued via chemical synthetic routes, enzymatic routes and in vivo fermentation approaches. For industrial purposes the chemical routes are too complex and expensive. Due to the nature of enzymatic reactions the HMOs produced by the enzymatic route will always be a mixture of the donor, the acceptor and the third oligosaccharide (HMO) as well as a side-product released from the donor substrate (the leaving group e.g., lactose) (see for example WO2012/156897, WO2012/156898 and WO2016/063262).

Bioproduction systems using in vivo fermentation of the HMOs is currently the preferred mode of production for the smaller fucosylated and sialylated and neutral core HMOs (for review see Bych et al 2019, Current Opinion in Biotechnology 56:130-137). In production cells where more than one glycosidase activity is needed to produce the HMO product of interest, by-product HMOs may also be present at the end of fermentation. If a mixture of HMOs is desired, it is common to produce the two HMOs of interest in two separate fermentations, which are subjected to individual purification streams and blend the two or more purified HMOs.

WO 2015/032413 describes a method for producing more complex HMOs of at least four monosaccharide units, by exogenously adding an acceptor molecule to a culture where a cell is capable internalizing the acceptor to produce the HMO of at least for monosaccharide units.

For most fermentation processes lactose is used as the initial substrate. WO 2015/150328 describes a process where instead of adding lactose to the medium a cell is modified to produce lactose which the cell then glycosylates further to produce an HMO, as an alternative it is mentioned that one cell can produce the lactose and another cell can internalize the lactose and produce the HMO. It is not specified that the cells are grow in the same culture nor that they grow on different carbon sources.

WO 2015/036138 describes the use of a first strain to produce the desired oligosaccharide and a second strain expressing a glycosidase to remove by-products produced by the first strain. The setup is depicted as a continuous fermentation set-up with two separate fermenters. Similarly, WO 2022/242860 describes a sequential fermentative production of oligosaccharides in two separated compartments for cultivating two different genetically engineered microbial cells separated by a semipermeable membrane.

### OBJECTIVE OF THE INVENTION

The present disclosure has identified that it is possible to control a co-culture of two-strains producing two different oligosaccharides by controlling the carbon source the individual strains can grow on.

The ability to co-culture two strains producing two different oligosaccharides can be applied in multiple ways
i) the co-cultured strains can produce a balanced mixture of the oligosaccharides produced by each cell, where the amount of the individual oligosaccharide can be controlled by controlling the amount of the different carbon sources. An advantage of this system is that it can save production capacity since a mixture of two HMOs can be produced in a single fermentation instead of having to produce the oligosaccharides separately and blend them after individual fermentation and purification.
ii) the co-cultured strains can produce an oligosaccharide, such as an HMO, with reduced oligosaccharide by-products. Here the first strain produces a first oligosaccharide serving as an intermediate oligosaccharide, which is taken up by the second strain which use the internalized intermediate oligosaccharide as substrate to generate a second oligosaccharide (illustrated in FIG. 1). An advantage of this system is that the formation of oligosaccharide by-products that are usually encountered in single cell systems can be reduced by separating two or more glycosyltransferases in two different cells. The manner that these glycosyltransferases are separated among the two cells depends on the desired HMO outcome/product. Furthermore, separating the glycosyltransferase reactions into two different cells allows for direct control of their respective rates by adjusting the sugar addition profile, which can be used to balance the respective rates to control the by-product formation.
iii) the co-cultured strains can be used to produce a complex oligosaccharide by reacting the oligosaccharides produced by each cell with a transglycosidase in the culture medium to form a third complex oligosaccharide (illustrated in FIG. 2). This application of the two-strain system is also termed the hybrid two-strain system. An advantage of this system is that complex oligosaccharides which normally are difficult to export from the cell, which lead to poor yields, poor broth quality and poor fermentation performance, are produced outside the cell thus eliminating the export problem and thereby potentially increasing product yield and fermentation performance.

Other advantages of this system are control of the by-product formation and the ability to in-situ recycle lactose leading to a very low lactose level at the end of fermentation. In addition, there are several advantages of this process if compared to an in-vitro process where the two oligosaccharides for the transglycosylation reaction are supplied in purified form, such as cost of raw materials, removal of the kinetic equilibrium barrier by recycling of lactose leading to a full conversion of either of the oligosaccharides produced by the cell leading to a higher yield of the desired complex oligosaccharide, possibility to control the by-product composition facilitating purification and removal of lactose at the end of the process.

### SUMMARY

The current application relates to a method for producing one or more oligosaccharides having at least three monosaccharide units, such as i) a balanced mixture of oligosaccharides, ii) a selected oligosaccharide with reduced by-product or iii) an oligosaccharide of at least three or four monosaccharide units, such as a complex sialylated and/or a fucosylated oligosaccharide.

The method is based on the surprising finding that it is possible to culture two strains each producing a different oligosaccharide in the same culture in a controllable manner by controlling the carbon source they are able to grow on.

One aspect is a method for producing one or more oligosaccharides having at least three monosaccharide units, said method comprising the steps of co-culturing a first and a second genetically modified microbial cell in a culture medium, wherein,
a) the first genetically modified microbial cell is capable of producing disaccharide or a first oligosaccharide of at least three monosaccharide units and wherein the genetically modified cell
   i) is capable of growing on a first carbon source while showing limited or no growth on a second carbon source, and
   ii) comprises one or more recombinant nucleic acid sequences encoding at least one glycosyltransferase; and
   iii) comprises at least one pathway to produce an activated sugar nucleotide from the first carbon source; and
b) the second genetically modified microbial cell is capable of producing a second oligosaccharide of at least three monosaccharide units and wherein the genetically modified cell
   i) is capable of growing on the second carbon source while showing limited or no growth on the first carbon source, and
   ii) comprises one or more recombinant nucleic acid sequences encoding at least one glycosyltransferase, and
   iii) comprises a biosynthetic pathway to produce an activated sugar nucleotide from the second carbon source.

In embodiments the ability to grow on one carbon source and not on a second carbon source is achieved by securing the cells express the right transporters for the selected carbon source (sugar transport system) while at the same time not having transporters for the second carbon-source or lacking the capability of utilizing the second carbon sources for growth once it has entered the cell, e.g., kinases needed for phosphorylation of the imported carbon source. If the selective carbon-source growth is not naturally present in the cell it can be genetically engineered to grow and not to grow on the desired carbon sources.

In embodiments the first and second genetically modified microbial cell are capable of independently producing one or more disaccharides or oligosaccharides selected from the group consisting of LNB, LacNAc, 2′FL, 3FL, 2′FLacNAc, 2′FLNB, Lewis A, Lewis X, 3′SL, 6′SL, 3′SLacNAc, 3′SLNB, sialyl-Lewis A, sialyl-Lewis X, DFL, LNT-II, LNT, LNnT, LNFP-I, LNFP-III, LNFP-IV, LNFP-V, LNFP-VI, FSL, LST-a, LST-b, LST-c, LST-d, LNDFH-II and LNDFH-III, DSLNT, pLNH, pLNnH, LNH, LNnH, (D)F-LNH-I, (D)F-LNH-II, (D)F-LNH-III, F-para-LNH-I, DF-para-LNH, DF-para-LNnH, TF-LNH, FLST b, FLST a, FLST-c, S-LNH, S-LNnH-I, FS-LNH, FS-LNnH-I, DS-F-LNH-II.

In further embodiments the first genetically modified cell is capable of producing LacNAc, LNB 2′FL, 3FL LNT-II, LNT, LNnT, LNFP-I, LST-c or LST-a as the most abundant disaccharide or oligosaccharide (e.g., HMO), and preferably the disaccharide or oligosaccharide is transported out of the first genetically modified cell, e.g., by a sugar efflux transporter or a Major Facilitator Superfamily transporter.

In a second aspect the second genetically modified microbial cell is capable of importing the disaccharide or oligosaccharide produced by said first genetically modified microbial cell. In embodiments the import of the disaccharide or oligosaccharide produced by the first genetically modified cell is facilitated by a protein or protein complex selected from table 1 or 2.

In embodiments the second genetically modified cell produces an oligosaccharide with at least three, such as at least four monosaccharide units. Preferably the oligosaccharide of at least three monosaccharide units is selected from Lewis A, Lewis X, sialyl-LacNAc. Preferably the oligosaccharide of at least four monosaccharide units is selected from the group consisting of, sialyl-Lewis X, Lewis B, DFL, FSL, LNT, LNnT, LST-a, LST-b, LST-c, LST-d, LNFP-I, LNFP-II, LNFP-II, LNFP-V, LNFP-VI, LNDFH-I, LNDFH-II, LNDFH-III, DSLNT, pLNH, pLNnH, LNH, LNnH, (D)F-LNH-I, (D)F-LNH-II, (D)F-LNH-III, F-para-LNH-I, DF-para-LNH, DF-para-LNnH, TF-LNH, S-LNFP-I (FLST b), S-LNFP-II (FLST a), S-LNH, S-LNnH-I, FS-LNH, FS-LNnH-I, DS-F-LNH-II, or a mixture of these.

In a third aspect the one or more oligosaccharides produced is a complex oligosaccharide of at least three, such as at least four, monosaccharide units, wherein at least the first or the second genetically modified microbial cell produces a donor oligosaccharide and the other cell produces an acceptor oligosaccharide and said method further comprises the steps of:
a) making an enzyme with transglycosidase activity available in the culture medium and
b) incubating the first disaccharide or the first oligosaccharide, the second oligosaccharide produced in the co-culture with the transglycosidase enzyme in the same culture medium to form a third oligosaccharide of at least four monosaccharide units in the culture medium.

In embodiments the transglycosidase enzyme is selected from the group consisting of α-1,2-tranfucosidase, α-1,3-transfucosidase, α-1,3/4-transfucosidase, α-2,3-transialylase and α-2,6-transialylase and the donor oligosaccharide selected from the group consisting of 2′FL, 3FL, DFL, FSL, LNT, LNnT, sialyl-LacNAc, sialyl-LNB 3′SL and 6′SL and the acceptor oligosaccharide is selected from the group consisting of LacNAc, LNB, 2′FL, 3FL, LNT-II, LNT, LNnT, Para-LNnH, LNFP-I, LNFP-II, LNFP-Ill, LNFP-IV, LNFP-V, LNFP-VI, 3′SL, 6′SL, LST-a, and LST-c.

A further aspect is the use of a composition of oligosaccharides, such as HMOs, produced by the methods described herein in the production of a nutritional composition.

### BRIEF DESCRIPTION OF FIGURES

FIG. 1: Non-limiting illustration of the two-strain system with intermediate oligosaccharide uptake to produce a target HMO with less by-product. A first strain takes up lactose (Lac) via transporter protein 1 (TP-1) the cell is modified to express at least one glycosyltransferase (GT-1+), such as one or two glycosyltransferases, which adds one or more further glycosyl moiety to the lactose (decorates the lactose) to produce a precursor saccharide of at least three monosaccharide units, such as four monosaccharide units, such as and HMO. The precursor saccharide is exported out of strain 1 into the culture medium either by passive diffusion or via a transporter protein 2 (TP-2). The precursor saccharide is internalized by strain 2 via a transporter protein 3 (TP-3). TP-1 and TP-3 may be the same or closely related proteins such as lacY and LacY variants. The second cell is modified to express one or more glycosyltransferases (GT-2+), such as one or two glycosyltransferases, which adds one or more further glycosyl moieties to the precursor saccharide produced by strain 1 to generate the desired oligosaccharide such as an HMO or at least four monosaccharide units, such as five or six or seven monosaccharides. The desired oligosaccharide is preferably exported out of the second stain into the culture medium via a transporter protein 4 (TP-4). The oligosaccharide produced by the 2^{nd} strain may however also be harvested from both the culture medium and the biomass. The first and second strains are engineered such that they do not grow on the same carbon source. Preferably, both strains are inoculated into the bioreactor at the beginning of the fermentation, their growth can be controlled by feeding the two carbon sources at different rates.

FIG. 2: Non-limiting illustration of the two-strain hybrid fermentation-enzymatic process taking place in a fermentation bioreactor. A first oligosaccharide/HMO producing strain in the bioreactor is fed with a first carbon source (e.g., glucose, glycerol, sucrose, fructose, galactose, maltose, sorbitol, arabinose, etc.) and an initial amount of lactose (lac) is provided to produce a first oligosaccharide/HMO. The bioreactor further contains a second oligosaccharide/HMO producing strain which is fed with a second carbon source that is different from the first carbon source (e.g., glucose glycerol, sucrose, fructose, galactose, maltose, sorbitol, arabinose, etc.) and which produces a second oligosaccharide/HMO from the lactose. Preferably, both strains are inoculated into the bioreactor at the beginning of the fermentation, their growth and the production rate of their respective oligosaccharides can be controlled by feeding the two carbon sources at different rates. The first and second oligosaccharide/HMOs produced by the cells can serve as donor and acceptor in the transglycosylation reaction once they are present in sufficient amount in the medium. The transglycosylation is catalyzed by a transglycosidase which is provided to the medium, e.g., by addition or produced by one of the strains. Once the transglycosidase is present in the medium of the running fermentation it catalyzes the transfer a glycosyl moiety from the donor oligosaccharide/HMO (e.g., a sialyl- or fucosyl-lactose), to the acceptor oligosaccharide/HMO thereby generating a complex HMO, such as a third sialylated or fucosylated complex HMO, and lactose as side-product (the leaving group of the enzymatic step). The lactose is in turn taken up by the first and the second strain (recycled) producing more of the first and the second oligosaccharides/HMOs, thereby the equilibrium is pushed towards formation of the third complex HMO. Towards the end of the fermentation feeding of lactose to the fermentation broth can be stopped and the residual lactose consumed until the end of fermentation. The reverse enzymatic reaction is very low if the leaving group (lactose) is recycled faster than the enzymatic reaction rate, which is indicated by the Ø.

FIG. 3: Co-cultivation of a 3′SL strain (MF1) and a LNT strain (MF2) in a 2 L fermenter showing the formation of the desired oligosaccharides 3′SL and LNT, as well as the by-products LNT-II and pLNH2 as the % of the total HMO (mM)+lactose produced over the duration of the fermentation.

FIG. 4: Shows the concentration profile curve in a two-strain hybrid process in weight percentage relative to the total weight of substrates and products (mass fraction %) illustrating the process progress for the synthesis 6′SL (strain MF5) and a LNnT (strain MF6) and the decline of lactose as the two HMOs are produced. 6′SL acts as donor substrate and LNnT is acceptor in the transsialylation reaction that is initiated at 113 h after the start of the fermentation when the α-2,6-transsialidase PITS-197 is added to the fermentation, at this point LST-c starts forming and 6′SL and LNnT levels are reduced while lactose is being formed which stabilizes the 6′SL and LNnT production. Lactose concentration is shown as the dotted line with circles, 6′SL concentration is the shot dashed line with squares, LNnT concentration is the long dashed line with triangles and LST-c concentration is the full line with diamonds.

FIG. 5: Shows the in-vitro enzymatic reaction progress curve in weight percentage relative to the total weight of substrates (mass fraction %) of α-2,6-transsialidase (PITS-197) catalyzed transsialylation of LNnT (acceptor) utilizing 6′SL as a sialyl donor for the synthesis of LST-c. Lactose concentration is shown as the dotted line with circles, 6′SL concentration is the shot dashed line with squares, LNnT concentration is the long dashed line with triangles and LST-c concentration is the full line with diamonds.

FIG. 6: Co-cultivation of a 2′FL strain (MF3) and a 3FL strain (MF4), where the 3FL strain takes up 2′FL as an intermediate substrate for the production of DFL in the MF4 strain.

FIG. 7: Shows the concentration profile curve in a one-strain hybrid process in weight percentage relative to the total weight of substrates and products (mass fraction %) illustrating the process progress addition of 6′SL to an LNnT strain culture and the decline of lactose as the LNnT is produced. 6′SL acts as donor substrate and LNnT is acceptor in the transsialylation reaction initiated with the addition of α-2,6-transsialidase PITS-197 to the fermentation, at this point LST-c starts forming and 6′SL and LNnT levels are reduced. Lactose concentration is shown as the dotted line with circles, 6′SL concentration is the shot dashed line with squares, LNnT concentration is the long dashed line with triangles and LST-c concentration is the full line with diamonds.

FIG. 8 shows the development of lactose, LNT, 3′SL and LST-a in weight % relative to the total weight of the substrates and products in a two-strain process. 3′SL acts as donor substrate and LNT is acceptor in the trans-sialylation reaction initiated with the addition of the α-2,3-transsialidase TcTS to the fermentation, at this point LST-a starts forming and 3′SL and LNT levels are reduced. Lactose concentration is shown as the dotted line with circles, 3′SL concentration is the short dashed line with squares, LNT concentration is the long dashed line with triangles and LST-a concentration is the full line with diamonds.

FIG. 9: Shows the in vitro enzymatic reaction progress curve in weight percentage relative to the total weight of substrates (mass fraction %) of α-2,3-transsialidase TcTS catalyzed trans-sialylation of LNT (acceptor) utilizing 3′SL as a fucosyl donor for the synthesis of LST-a starting at a 1:1 molar ratio of LNT to 3′SL. Lactose concentration is shown as the dotted line with circles, 3′SL concentration is the short dashed line with squares, LNnT concentration is the long dashed line with triangles and LST-a concentration is the full line with diamonds.

FIG. 10: Co-cultivation of a 3′SL strain (MF1) and a LNT strain (MF2) with different ratios of carbon source showing the formation of the desired oligosaccharides 3′SL and LNT, as well as the by-products LNT-II and pLNH2 as the % of the total HMO (mM)+lactose produced over the duration of the fermentation.

FIG. 11: Shows the experimental setup of the regeneration and viability assessment of lyophilized probiotics under pH 3.0 acidic conditions.

FIG. 12: shows colony-forming units (CFU) per milliliter calculated from Lactobacillus rhamnosus (DSM 32550) colonies on agar plates. A) CFU of mix B and C (mixtures with LST-a and LNT) counted on agar plates of dilution step E-2. B) CFU of mix D counted on agar plates of dilution step E-4.

FIG. 13: Shows the regeneration and viability of lyophilized Lactobacillus rhamnosus (DSM 32550), incubated for 3 h at pH 3.0 and plated in two dilutions 1:100 (E-2), 1:1000 (E-3) A) is the control without HMOs B) is Lactobacillus rhamnosus (DSM 32550) in combination with an HMO mixture containing 55% LST-a and 45% LNT (mix B); C) is Lactobacillus rhamnosus (DSM 32550) in combination with an HMO mixture containing 65% LST-a and 55% LNT and 10% 3′SL (mix C); D) is Lactobacillus rhamnosus (DSM 32550) in combination with an HMO mixture containing 25% LNnT and 50% LST-c and 25% 6′SL (mix D).

FIG. 14: Shows the regeneration and viability of lyophilized Bifidobacterium longum (DSM 32946), incubated for 30 min at pH 3.0 and plated undiluted A) is the control without HMOs B) is Bifidobacterium longum (DSM 32946) in combination with an HMO mixture containing 55% LST-a and 45% LNT (mix B); C) is Bifidobacterium longum (DSM 32946) in combination with an HMO mixture containing 65% LST-a and 55% LNT and 10% 3′SL (mix C); D) is Bifidobacterium longum (DSM 32946) in combination with an HMO mixture containing 25% LNnT and 50% LST-c and 25% 6′SL (mix D).

### DETAILED DESCRIPTION

The present invention is based on the surprising finding that it is possible to culture two strains, each producing a different disaccharide or oligosaccharide, in the same culture in a controllable manner by controlling the carbon source they are able to grow on. Essentially, the first genetically modified cell (first strain) producing the first disaccharide or first oligosaccharide is capable of growing on one carbon source while showing limited or no growth on a second carbon source and the second genetically modified cell (second strain) producing the second oligosaccharide is capable of growing on the carbon source which the first cell shows limited or no growth on while it lacks or has limited ability to grow on the carbon source of the first genetically modified cell.

As shown in the examples of the present application this allows for growth of two different stains in the same culture medium, also termed co-culturing, without one strain outgrowing the other strain. The growth on selected carbon sources also allows for simultaneous inoculation of the strains while controlling the product formation from the strains by feeding the carbon sources in selected ratios and or at different time points.

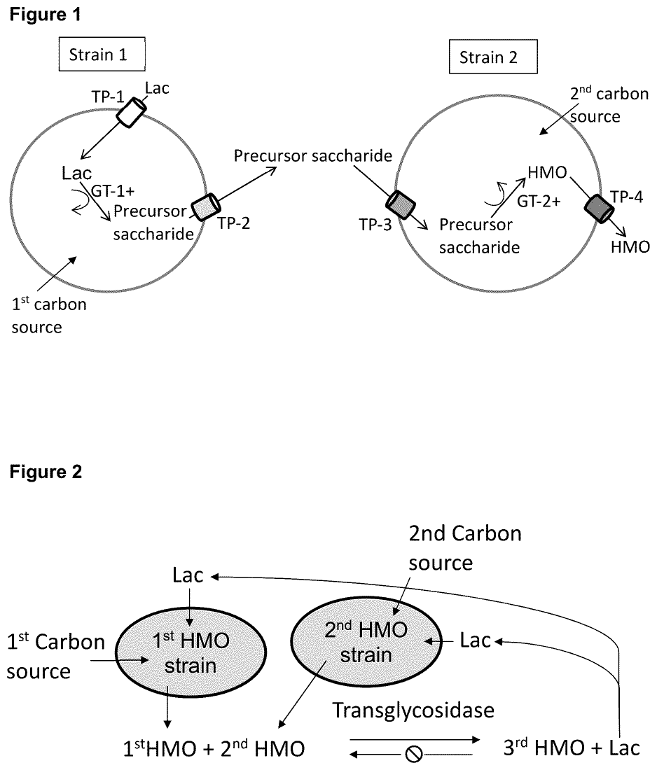

The ability to co-culture two-strains producing two different oligosaccharides can be applied in multiple ways
i) the strains can be used to produce a mixture of the oligosaccharides individually produced by each cell, where the amount of the individual oligosaccharide can be balanced (controlled) by controlling the amount of the different carbon sources. This is the simplest form of the two-strain system, where the two oligosaccharide products are produced independently and there is no interaction or further processing in the culture of the oligosaccharides produced by the cells. One advantage of this system is that it can save production capacity since a mixture of two HMOs can be produced in a single fermentation instead of having to produce the oligosaccharides separately and blend them after production.
ii) the first strain can be used to produce a first oligosaccharide serving as an intermediate oligosaccharide, which is taken up by the second strain which use the internalized intermediate oligosaccharide as substrate to generate a second oligosaccharide (illustrated in FIG. 1). In this application of the two-strain system the second strain is interdependent on the product produced by the first. Hence the production of the second oligosaccharide is dependent of the production of the first oligosaccharide. One advantage of this system is that the formation of oligosaccharide by-products that are usually encountered in single cell systems can be reduced by separating two or more glycosyltransferases in two different cells. The manner that these glycosyltransferases are separated among the two cells depends on the desired HMO outcome/product. Furthermore, reducing the number of glycosyltransferases in a single cell, may also benefit the cell in terms of reducing the metabolic burden imposed on the cell when expressing multiple recombinant/heterologous proteins. Furthermore, separating the glycosyltransferase reactions into two different cells allows for direct control of their respective rates by adjusting the sugar addition profile, which can be used to balance the respective rates to control the by-product formation.
iii) the first strain produces a first oligosaccharide, and the second strain produces second oligosaccharide, where one of the oligosaccharides serves as a donor oligosaccharide and the other oligosaccharide serves as an acceptor oligosaccharide in a transglycosylation reaction occurring in the same culture medium as the two cells are grown in, resulting in the formation of a third complex oligosaccharide (illustrated in FIG. 2). This application of the two-strain system is also termed the hybrid two-strain system since it applies in situ production of a first and a second oligosaccharide (such as HMO) which are reacted in the culture medium with a transglycosidase to produce a third oligosaccharide. One advantage of this system is that complex oligosaccharides which normally are difficult to export from the cell are produced outside the cell elimination the export problem and thereby potentially increasing product yield and fermentation performance. Furthermore, it is possible to have very low amounts of lactose in this system since the lactose leaving the transglycosidase reaction is in-situ recycled by the genetically modified cells to produce additional first and second oligosaccharide for the reaction, leading to a very low lactose level at the end of fermentation and a higher yield of the desired complex oligosaccharide since the kinetic equilibrium barrier towards formation of the third complex oligosaccharide is removed by recycling the leaving group.

In the context of the present invention, the term “donor oligosaccharide” is understood as an oligosaccharide, which provides a specific moiety in a chemical reaction, e.g., a nucleophilic or electrophilic substitution reaction, to a further compound, preferably an acceptor. Likewise, the term “acceptor oligosaccharide” is understood as an oligosaccharide, which receives a specific moiety in a chemical reaction, e.g., nucleophilic or electrophilic substitution reaction, from a donor, thereby forming a third compound.

### Oligosaccharides

In the present context, the term “oligosaccharide” means a sugar polymer containing at least three monosaccharide units, i.e., a tri-, tetra-, penta-, hexa-, hepta-, octa- or higher oligosaccharide. The oligosaccharide can have a linear or branched structure containing monosaccharide units that are linked to each other by interglycosidic linkages. In preferred embodiments, the oligosaccharide comprises a lactose, lacto-N-biose (LNB) or N-acetyllactosamine (LacNAc) residue/moiety at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetylmannosamine, N-acetyl-galactosamine, etc.), uronic acids and ketoaldonic acids (e.g. sialic acid).

In embodiments of the present invention an oligosaccharide to be produced by the method described herein may have a lactose (Galβ1-4Glc) moiety or a lacto-N-biose (LNB or Galβ1-3GlcNAc) moiety or N-acetyllactosamine (LacNAc or Galβ1-4GlcNAc) moiety at the reducing end.

In embodiments the oligosaccharide produced by the method described herein comprises at least 3 monosaccharide units and a lacto-N-biose (LNB or Galβ1-3GlcNAc) moiety or a N-acetyllactosamine (LacNAc or Galβ1-4GlcNAc) moiety at the reducing end, such as for example Lewis A (LeA or Galβ1-3[fucα1-4]GlcNAc) or Lewis X (LeX or Galβ1-4[fucα1-3]GlcNAc), or Lewis Y (LeY or Fucα1-2Gal31-4[Fucα1-3]GlcNAc) or Lewis-B (LeB or Fucα 1-2Galβ1-3[Fucα1-4]GlcNAc) or 3′sialyl-LNB (Neu5Ac-α2-3Galβ1-3-GlcNAc) 3′sialyl-lacNAc (Neu5Ac-α2-3Galβ1-4-GlcNAc) or 6′sialyl-lacNAc (6′LN or Neu5Ac-α2-6Galβ1-4-GlcNAc) sialyl-Lewis A (SLeA or Neu5Ac-α2-3Galβ1-3[fucα1-4]GlcNAc) or silayl-Lewis X (SLeX or Neu5Ac-α2-3Galβ1-4[fucα1-3]GlcNAc).

In the context of the present invention a complex oligosaccharides is an oligosaccharide that fall into one of the following three categories i) oligosaccharides composed of at least four monosaccharide units of which at least two are selected from a fucosyl and/or a sialyl moiety, ii) oligosaccharides composed of at least five monosaccharide units, preferably with at least on sialyl or fucosyl monosaccharide, and iii) oligosaccharides composed of at least six monosaccharide units, preferably neutral oligosaccharides. In the context of the present disclosure, a sub-category of the complex oligosaccharides are the highly complex oligosaccharides where at least one monosaccharide unit in the oligosaccharide comprises at least three glycosidic linkages to additional monosaccharide units.

### HMOs

Preferred oligosaccharides of the disclosure are human milk oligosaccharides (HMOs).

The term “human milk oligosaccharide” or “HMO” in the present context means a carbohydrate found in human breast milk. The HMOs have a core structure comprising a lactose unit at the reducing end that can be elongated by one or more beta-N-acetyl-lactosaminyl and/or one or more beta-lacto-N-biosyl units, and this core structure can be substituted by an alpha-L-fucopyranosyl (fucosylated) and/or an alpha-N-acetyl-neuraminyl moiety (sialylated). HMO structures are for example disclosed in by Xi Chen in Chapter 4 of Advances in Carbohydrate Chemistry and Biochemistry 2015 vol 72.

In the context of the present disclosure, lactose is not regarded as an HMO species, but a substrate for the process. It is preferred to reduce lactose as much as possible at the end of the process.

HMOs are either neutral or acidic. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated.

Examples of such neutral non-fucosylated HMOs (neutral core HMOs) include lacto-N-triose II (LNT-II), lacto-N-tetraose (LNT or Galβ1-3GlcNAcβ1-3Galβ1-4Glc), lacto-N-neotetraose (LNnT or Galβ1-4GlcNAcβ1-3Galβ1-4Glc), lacto-N-neohexaose (LNnH or Galβ1-4GlcNAcβ1-3(Galβ1-4GlcNAcβ1-6)Galβ1-4Glc), para-lacto-N-neohexaose (pLNnH or Galβ1-4GlcNAcβ1-3Galβ1-4GlcNAcβ1-3Galβ1-4Glc), para-lacto-N-hexaose (pLNH or Galβ1-3GlcNAcβ1-3Galβ1-4GlcNAcβ1-3Galβ1-4Glc) and lacto-N-hexaose (LNH or Galβ1-3GlcNAcβ1-3(Galβ1-4GlcNAcβ1-6)Galβ1-4Glc).

Examples of neutral fucosylated HMOs include 2′-fucosyllactose (2′FL or Fucα1-2Galβ1-4Glc), 3-fucosyllactose (3FL or Galβ1-4(Fucα1-3)Glc), difucosyllactose (DFL or LDFT or Fucα1-2Gal1-4(Fucα1-3)Glc), lacto-N-fucopentaose I (LNFP-I or Fucα 1-2Galβ1-3GlcNAcβ1-3Galβ1-4Glc), lacto-N-fucopentaose II (LNFP-II or Galβ1-3[Fuc-α1-4]GlcNAcβ1-3Galβ1-4Glc), lacto-N-fucopentaose III (LNFP-III or Galβ(1-4[Fuc-α1-3]GlcNAcβ1-3Galβ1-4Glc), lacto-N-fucopentaose IV (LNFP-IV or Fuc-α1-2Galβ1-4GlcNAcβ1-3Galβ1-4Glc), lacto-N-fucopentaose V (LNFP-V or Galβ1-3GlcNAcβ1-3Galβ1-4[Fuc-α1-3]Glc), lacto-N-fucopentaose VI (LNFP-VI or Galβ1-4GlcNAcβ1-3Galβ1-4[Fucα1-3]Glc), lacto-N-difucohexaose I (LNDFH-I or Fucα1-2Galβ1-3[Fucα1-4]GlcNAcβ1-3Galβ1-4Glc), lacto-N-difucohexaose II (LNDFH-II or Galβ1-3[Fuc-(α1-4)]GlcNAcβ1-3Galβ1-4[Fucα1-3]Glc), lacto-N-difucohexaose III (LNDFH-II or Galβ(1-4[Fuc-(α1-3)]GlcNAcβ1-3Galβ1-4[Fucα1-3]Glc), fucosyl-lacto-N-hexaose I (FLNH-I or Fuc-α1-2Galβ1-3GlcNAcβ1-3(Galβ1-4GlcNAcβ1-6)Galβ1-4Glc), fucosyl-lacto-N-hexaose II (FLNH-II or Galβ1-3(Fuc-α1-4)GlcNAcβ1-3(Galβ1-4GlcNAcβ1-6)Galβ1-4Glc), fucosyl-lacto-N-hexaose III (FLNH-III or Galβ1-3GlcNAcβ1-3 ((Gal(β1-4(Fucα1-3)GlcNAcβ1-6))Galβ1-4Glc), fucosyl-para-lacto-N-hexaose I (FpLNH-I or Galβ1-3GlcNAcβ1-3Galβ1-4[Fuc-(α1-3)]GlcNAcβ1-3Galβ1-4Glc), fucosyl-para-lacto-N-neohexaose II (FpLNnH-II or Galβ1-4GlcNAcβ1-3Gal(fucα1-3)β1-4GlcNAcβ1-3Galβ1-4Glc), Difucosyl-Lacto-N-hexaose I (DF-LNH-I or DF-LNHa or fuc-α1-2Galβ1-3GlcNAcβ1-3 ((Gal(β1-4(Fuc1-3)GlcNAcβ1-6))Galβ1-4Glc), Difucosyl-Lacto-N-hexaose II (DF-LNH-II or DF-LNHb or Galβ1-3(fuc-α1-4)GlcNAcβ1-3 ((Gal(β1-4(Fuc1-3)GlcNAcβ1-6))Galβ1-4Glc), Difucosyl-Lacto-N-hexaose III (DF-LNH-III or DF-LNHc or fuc-α1-2Galβ1-3(fuc-α1-4)GlcNAcβ1-3 (Gal(β1-4GlcNAcβ1-6)Galβ1-4Glc), Difucosyl-para-lacto-N-hexaose (DF-para-LNH or Galβ1-3(fuc-α1-4)GlcNAcβ1-3Galβ1-4(fuc-α1-3)GlcNAcβ1-3Galβ1-4Glc), Difucosyl-para-lacto-N-neohexaose (DF-para LNnH or Galβ1-4[Fucα1-3]GlcNAcβ1-3Galβ1-4[Fuc-α1-3]-GlcNAcβ1-3Galβ1-4Glc), fucosyl-lacto-N-neohexaose a (FLNnHa), fucosyl-lacto-N-neohexaose b (FLNnHb), difucosyl-lacto-N-neohexaose (DFLNnH) and trifucosyl-lacto-N-hexaose (TF-LNH or fuc-α1-2Galβ1-3(fuc-α1-4)GlcNAcβ1-3 ((Gal(β1-4(Fuc1-3)GlcNAcβ1-6))Galβ1-4Glc).

Examples of acidic HMOs include 3′-sialyllactose (3′SL or Neu5Ac-α2-3Galβ1-4-Glc), 6′-sialyllactose (6′SL or Neu5Ac-α2-6Galβ1-4-Glc), 3-fucosyl-3′-sialyllactose (FSL or Neu5Ac-α2-3Gal1-4(Fucα1-3)Glc), 3′-sialyllacto-N-tetraose a (LST a or Neu5Ac-α2-3Galβ1-3GlcNAcβ1-3Galβ1-4Glc), fucosyl-LST a (FLST a or Neu5Ac-α2-3Gal1-3(Fucα1-4)GlcNAcβ1-3Galβ1-4Glc), 6′-sialyllacto-N-tetraose b (LST b or Galβ1-3(Neu5Ac-α2-6)GlcNAcβ1-3Galβ1-4Glc), fucosyl-LST b (FLST b or Fucα1-2Galβ1-3(Neu5Ac-α2-6)GlcNAcβ1-3Galβ1-4Glc), 6′-sialyllacto-N-neotetraose (LST c or Neu5Ac-α2-6Galβ1-4GlcNAcβ1-3Galβ1-4Glc), fucosyl-LST c (FLST c or Neu5Ac-α2-6Galβ1-4GlcNAcβ1-3Gal1-4(Fucα1-3)Glc), 3′-sialyllacto-N-neotetraose (LST d), fucosyl-LST d (FLST d), disialyl-lacto-N-tetraose (DSLNT or Neu5Ac-α2-3Galβ1-3(Neu5Ac-α2-6)GlcNAcβ1-3Galβ1-4Glc), Sialyl-para-lacto-N-neohexaose (S-pLNnH or Neu5Ac-α2-3Galβ1-4GlcNAcβ1-3Galβ1-4GlcNAcβ1-3Galβ1-4Glc), sialyl-lacto-N-hexaose (SLNH or Neu5Ac-α2-6Galβ1-4GlcNAcβ1-6(Galβ1-3GlcNAcβ1-3)Galβ1-4Glc), fucosyl-sialyl-lacto-N-hexaose (FSLNH or Neu5Ac-α2-6Galβ1-4GlcNAcβ1-6(Fucα1-2Galβ1-3GlcNAcβ1-3)Galβ1-4Glc), sialyl-lacto-N-neohexaose I (SLNnH-I or Neu5Ac-α2-3Galβ1-4GlcNAcβ1-6(Galβ1-4GlcNAcβ1-3Galβ1-4Glc), fucosyl-sialyl-lacto-N-neohexaose I (FSLNnH-I or Neu5Ac-α2-6Galβ1-4GlcNAcβ1-3(Gal1-4(Fucα1-3)GlcNAcβ1-6)Galβ1-4Glc), sialyl-lacto-N-neohexaose II (SLNnH-II or Neu5Ac-α2-6Galβ1-4GlcNAcβ1-3(Galβ1-4GlcNAcβ1-6)Galβ1-4Glc) and Disialyl-fucosyl-lacto-N-hexaose II (DS-FLNH-II or Neu5Ac-α2-3Galβ1-3(Neu5Ac-α2-6)GlcNAcβ1-3(Galβ1-4(Fucα1-3)GlcNAcβ1-6)Galβ1-4Glc).

In the context of the present disclosure a complex HMO is an HMO that fall into one or more of the following three categories i) HMOs composed of at least four monosaccharide units of which at least two are selected from a fucosyl and/or a sialyl moiety, ii) HMOs composed of at least five monosaccharide units, preferably with at least on sialyl or fucosyl monosaccharide, non-limiting examples being LNFP-I, LNFP-II, LNFP-V, LST-a, LST-c as well as many of the highly complex HMOs mentioned below, and iii) HMOs composed of at least six monosaccharide units, preferably neutral non-fucosylated oligosaccharides such as pLNH-I, pLNnH LNH and LNnH. In the context of the present disclosure, a sub-category of the complex HMOs are the highly complex HMOs where at least one monosaccharide unit in the HMO comprises at least three glycosidic linkages to additional monosaccharide units. Non-limiting examples of highly complex HMOs are LNH, LNnH, LNFP-II, LNFP-III, LST-b, DSLNT, LNDFH-I, LNDFH-II, FLST-a, FLST-b, FpLNnH, FpLNnHII, F-LNH-I, F-LNH-II, DF-LNH-I, DF-LNH-II, DF-LNH-III, TF-LNH, DFpLNH, DFpLNnH, S-LNFP-I, S-LNH, S-LNnH-I, FS-LNH, FS-LNnH-I and DS-F-LNH-II.

Preferably, the complex HMOs of the present invention are not readily exported from the cytosol of the cell to the supernatant if produced by fermentation. Complex HMOs produced by the two-strain hybrid method described herein requires the action of at least three enzymes. The three enzymes can for example be at least two glycosyltransferases present in the cytosol of each of the genetically engineered cells used in the process and a transglycosidase present in the culture medium of the fermentation. For example, FSL produced using the two-strain hybrid system described herein requires the presence of an alpha-1,3-fucosyltransferase enzyme inside one of the genetically modified cells to form 3FL and an alpha-2,3-sialyltransferase inside the second cell to form 3′SL, where both 3FL and 3′SL is exported to the culture medium, and an alpha-2,3-transsialidase enzyme in the culture broth to from FSL from the 3FL and 3′SL produced by the cells. Export from the genetically modified cell into the culture medium may require the presence of a recombinant transport in the genetically modified cell. For non-limiting examples of suitable transporters see for example WO2010/142305, WO2021/148615, WO2021/148614, WO2021/148611, WO 2021/148610, WO2021/148620 and WO2021/148618.

In one method according to the present description, the fucosylated and/or sialylated oligosaccharide, such as a human milk oligosaccharide (HMO), having at least four monosaccharide units is an HMO of four monosaccharide units, such as DFL or FSL. Preferably, the complex HMO of four monosaccharide units is FSL.

In one method according to the present description, the fucosylated and/or sialylated oligosaccharide, such as a human milk oligosaccharide (HMO), having at least four monosaccharide units, is an oligosaccharide with five monosaccharide units, such as an oligosaccharide selected from the group consisting of LNFP-I, LNFP-II, LNFP-III, LNFP-IV, LNFP-V, LNFP-VI, LST-a, LST-b, LST-c and LST-d. Specifically, the fucosylated and/or sialylated HMO with five monosaccharide units may be selected from an HMO the group consisting of LNFP-I, LNFP-II, LNFP-III, LNFP-V, LNFP-VI, LST-a, LST-b and LST-c.

In one method according to the present description, the fucosylated and/or sialylated oligosaccharide, such as a human milk oligosaccharide (HMO), having at least four monosaccharide units is an HMO with six monosaccharide units. Specifically, the fucosylated and/or sialylated HMO with six monosaccharide units may be selected from the group consisting of DSLNT, LNDFH-I, LNDFH-II, LNDFH-III, FLST-a, FLST-b and FLST-c.

In one aspect, the method according to the present description produces a human milk oligosaccharide (HMO) of seven or eight monosaccharide units, such as an HMO selected from the group consisting FLNH-I, FLNH-II, FLNH-III, FpLNH-I, FpLNnH II, DF-LNF-I, DF-LNF-II, DF-LNF-III, DF-para-LNH, DF-para LNnH, FLNnHa, FLNnHb, DFLNnH, TF-LNH, SLNH, FSLNH, SLNnH-I, FSLNnH-I, SLNnH-II, and DS-FLNH-II. Production of these HMOs may require the presence of three or more glycosyltransferase and/or transglycosidase activities.

### Two-Strain System

The two-strain oligosaccharide production system disclosed herein is based on the ability to grow two different genetically modified cells in the same culture medium using their ability to grow on non-identical carbon sources. Hence, the cells are cultured in the same vessel, where the vessel does not contain any means of separating the components in the vessel.

One aspect of the present disclosure is a method for producing one or more oligosaccharides having at least three monosaccharide units, said method comprising the steps of co-culturing a first and a second genetically modified microbial cell in a culture medium, wherein,
a) the first genetically modified microbial cell is capable of producing a first disaccharide or a first oligosaccharide of at least three monosaccharide units and wherein the genetically modified cell
   i) is capable of growing on a first carbon source while showing limited or no growth on a second carbon source, and
   ii) comprises one or more recombinant nucleic acid sequences encoding at least one glycosyltransferase; and
   iii) at least one pathway to produce a nucleotide-activated sugar from the first carbon source; and
   iv) is preferably capable of exporting said first oligosaccharide into the culture medium, and
b) the second genetically modified microbial cell is capable of producing a second oligosaccharide of at least three monosaccharide units and wherein the genetically modified cell
   i) is capable of growing on the second carbon source while showing limited or no growth on the first carbon source, and
   ii) comprises one or more recombinant nucleic acid sequences encoding at least one glycosyltransferase, and
   iii) comprises at least one biosynthetic pathway for making said activated sugar nucleotide from the second carbon source; and
   iv) is preferably capable of exporting said second oligosaccharide into the culture medium.

The most common carbon sources used in bioproduction of oligosaccharides are glucose, glycerol and sucrose, and these are the preferred carbons source since these are the economically most attractive. Other alternative carbon sources are for example fructose, galactose, sorbitol, arabinose and maltose.

In embodiment of the present description the first and second carbon sources are different, and they are preferably selected from the group consisting of glucose, glycerol, sucrose, fructose, galactose, sorbitol, arabinose and maltose.

To make the use of fructose, galactose and maltose economically relevant these may be obtained from cheaper sources.

Fructose could possible also be obtained from hydrolyzed sucrose, or high fructose-sirup (made from glucose). The use of hydrolyzed sucrose could for example be an advantage in the event that one cell grow on fructose and not on glucose and the other cell grow on glucose and not on fructose, in this case both the first and the second genetically modified cell could be fed using hydrolyzed sucrose resulting in a 50:50 mixture of fructose and glucose, a ratio which can be changed by adding either high fructose sirup or glucose to the culture medium, e.g. through the feed.

Hydrolyzed lactose is another option to obtain glucose and galactose. Hence in the event that one cell grows on galactose and not on glucose and the other cell grows on glucose and not on galactose the first and the second genetically modified cell could be fed using hydrolyzed lactose resulting in a 50:50 mixture of galactose and glucose, a ratio which can be changed by adding either galactose or glucose to the culture medium, e.g. through the feed.

Glucose syrup made from starch rich sources such as, but not limited to corn, potatoes, rice, wheat, and barley, contain glucose as well as maltose and longer non-fermentable sugars such as maltotriose. High maltose syrup can also be made from starch sources using a maltogen amylase that mostly produce maltose and not glucose. Glucose syrup is also known as a carbon and energy source in the formation of bioethanol, but may also be used in a bioproduction described herein where one cell grow on maltose and not on glucose and the other cell grow on glucose and not on maltose.

The inventors of the present application have realized that the growth of a first and a second genetically modified strain in the same culture medium can be controlled if the strains have different abilities to grow on two selected carbon sources. Preferably, the first genetically modified cell grows on a first carbon source while it shows limited or no growth on a second carbon source. The second genetically modified cell on the other hand grow on the second carbon source while it shows limited or no growth on the first carbon source.

The terms “culturing” or “fermenting” or “fermentation” are used interchangeably in the present description and refers to the growth of the genetically modified cells (strains) in a bioreactor with the purpose of producing the first and the second oligosaccharide. A culture encompasses both cells and liquid and is also known as the culture broth. The culture medium is the liquid in which the cells are capable of growing. Products in the culture broth or culture medium or enzymatic reactions occurring in the culture broth or culture medium are to be understood as being/occurring outside the cells in the culture medium.

The term “co-culture” or “co-culturing” as used in the present disclosure relates to growth of two different genetically modified cells (strains) in the same culturing vessel, such as a shake flask, a fermenter or a bioreactor, to produce their products into the same culture medium. In the present disclosure it is understood that the two different strains are not separated by a semipermeable membrane. Preferably, the two different strains are grown simultaneously in the culture medium allowing free distribution of cells and ingredients and metabolites and product. The two different strains may for example be inoculated into the vessel at the beginning of the cultivation. This allows the strains to grow simultaneously in the same vessel producing their products in the same culture medium. The second strain may however also be added to the vessel at a later timepoint if it is desired to give the first strain an opportunity to increase its biomass before the second strain is added. This will still result in simultaneous growth of the strains during some of the cultivation time. The two different strains may also be inoculated (pitched) in different ratios (cells/ml), if it is known that one strain has an initial slower growth than the other strain, or if the oligosaccharide yield per mol of carbon source of the two strains are different, or if it is desired that the ratio of the products produced by the two strains is different. The term cell and strain are used interchangeably in the present disclosure. In the co-culture the strains do not necessarily need to produce their products simultaneously, since the production of one product may be dependent of the production of one or more intermediate products.

A natural microbial fermentation follows four phases, namely the lag phase, the growth phase, the stationary phase and the death phase. In industrial fed-batch fermentations the cells grow in two phases, a first phase of rapid cell growth in a culture medium with either unrestricted access to a carbon source or restricted access following a rapidly increasing feeding profile which limits the carbon source, and a second phase of more controlled cell growth where the industrial product is often produced. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter. In the two-strain method described herein, the cultivation is preferably a fed-batch or a continuous fermentation using two different carbon sources.

Preferably, the cultivation is started with at least one initial carbon source (batch phase), and when this is consumed the carbon source is fed at a desired rate to the culture medium throughout the fermentation (feeding phase). When operating with two different strains feeding on two different carbon sources the fermentation may essentially contain two batch phases running simultaneously, one for each strain/carbon source. The batch phase (biomass formation) may be conducted on a different carbon source than the carbon source used for production of the oligosaccharide in the feeding phase (carbon-limiting conditions). This may in particular be useful if one strain has a slower growth rate than the other strain on the “production” carbon source. The batch phase may also be conducted with the same carbon source for the two strains, which is then a third carbon source, since the two different carbon sources used in the feeding phase (production) does not allow one of the two strains to grow effectively. The feeding rates of the two carbon sources in the production phase can be predicted based on knowledge of the individual growth rates and oligosaccharide product yields of the first and second strains. If a specific ratio of oligosaccharides is desired, this can be achieved by balancing the ratio of the two carbon sources in the feed based on the oligosaccharide/carbon source yield of the individual strains. As indicated above the batch phases may also be staggered, if it is desired to start the growth of one strain ahead of the other strain. The industrial fermentation will always be stopped before significant cell death occurs.

In addition to the carbon source used for growth of the cells, the cells will also need a substrate for the formation of the oligosaccharides, the substrate is generally different from the growth carbon source. Most commonly the substrate is lactose, which can either be added to the culture or produced by the cells themselves. Alternative substrates may also be used to produce the first and/or second oligosaccharides. Alternative substrates are described in the two-strain hybrid system below. If one of the modified strains produce a disaccharide there may not be a need for an additional substrate in that the cell can make the disaccharide from the growth carbon source.

In order for the first and second genetically modified cells to grow on different carbon sources and not on the same carbon source it may be necessary to select cells with certain growth properties or genetically modify the cells such that they have the desired growth properties.

Microorganisms are often capable of using more than one carbon source to facilitate its growth. To establish whether a cell can grow on a certain carbon source one may for example spread the microorganism on an agar plate with the selected carbon source and observe the formation of colonies.

Depending on the species of the microbial strain used in the context of the present description different modifications may be needed to secure that a strain does not grow or has limited growth on the desired carbon source.

In the context of the co-culturing described herein, limited or reduced growth of a genetically modified cell refers to a cell that has a reduced affinity and uptake rate for a specific carbon source (low affinity strain) which means it cannot effectively compete with a strain having a higher affinity for the same specific carbons source. This is especially the case when the growth on the specific carbon source is under carbon limited conditions such as in the feeding phase of a carbon limited fed-batch or a continuous culture, in such a case the higher affinity strain will lower the residual concentration of the specific carbon source in the medium to such low levels that the low affinity strain has almost no growth. Moreover, even in a batch phase with excess carbon source the low affinity strain is at a major disadvantage compared to the high affinity strain as the maximum growth rate is also affected by lack of a main carbon source uptake system for the specific carbon source (e.g., deletion of the ptsG when the specific carbon source is glucose) since the maximum carbon source uptake rate, and thereby also maximum growth rate, is affected.

Gram negative cells are known to have a periplasmic space between the inner cytoplasmic membrane and the bacterial outer membrane. Gram positive bacteria can also have a periplasmic space although this is often significantly smaller. In terms of prevention or limiting growth on a specific carbon source in a microbial cell an option is to prevent the carbon source to enter the cytosol of the microbial cell, hence a carbon source may enter the periplasmic space, but if it is prevented to enter the cytosol the cell may still not be able to grow on it. A further option is to prevent the cell to further process the carbon source once it enters the cytosol, such that it cannot enter energy producing pathways such as glycolysis, pentose phosphate pathway or the Krebs cycle which the cell needs to grow. This can be achieved by denying the cell access to the enzyme needed to phosphorylate the carbon source. This entails abolishing its uptake via the PTS-uptake system if such as system is present for the carbon source and/or removing the enzyme responsible for its phosphorylation inside the cytosol.

In embodiments the ability to grow on a first carbon source and not on a second carbon source is achieved by securing that the cells express the right transporters for the selected carbon source while at the same time not having efficient transporters for the second carbon-source. If the desired transporters are or are not naturally present in the host cell, the cell can be genetically engineered to exhibit the desired carbon-source utilization.

In the sections below transport and utilization of the different carbon sources, glucose, glycerol, sucrose, galactose, fructose, sorbitol, arabinose and maltose is described. If alternative carbon sources are desired the skilled person will know how to modify the cells to accommodate these as well. It is understood that in the method described herein the genetically modified cells may have functional transport/utilization of a sugar selected from glucose, glycerol, sucrose, galactose, fructose, sorbitol, arabinose and maltose, or one or more of the transporter or utilization enzymes of a sugar selected from glucose, glycerol, sucrose, galactose, fructose, sorbitol, arabinose and maltose may be reduced or abolished, e.g., by mutation or deletion of relevant genes described in the sections below.

In some embodiments the first genetically modified microbial cell grows on sucrose and has no or limited growth on glucose and the second genetically modified microbial cell grows on glucose and has no or limited growth on sucrose.

In some embodiments the first genetically modified microbial cell grows on sucrose and has no or limited growth on glycerol and the second genetically modified microbial cell grows glycerol and has no or limited growth on sucrose.

In some embodiments the first genetically modified microbial cell grows on sucrose and has no or limited growth on maltose and preferably also on glucose and the second genetically modified microbial cell grows maltose and has no or limited growth on sucrose.

In some embodiments the first genetically modified microbial cell grows on sucrose and has no or limited growth on galactose and the second genetically modified microbial cell grows galactose and has no or limited growth on sucrose.

In some embodiments the first genetically modified microbial cell grows on sucrose and has no or limited growth on fructose and the second genetically modified microbial cell grows fructose and has no or limited growth on sucrose.

In some embodiments the first genetically modified microbial cell grows on sucrose and has no or limited growth on arabinose and the second genetically modified microbial cell grows on arabinose and has no or limited growth on sucrose.

In some embodiments the first genetically modified microbial cell grows on sucrose and has no or limited growth on sorbitol and the second genetically modified microbial cell grows on sorbitol and has no or limited growth on sucrose.

In some embodiments the first genetically modified microbial cell grows on glucose and has no or limited growth on sucrose and the second genetically modified microbial cell grows on sucrose and has no or limited growth on glucose.

In some embodiments the first genetically modified microbial cell grows on glucose and has no or limited growth on glycerol and the second genetically modified microbial cell grows on glycerol and has no or limited growth on glucose.

In some embodiments the first genetically modified microbial cell grows on glucose and has no or limited growth on galactose and the second genetically modified microbial cell grows on galactose and has no or limited growth on glucose.

In some embodiments the first genetically modified microbial cell grows on glucose and has no or limited growth on fructose and the second genetically modified microbial cell grows on fructose and has no or limited growth on glucose.

In some embodiments the first genetically modified microbial cell grows on glucose and has no or limited growth on arabinose and the second genetically modified microbial cell grows on arabinose and has no or limited growth on glucose.

In some embodiments the first genetically modified microbial cell grows on glucose and has no or limited growth on sorbitol and the second genetically modified microbial cell grows on sorbitol and has no or limited growth on glucose.

In some embodiments the first genetically modified microbial cell grows on glycerol and has no or limited growth on sucrose and the second genetically modified microbial cell grows on sucrose and has no or limited growth on glycerol.

In some embodiments the first genetically modified microbial cell grows on glycerol and has no or limited growth on glucose and the second genetically modified microbial cell grows on glucose and has no or limited growth on glycerol.

In some embodiments the first genetically modified microbial cell grows on glycerol and has no or limited growth on galactose and the second genetically modified microbial cell grows on galactose and not or limited on glycerol.

In some embodiments the first genetically modified microbial cell grows on glycerol and has no or limited growth on fructose and the second genetically modified microbial cell grows on fructose and not or limited on glycerol.

In some embodiments the first genetically modified microbial cell grows on glycerol and has no or limited growth on maltose and preferably also on glucose and the second genetically modified microbial cell grows on maltose and not or limited on glycerol.

In some embodiments the first genetically modified microbial cell grows on glycerol and has no or limited growth on arabinose and the second genetically modified microbial cell grows on arabinose and has no or limited growth on glycerol.

In some embodiments the first genetically modified microbial cell grows on glycerol and has no or limited growth on sorbitol and the second genetically modified microbial cell grows on sorbitol and has no or limited growth on glycerol.

In some embodiments the first genetically modified microbial cell grows on galactose and has no or limited growth on maltose and preferably also on glucose and the second genetically modified microbial cell grows on maltose and not or limited on galactose.

In some embodiments the first genetically modified microbial cell grows on galactose and has no or limited growth on fructose and preferably also on glucose and the second genetically modified microbial cell grows on maltose and not or limited on galactose.

In some embodiments the first genetically modified microbial cell grows on fructose and has no or limited growth on maltose and preferably also on glucose and the second genetically modified microbial cell grows on maltose and not or limited on fructose.

In embodiments the one or more oligosaccharides produced by the co-culture is a mixture of at least two human milk oligosaccharides (HMOs). Preferably, the at least two oligosaccharides are harvested from the co-culture. The HMOs produced by the first and second genetically modified microbial cell may independently be selected from the group consisting of 2′FL, 3FL, 3′SL, 6′SL, DFL, LNT-II, LNT, LNnT, LNFP-I, LNFP-III, LNFP-V, LNFP-VI, FSL, LST-a, LST-b, LST-c, LST-d, LNDFH-II and LNDFH-III, DSLNT, pLNH, pLNnH, LNH, LNnH, (D)F-LNH-I, (D)F-LNH-II, (D)F-LNH-III, F-para-LNH-I, DF-para-LNH, DF-para-LNnH, TF-LNH, FLST b, FLST a, FLST-c, S-LNH, S-LNnH-I, FS-LNH, FS-LNnH-I, DS-F-LNH-II.

In preferred embodiments the HMOs produced by the first and second genetically modified microbial cell may independently be selected from the group consisting of 2′FL, 3FL, 3′SL, 6′SL, DFL, LNT-II, LNT, LNnT, LNFP-I, LNFP-II, LNFP-II, LNFP-V, LNFP-VI, LST-a, LST-c, LNDFH-I, LNDFH-II and LNDFH-III.

### Glucose Transport and Utilization

Glucose is one of the most accepted carbon sources by microorganisms and there are multiple systems by which a microbial cell can take up glucose and convert it into energy for growth. Various glucose transport systems are well described, see for example Jaheris et al 2008 FEMS Microbiol Rev 32: 891-907 for bacteria, Fuentes et al. 2013 Microbial Cell Factories 12:42 for E. coli and Kim et al 2013 Biochimica et Biophysica Acta 1830: 5204-5210 for yeast.

In embodiments described herein, a cell that grow on glucose has at least one glucose transport system. The glucose transport system may be selected from the systems described in Jaheris et al, Fuents et al or Kim et al. Specifically the glucose import system may be selected from a phosphoenolpyruvate:sugar phosphotransferase systems (PTS) such as PTS-dependent glucose (glc) utilization system, PTS-dependent mannose (man) utilization system, PTS-dependent maltose (mal) utilization system, PTS-dependent beta-glucoside (bgl) utilization system or PTS-dependent N-acetylglucosamine (nag) utilization system. Alternative glucose transport systems are the galactose:H+ symporter GalP, glucose uptake protein GlcU, sodium/glucose transporter family (SGLT) or ABC transporters such as the galactose/glucose ABC transporter (mglABC) system, trehalose/maltose/sucrose/palatinose (TMSP)-ABC transporter (malEFG) system and glucose/mannose ABC transporter (glcEFG) system or MFS transporter systems such as glucose proton symporter (glcP) and glucose facilitator (glf) or hexose transporters (HXT).

In other embodiments reduction or prevention of import of glucose into the cytosol of a microorganism can be achieved by mutating or deleting one or more sequences encoding proteins that affect the glucose import capacity as described in the paragraph above.

To utilize the glucose to make energy the cell phosphorylates the glucose once it has entered the cytosol to glucose-6-phosphate which can enter the energy producing metabolic pathways such as glycolysis and pentose phosphate pathway. Blocking the formation of glucose-6-phosphate may therefore also serve to prevent the cell to utilize glucose as a carbon source for growth.

In embodiments the genetically modified microbial cell is a bacterium that has reduced or no growth on glucose, wherein the functionality of one or more endogenous proteins involved in glucose import and utilization, in said cell can be reduced or abolished. Preferably the proteins are selected from the group consisting of
i) glucose PTS complex components IICB^{Glc} (ptsG, e.g., Uniprot accession nr P69786, or functional variants thereof);
ii) beta-glucoside PTS complex components IIABC^{Bgl} (bglF, e.g., Uniprot accession nr P08722, or functional variants thereof);
iii) mannose PTS complex components—IICD^{Man} (manX, e.g., Uniprot accession nr P69797, or functional variants thereof);
iv) N-acetylglucosamine PTS complex components IIABC^{Nag} (nagE, e.g., Uniprot accession nr P09323, or functional variants thereof);
v) maltose/maltodextrin transport system (malX, e.g., Uniprot accession nr P19642, or functional variants thereof);
vi) galactose/glucose high-affinity ABC transporter components (mglC, e.g., Uniprot accession nr P23200, or functional variants thereof);
vii) trehalose/maltose/sucrose/palatinose (TMSP)-ABC transporter (malF, e.g., Uniprot accession nr P02916 or functional variants thereof);
viii) trehalose/maltose/sucrose/palatinose (TMSP)-ABC transporter (malG, e.g., Uniprot accession nr P68183, or functional variants thereof);
ix) galactose permease (galP, e.g., Uniprot accession nr P0AEP1, or functional variants thereof);
x) glucose proton symporter (glcP, e.g., Uniprot accession nr O07563, or functional variants thereof);
xi) glucose facilitator (glf, e.g., Uniprot accession nr P37747 or P21906, or functional variants thereof);
xii) glucose uptake protein (glcU, e.g., Uniprot accession nr P40420, or functional variants thereof);
xiii) sodium/glucose transporter family (sglT, e.g., Uniprot accession nr P96169, or functional variants thereof); and
xiv) glucokinase (glk, e.g., Uniprot accession nr P0A6V8, or functional variants thereof); and
xv) hexose transporters (HXT).

Where the proteins in i)-vi) and xv) are all part of various glucose import complexes which generally are composed of multiple proteins. The proteins in item vii)-xiii) are single protein transports identified in different bacterial species. The protein in xiv) is an example of a glucose utilization enzyme, which phosphorylates glucose once it has entered the cell. In context of the present disclosure, it is preferred to reduce or abolish the activity of the membrane bound transporter protein. Preferably, the gene to be mutated/deleted in the mentioned complex is indicated in brackets in italics.

In embodiments the genetically modified microbial cell is E. coli that has reduced (limited) or no growth on glucose, wherein the functionality of one or more endogenous proteins involved in glucose import and utilization, in said cell can be reduced or abolished. Preferably the proteins are selected from the group consisting of
i) glucose PTS complex components IICB^{Glc} (ptsG)
ii) beta-glucoside PTS complex components IIABC^{Bgl} (bglF)
iii) mannose PTS complex components—IICD^{Man}, (manX),
iv) N-acetylglucosamine PTS complex components IIABC^{Nag} (nagE)
v) maltose/maltodextrin transport system (malX)
vi) galactose/glucose high-affinity ABC transporter components (mglC);
vii) galactose permease (galP); and/or
viii) glucokinase (glk).

In embodiments described herein at least one of the genetically modified cells has reduced or abolished activity of at least one PTS-dependent sugar transport system selected from the group consisting of:
i) glucose PTS complex components IICB^{Glc};
ii) beta-glucoside PTS complex components—IIABC^{Bgl};
iii) mannose PTS complex components—IICD^{Man};
iv) N-acetylglucosamine PTS complex components—IIABC^{Nag}; and
v) Maltose/maltodextrin PTS complex—IICB^{malX}
vi) sorbitol PTS complex IICB^{slr}

Preferably, at least the ptsG gene of the glucose PTS complex components IICB^{Glc} is deleted in a bacteria, such as E. coli, that has no or limited growth on glucose.

### Glycerol Transport and Utilization

In embodiments described herein, a cell that grow on glycerol has at least one glycerol transport system. The glycerol transport system may be selected from the glycerol facilitator (glpF) or glycerol/H⁺-symporter (stl1).

In other embodiments reduction or prevention of import of glycerol into the cytosol of a microorganism can be achieved by mutating or deleting one or more sequences encoding proteins that affect the glycerol import capacity, such as deletion or mutation of nucleic acid sequences encoding glycerol facilitator (glpF) or glycerol/H⁺-symporter (stl1).

To utilize the glycerol to make energy the cell phosphorylates the glycerol once it has entered the cytosol to glycerol-3-phosphate, the phosphorylation is conducted by the glycerol kinase (glpK). Blocking the formation of glycerol-3-phosphate may therefore also serve to prevent the cell to utilize glycerol as a carbon source for growth. More information on glycerol utilization in various bacteria can be found in the review by Lin Ann. Rev. Microbial. 1976 30:535-78.

In embodiments the genetically modified cell has a reduced or no growth on glycerol wherein the functionality of one or more endogenous proteins involved in glycerol import and utilization, in said cell can be reduced or abolished. Preferably the reduction or abolished activity of the proteins is achieved by deletion or mutation of one or more nucleic acid sequences encoding a protein selected from the group consisting of glpF (e.g., Uniprot accession nr, or functional variants thereof), stl1 (e.g., Uniprot accession nr, or functional variants thereof) and glpK (e.g., Uniprot accession nr, or functional variants thereof).

In the event that it is desired that a bacteria, such as E. coli, has reduced or no growth on glycerol the functionality of the glycerol transporter protein, also known as the glycerol facilitator (glpF) is preferably reduced or abolished, e.g. by mutating or deleting the glpF gene in said cell. In addition, the activity of the glycerol kinase may be reduced or abolished, e.g. by mutating or deleting the glpK gene in said cell.

In embodiments of the method described herein one of the genetically modified cell has are reduced or abolished functionality of the proteins involved in glucose and/or glycerol import and utilization by full or partial inactivating one or more of the gene genes selected from the group consisting of ptsG, bglF, manX, nagE, malX, mglC, glk and glpF.

### Sucrose Transport and Utilization

In embodiments described herein, a cell that grows on sucrose has at least one sucrose transport system. The sucrose transport system may be the PTS-dependent sucrose (suc) utilization system. Alternatively, cells can grow on sucrose by having active sucrose invertase or sucrose hydrolase proteins in the outer membrane or the periplasmic membrane (if present), which are capable of cleaving sucrose to glucose and fructose which can then be taken up by the cell via relevant fructose and glucose transport systems (see for example WO 2013/087884).

In embodiments the genetically modified microbial cell capable of growing on sucrose comprises one or more nucleic acid sequences encoding a PTS-dependent sucrose utilization system. The PTS-dependent sucrose utilization system can for example be encoded by scrY, scrA, scrB and optionally scrR (see for example WO2015/197082), where the gene scrA codes for the sucrose transport protein Enzyme IIScr (e.g., SEQ ID NO: 98 or ncbi sequence ID: CAA40658.1 or functional variants thereof) that provides intracellular sucrose-6-phosphate from extracellular sucrose via an active transport through the cell membrane and the concomitant phosphorylation. The sucrose specific ScrY porin (e.g., SEQ ID NO: 97 or ncbi sequence ID: CAA40657.1 or functional variants thereof encoded by scrY) facilitate the sucrose diffusion through the outer membrane. The ScrB invertase enzyme (e.g., SEQ ID NO: 99 or ncbi sequence ID: WP_000056853.1 or functional variants thereof encoded by scrB) splits the accumulated sucrose-6-phosphate by hydrolysis to glucose-6-phosphate and fructose. The scrR encodes the Lad family DNA-binding transcriptional regulator (e.g., SEQ ID NO: 100 or ncbi sequence ID: WP_000851062.1 or functional variants thereof).

The E. coli csc PTS dependent sucrose system is described in WO2015/150328 expressed from the cscABKR gene cluster (SEQ ID NO: 110) encoding; sucrose permase (e.g. cscB with UniProt accession nr P30000.1 or functional variants thereof), fructokinase (e.g., csck with GenBank accession nr EDV65567.1 or functional variants thereof), sucrose hydrolase (e.g. cscA with NCBI accession nr WP_175214520.1 or functional variants thereof), and a transcriptional repressor (e.g. cscR with GenBank accession nr. AJA27326.1 or functional variants thereof).

Alternatively, the genetically modified microbial cell capable of growing on sucrose comprises a nucleic acid encoding a sucrose invertase or sucrose hydrolase enabling the assimilation of sucrose by said cell. The sucrose invertase may for example be a glycoside hydrolase and a sucrose-6-phosphate hydrolase (e.g., SacC_Agal with the GeneBank ID: WP_103853210.1 or SEQ ID NO: 111, or a functional variant thereof) or a beta-fructofuranosidase (e.g., Bff with GeneBank ID: BAD18121.1 or SEQ ID NO: 112, or a functional variant thereof). Since the sucrose hydrolase or sucrose invertase will convert the sucrose to glucose and fructose, e.g., in the periplasmic space of the cell, the cell should preferably be able to grow on either glucose or fructose, meaning that in a two strain system the other genetically modified cell should have no or limited growth on fructose and/or glucose.

In embodiments where the carbon source is sucrose, and its assimilation is enabled by a sucrose invertase or sucrose hydrolase the other cell in a two-strain system should preferably grow on glycerol or galactose.

In embodiments the genetically modified cell has a reduced or no growth on sucrose wherein the functionality of one or more endogenous proteins involved in sucrose import and utilization, in said cell can be reduced or abolished. Preferably the reduction or abolished activity of the proteins is achieved by deletion or mutation of one or more nucleic acid sequences selected from the group encoding proteins in the PTS-dependent sucrose utilization system, e.g., by mutating or deleting the cscABKR-gene cluster (SEQ ID NO: 110, or functional variants thereof), if present and functional in the cell. In particular deletion or mutation of the sucrose permease gene, such as cscB (e.g., Uniprot accession nr P30000.1, or functional variants thereof) or scrY (e.g., Uniprot accession nr B1LQA1, or functional variants thereof) is relevant if a strain is to show no or reduced grow on sucrose. Many non-pathogenic E. coli cells have lost the ability to grow on sucrose and it is therefore often not necessary to mutate the cell to prevent it from growing on sucrose since it no longer has the ability to do so.

### Galactose Transport and Utilization

In embodiments described herein, a cell that grow on galactose has at least one galactose transport system. The galactose transport system may be selected from the galactose:H+ symporter GalP, the galactose/glucose ABC transporter (mglABC) system, the PTSLac (lacFE) system and/or the sodium/glucose transporter family (sglT).

Galactose imported into the cell via GalP, mglABC or sglT is converted into galactose-1-phosphate (Gal1P) via galactose kinase which is in turn metabolized via the Leloir pathway (galMKTE) to alpha-glucose-1-phosphate (G1P).

Galactose imported into the cell via PTSLac (lacFE) system is converted into Galactose-6-phosphate (Gal6P) and further metabolized to triose phosphates by the Tag6P pathway (lacABCD).

A cell growing on galactose preferably also have a functional galactose kinase (galK), functional Leloir pathway and/or Tag6P pathway.

In other embodiments reduction or prevention of import of galactose into the cytosol of a microorganism can be achieved by mutating or deleting one or more sequences encoding proteins that affect the galactose import capacity, such as deletion or mutation of nucleic acid sequences galactose:H+ symporter GalP, the galactose/glucose ABC transporter (mglABC) system, sodium/glucose transporter family (sglT) or the PTSLac (lacFE) system.

To utilize the galactose to make energy the cell phosphorylates the galactose to gal1P or gal6P. Mutating, deleting of blocking the enzymes converting gal to gal1P or gal6P may therefore also serve to prevent the cell to utilize galactose as a carbon source for growth.

In embodiments the genetically modified cell has a reduced or no growth on galactose wherein the functionality of one or more endogenous proteins involved in galactose import and utilization, in said cell can be reduced or abolished. Preferably the reduction or abolished activity of the proteins is achieved by deletion or mutation of one or more nucleic acid sequences selected from the group encoding galP (e.g., Uniprot accession nr P0AEP1, or functional variants thereof), mglC (e.g., Uniprot accession nr P23200, or functional variants thereof), lacF (e.g., Uniprot accession nr P24400 or functional variants thereof), galK (e.g., Uniprot accession nr P0A6T3, or functional variants thereof) and/or sglT (e.g., Uniprot accession nr P96169, or functional variants thereof).

### Fructose Transport and Utilization

In embodiments described herein, a cell that grow on fructose has at least one fructose transport system. The fructose transport system may be selected from the fructose PTS complex components IIABC^{Fru}, glucose PTS complex components IICB^{Glc}, the fructose transporter FruP.

Fructose imported into the cell via is converted into fructose-1-phosphate (fru1P) or fructose-6-phosphate (fru6P) via fructose kinases.

In other embodiments reduction or prevention of import of fructose into the cytosol of a microorganism can be achieved by mutating or deleting one or more sequences encoding proteins that affect the fructose import capacity, such as deletion or mutation of nucleic acid sequences encoding components of the fructose PTS complex components IIABC^{Fru}, glucose PTS complex components IICB^{Glc}, the fructose transporter FruP.

To utilize the fructose to make energy the cell phosphorylates the fructose to fru1P or fru6P. Mutating, deleting of blocking the enzymes converting fructose to fru1P or fru6P may therefore also serve to prevent the cell to utilize fructose as a carbon source for growth.

In embodiments the genetically modified cell has a reduced or no growth on fructose wherein the functionality of one or more endogenous proteins involved in fructose import and utilization, in said cell can be reduced or abolished. Preferably the reduction or abolished activity of the proteins is achieved by deletion or mutation of one or more nucleic acid sequences selected from the group encoding fruA (e.g., Uniprot accession nr P20966, or functional variants thereof), ptsG (e.g., Uniprot accession nr P69786, or functional variants thereof), or FruP (e.g., Uniprot accession nr F4TKS5 or functional variants thereof).

### Maltose Transport and Utilization

In embodiments described herein, a cell that grow on maltose has at least one maltose transport system.

On such maltose transport system is the MalFGK ABC superfamily transport system which transports maltose across the cytoplasmic membrane of Escherichia coli. The MalFGK transport system is a heterotetrameric complex comprised of integral membrane proteins MalF and MalG, which associate with two units of the peripheral membrane protein MalK which possess ATP binding properties and hence may provide energy to the maltose permease encoded by malF and malG.

Alternatively, the maltose transport system may be selected from the maltose/maltodextrin PTS complex-IICB^{mal} (e.g., Uniprot accession nr P19642, or functional variants thereof), encoded by malX. The PTS enzyme-II protein encoded by malX is capable of recognizing both glucose and maltose as substrates.

In embodiments where the maltose/maltodextrin PTS complex is used for growth on maltose of one strain it is referred that the second strain has no or limited growth on glucose as well as on maltose.

Maltose imported into the cell is converted into glucose via amylomaltase (e.g., UniProt accession nr. P15977.2 or functional variants thereof) encoded by malQ or an alternative maltase from another species. The glucose is in turn phosphorylated as described in the “glucose transport” section above.

In other embodiments reduction or prevention of import of maltose into the cytosol of a microorganism can be achieved by mutating or deleting one or more sequences encoding proteins that affect the maltose import capacity, such as deletion or mutation of nucleic acid sequences encoding components of the MalFGK ABC superfamily transport system or the maltose PTS complex components IICB^{mal} Furthermore, deletion of glucokinase (glk, e.g., Uniprot accession nr P0A6V8, or functional variants thereof) will prevent utilization of maltose as carbon source since the glucose needs to be phosphorylated to be converted into energy by the cell.

In embodiments the genetically modified cell has a reduced or no growth on maltose wherein the functionality of one or more endogenous proteins involved in maltose import and utilization, in said cell can be reduced or abolished. Preferably the reduction or abolished activity of the proteins is achieved by deletion or mutation of one or more nucleic acid sequences selected from the group encoding MalF (e.g., UniProt accession nr P02916.1 or functional variants thereof), MalG (e.g., GenBank: AAC77002.1 or functional variants thereof), MalK (e.g., UniProt accession nr. P02916.1 or functional variants thereof), PTS complex—IICB^{mal} (e.g., Uniprot accession nr P19642, or functional variants thereof) encoded by malX and/or glucose kinase (e.g., Uniprot accession nr P0A6V8, or functional variants thereof) encoded by glk.

### Arabinose Transport and Utilization

In embodiments described herein, a cell that grow on arabinose has at least one arabinose transport system.

On such arabinose transport system is the AraFGH arabinose transporter which is a member of the ATP Binding Cassette (ABC) transporter superfamily. The AraF is the periplasmic binding protein (e.g, UniProt accession nr. P02924 or functional variants thereof), AraH is the membrane component (e.g, UniProt accession nr. P0AE26 or functional variants thereof) and AraG is the ATP-binding component of this ABC transporter (e.g. UniProt accession nr. P0AAF3 or functional variants thereof)

Alternatively, the arabinose transport system may be selected from the arabinose-proton symporter AraE (e.g. UniProt accession nr. P0AE24 or P96710 or functional variants thereof).

In embodiments the genetically modified cell has a reduced or no growth on arabinose wherein the functionality of one or more endogenous proteins involved in arabinose import and utilization, in said cell can be reduced or abolished. For example, by prevention of import of arabinose into the cytosol of a microorganism by mutating or deleting one or more sequences encoding proteins that affect the arabinose import capacity, such as deletion or mutation of nucleic acid sequences encoding components of the AraFGH ABC superfamily transport system or the maltose arabinose-proton symporter AraE.

### Sorbitol Transport and Utilization

In embodiments described herein, a cell that grow on sorbitol has at least one sorbitol transport system.

The transport of sorbitol into procaryotic cells is facilitated by a phosphoenolpyruvate-dependent phosphotransferase system (PTS). The sorbitol-specific Enzyme IIB and IIC (EIIBC^{srl}) components, are responsible for binding to sorbitol and initiating its transport into the cell, this enzyme is encoded by srlA (e.g., Uniprot accession nr P56579 or O32333 or functional variants of these) and srl E, (e.g., Uniprot accession nr P56580 or O32332 or functional variants of these) respectively. As part of the PTS process, the incoming sorbitol molecule is simultaneously phosphorylated by sorbitol kinase (EIIA^{srl}) encoded by the gene srlB (e.g., Uniprot accession nr P05706 or A5I7D9 or functional variants of these).

In embodiments where the sorbitol PTS complex is used for growth on sorbitol of one strain it is referred that the second strain has no or limited growth on glucose as well as on maltose.

In embodiments the genetically modified cell has a reduced or no growth on sorbitol wherein the functionality of one or more endogenous proteins involved in sorbitol import and utilization, in said cell can be reduced or abolished. For example, by prevention of import of sorbitol into the cytosol of a microorganism by mutating or deleting one or more sequences encoding proteins that affect the sorbitol PTS system.

Two-Strain System with Intermediate Oligosaccharide Uptake

In this application of the two-strain system the second strain is designed to produce an oligosaccharide by using the product produced from the first strain as initial substate for a glycosylation reaction to produce the second oligosaccharide. The intermediate dependent two-strain system applies the features of the two-strain system with the addition that the second genetically modified cell is capable of importing the disaccharide or oligosaccharide produced by the first genetically modified cell, in this way the first oligosaccharide serves as an intermediate oligosaccharide and acceptor in the production of the second oligosaccharide produced by the second genetically modified cell (illustrated in FIG. 1).

A second aspect described herein is a method for producing an oligosaccharide having at least three, such as at least four monosaccharide units, said method comprising the steps of co-culturing a first and a second genetically modified microbial cell in a culture medium, wherein,
a) the first genetically modified microbial cell is capable of producing a first disaccharide or a first oligosaccharide of at least three monosaccharide units and wherein said first genetically modified cell
   i) is capable of growing on a first carbon source while showing limited or no growth on a second carbon source, and
   ii) comprises one or more recombinant nucleic acid sequences encoding at least one glycosyltransferase; and
   iii) comprises at least one pathway to produce an activated sugar nucleotide from the first carbon source; and
   iv) is capable of exporting said first oligosaccharide into the culture medium; and
b) the second genetically modified microbial cell is capable of internalizing the first oligosaccharide of at least three monosaccharide units and wherein the second genetically modified cell
   i) is capable of growing on the second carbon source while showing limited or no growth on the first carbon source, and
   ii) comprises one or more recombinant nucleic acid sequences encoding at least one glycosyltransferase, and
   iii) comprises a biosynthetic pathway to produce an activated sugar nucleotide from the second carbon source
wherein the second genetically modified microbial cell produces the oligosaccharide having at least three, such as at least four monosaccharide units.

In embodiments the first genetically modified microbial cell produces an intermediate disaccharide or oligosaccharide of three, four or five monosaccharide units. Preferably, the disaccharide or oligosaccharide produced by the first genetically modified cell is selected from the group consisting of LacNAc, LNB, Lewis A, Lewis X, 2′FL, 3FL LNT-II, LNT, LNnT, LNFP-I, LST-c or LST-a.

In embodiments the first genetically modified cell producing an intermediate disaccharide or oligosaccharide comprises at least one glycosyltransferase selected from the group consisting of β-1,3-N-acetyl-glucosaminyltransferase, beta-1,3-galactosyltransferase, beta-1,4-galactosyltransferase, alpha-1,2-fucosyltransferase, alpha-1,3-fucosyltransferase, alpha-1,3/4-fucosyltransferase and alpha-2,3-sialyltransferase.

In some embodiments the first genetically modified cell producing an intermediate oligosaccharide comprises at least two glycosyltransferases. Preferably, such a cell produces LNT or LNnT, In some embodiments the first genetically modified cell producing an intermediate oligosaccharide comprises at least three glycosyltransferases. Preferably, such a cell produces LNFP-I, LST-c or LST-a.

In further embodiments the second genetically modified cell is capable of importing the disaccharide or oligosaccharide of three, four or five monosaccharide units produced by the first genetically modified cell. The disaccharides lactose, LacNAc and LNB can be imported by a lactose permease. To enable the import of oligosaccharides, the second genetically modified cell may additionally be modified such that it comprises at least one recombinant nucleic acid sequence and/or a cluster of recombinant nucleic acid sequences encoding a transporter protein and/or a cluster of proteins capable of importing the intermediate oligosaccharide produced by the first genetically modified cell.

Lactose permease (LacY) is known in its wild-type form to transport the disaccharide lactose from the cell exterior into the E. coli cell. The wild type lactose permease my also import 2′FL, LNB and LAcNAc, and can therefore serve as the importer protein in the second genetically modified cell when the first genetically modified cell produces 2′FL, LNB or LacNAc. It may however be preferred that the lactose permease is modified to have increased affinity for 2′FL and potentially 3FL or LNT-II over lactose.

Mutated variants of LacY have been described to be capable of transporting the trisaccharide maltotriose (Olsen et al 1993 J Bacteriol. 175(19):6269-75). In the present disclosure these mutants are described as potential importers of trisaccharides (acceptor oligosaccharides/HMO precursor molecules) of relevance in the HMO production, e.g., 2-fucosyllactose (2′FL), 3-fucosyllactose (3FL), lacto-N-triose (LNT-II).

The second genetically modified cell according to the present invention may comprise a recombinant nucleic acid sequence encoding a transporter protein capable of importing an intermediate (acceptor) oligosaccharide of at least three monosaccharide units, produced by the first genetically modified cell, into said cell, wherein said transporter protein is a mutated lactose permease (LacY) as shown in table 1.

TABLE 1
List of exemplary mutants of the E. coli DH1
K12 lactose permease LacY (SEQ ID NO: 1) that could
be useful for the import of 2′FL, 3FL or LNT-II

| | | | Wild-type | Mutant |
|---|---|---|---|---|
| Name | Mutation | Position | amino acid | amino acid |
| mut1 | Y236N | 236 | Tyr | Asn |
| mut2 | Y236H | 236 | Tyr | His |
| mut3 | S306T | 306 | Ser | Thr |
| mut4 | A177V | 177 | Ala | Val |
| mut5 | H322N | 322 | His | Asn |
| Mut6 | I303F | 303 | Ile | Phe |
| mut6 | Y236H, | 236 | Tyr | His |
| | S306T | 306 | Ser | Thr |
| mut7 | A177V, | 177 | Ala | Val |
| | Y236H | 236 | Tyr | His |
| mut8 | A177V, | 177 | Ala | Val |
| | I303F | 303 | Ile | Phe |
| mut9 | A177V, | 177 | Ala | Val |
| | H322N | 322 | His | Asn |
| mut10 | A177V, | 177 | Ala | Val |
| | S306T | 306 | Ser | Thr |
| mut11 | A177V, | 177 | Ala | Val |
| | Y236N, | 236 | Tyr | Asn |
| | S306T | 306 | Ser | Thr |

In preferred embodiments the lactose permease variant of table 1 have higher affinity for 2′FL, 3FL and/or LNT-II compared to lactose.

This may in particular be an advantage if the first genetically modified cell needs to internalize lactose to produce 2′FL, 3FL or LNT-II, otherwise the second genetically modified cell will also take up lactose which may also serve as a substrate (acceptor) in the second genetically modified cell and thereby result in undesired by-product formation.

In embodiments where the intermediate oligosaccharide produced by the first genetically modified cell is not 2′FL, LacNAc or LNB, it is preferred that the second strain does not contain any functional endogenous lactose permease. Endogenous lactose permeases can either be deleted or their function can be abolished by point mutations such as stop codons or truncations. The lack of a functional lactose permease in the second genetically modified strain will prevent undesired glycosylation of lactose by the glycosyl transferases in the second strain, thereby reducing by-product formation.

Typically, by-product oligosaccharides or by-product HMOs are i) the oligosaccharide or HMO precursor(s) which are modified further within the cell to produce the oligosaccharide or HMO of interest (product HMO/oligosaccharide) or ii) further decoration of the desired product oligosaccharide. In some embodiments, it may be desired to produce the product HMO/oligosaccharide in abundant amounts and by-product HMOs/oligosaccharides in minor amounts. Abundant amounts of the oligosaccharide/HMO of interest is for example at least 20%, such as at least 30%, such as at least 50%, such as at least 60%, such as at least 75%, such as at least 80%, such as at least 85%, such as at least 90% of the total amount of oligosaccharide/HMO produced. The term most abundant oligosaccharide/HMO in relation to what is produced by a genetically modified cell indicates that it is the most prevalent oligosaccharide/HMO in the culture broth/cells if the cell is fermented alone. Examples of by-products in the HMO production can for example be 2′FL and/or 3FL in the production of DFL, or the LNT-II in the formation of LNT and LNnT, wherein both cases not all of the precursor oligosaccharide is decorated in the second glycosylation reaction.

An alternative way to avoid that the second cell takes up lactose added to the medium as substrate for the first cell, would be to engineer the first cell to produce its own lactose (see for example parschat et al. 2020 ACS Synth. Biol. 9:2784-27969). In embodiments the first genetically modified microbial cell comprises a beta-1,4-galactosyltransferase allowing galactosylation of a glucose monosaccharide to intracellularly generate lactose and wherein the glucokinase activity, converting glucose into glucose-6-phosphate, in said cell is reduced or abolished. Preferably, a lactose producing cell does not use glucose as the carbon source for growth, since this would require conversion of glucose to glucose-6-phosphate, leaving less free glucose to be converted to lactose. More preferably a lactose producing cell uses sucrose or maltose as carbon source.

In embodiments the first genetically modified cell is capable of producing LNB, LacNAc, Lewis A, Lewis X, 2′FL, 3FL, LNT-II, LNT, LNnT LNFP-I or LST-a without the addition of lactose to the medium.

In embodiments where the first genetically modified cell produces an oligosaccharide of four or five monosaccharide units alternative transporter molecules may be needed in the second genetically modified cell, in particular transporters capable of internalizing LNT, LNnT, LNFP-I or LST-a are desired.

The second genetically modified cell according to the present invention may comprise a recombinant nucleic acid or a cluster of recombinant nucleic acid sequences encoding a transporter protein and/or a cluster of proteins capable of importing an intermediate (acceptor) oligosaccharide produced by the first genetically modified cell of at least three or four monosaccharide units into said cell.

Importer proteins with the potential to import oligosaccharides of three or four monosaccharides or more have been identified in Gram-positive (Gram+) bacteria, and in particular in members of the Bifidobacterium, Roseburia and Eubacterium species.

Table 2 shows MFS-transporter proteins of gram-positive origin and ABC-transporter protein clusters of gram-positive origin capable of importing an acceptor oligosaccharide of at least three or four monosaccharide units into a cell. The term transporter and importer may be used interchangeably.

The intermediate oligosaccharide produced by the first genetically engineered cell is preferably a precursor for a more complex HMO and can act as an acceptor oligosaccharide when imported into the second genetically modified cell. In table 2 it is indicated which intermediate/acceptor oligosaccharide the transporter is expected to import into the cell.

TABLE 2
ABC- and MFS-transporters from gram-positive bacteria with an indication
of the precursor oligosaccharide the transporter is expected to import.
The ABC transporters are composed of three to four genes. For ease of
reference each transporter has been given a transporter ID (TP ID)

| | | | | | Expected | |
|---|---|---|---|---|---|---|
| TP | TP | | | SEQ | oligosaccharide | |
| ID | type | Gene Name | GenBank ID | ID NO: | transport | Origin |
| 1 | MFS | Blon_0247 | ACJ51375.1 | 2 | LNnT | B. infantis ATCC |
| | | | | | | 15697 = JCM 1222 |
| 2 | MFS | Blon_0431 | ACJ51548.1 | 6 | oligosaccharide | B. infantis ATCC |
| | | | | | | 15697 = JCM 1222 |
| 3 | MFS | Blon_0788 | ACJ51891.1 | 7 | oligosaccharide | B. infantis ATCC |
| | | | | | | 15697 = JCM 1222 |
| 13 | MFS | Blon_0962 | ACJ52061.1 | 12 | Fucosylated-HMO | B. infantis ATCC |
| | | | | | LNT-II | 15697 = JCM 1222 |
| 14 | MFS | Blon_2307 | ACJ53365.1 | 19 | Fucosylated-HMO | B. infantis ATCC |
| | | | | | | 15697 = JCM 1222 |
| 4 | MFS | Blon_2400 | ACJ53455.1 | 27 | oligosaccharide | B. infantis ATCC |
| | | | | | | 15697 = JCM 1222 |
| 5 | ABC | Blon_2341 | ACJ53399.1 | 20 | LN(n)T | B. infantis ATCC |
| | | | | | | 15697 = JCM 1222 |
| | | Blon_2342 | ACJ53400.1 | 21 | | B. infantis ATCC |
| | | | | | | 15697 = JCM 1222 |
| | | Blon_2343 | ACJ53401.1 | 22 | | B. infantis ATCC |
| | | | | | | 15697 = JCM 1222 |
| | | Blon_2344 | ACJ53402.1 | 23 | | B. infantis ATCC |
| | | | | | | 15697 = JCM 1222 |
| 6 | ABC | Blon_2345 | ACJ53403.1 | 24 | LN(n)T | B. infantis ATCC |
| | | | | | | 15697 = JCM 1222 |
| | | Blon_2346 | ACJ53404.1 | 25 | | B. infantis ATCC |
| | | | | | | 15697 = JCM 1222 |
| | | Blon_2347 | ACJ53405.1 | 26 | | B. infantis ATCC |
| | | | | | | 15697 = JCM 1222 |
| 15 | ABC | Blon_0341/ | ACJ51465.1/ | 3 | 2′-FL, 3-FL, DFL, | B. infantis ATCC |
| | | Blon_2204 | ACJ53264.1 | 18 | LNFP-I | 15697 = JCM 1222 |
| | | Blon_0342/ | ACJ51466.1/ | 4 | | B. infantis ATCC |
| | | Blon_2203 | ACJ53263.1 | 17 | | 15697 = JCM 1222 |
| | | Blon_2202 | ACJ53262.1 | 16 | | B. infantis ATCC |
| | | | | | | 15697 = JCM 1222 |
| 16 | ABC | Blon_0341/ | ACJ51465.1/ | 3 | 2′-FL, 3-FL, DFL, | B. infantis ATCC |
| | | Blon_2204 | ACJ53264.1 | 18 | LNFP-I | 15697 = JCM 1222 |
| | | Blon_0342/ | ACJ51466.1/ | 4 | | B. infantis ATCC |
| | | Blon_2203 | ACJ53263.1 | 17 | | 15697 = JCM 1222 |
| | | Blon_0343 | ACJ51467.1 | 5 | | B. infantis ATCC |
| | | | | | | 15697 = JCM 1222 |
| 7 | ABC | Blon_0883 | ACJ51983.1 | 8 | LN(n)T, Lewis a, | B. infantis ATCC |
| | | | | | type 1 H- | 15697 = JCM 1222 |
| | | Blon_0884 | ACJ51984.1 | 9 | trisaccharide | B. infantis ATCC |
| | | | | | | 15697 = JCM 1222 |
| | | Blon_0885 | ACJ51985.1 | 10 | | B. infantis ATCC |
| | | | | | | 15697 = JCM 1222 |
| | | Blon_0886 | ACJ51986.1 | 11 | | B. infantis ATCC |
| | | | | | | 15697 = JCM 1222 |
| 8 | ABC | Blon_2177 | ACJ53238.1 | 15 | LNB, LN(n)T, | B. infantis ATCC |
| | | | | | LNO, LNH, sialyl | 15697 = JCM 1222 |
| | | Blon_2176 | ACJ53237.1 | 14 | LNT | B. infantis ATCC |
| | | | | | | 15697 = JCM 1222 |
| | | Blon_2175 | ACJ53236.1 | 13 | | B. infantis ATCC |
| | | | | | | 15697 = JCM 1222 |
| 9 | ABC | RHOM_04095 | AEN95941.1 | 35 | LNT-II, LNT | R. hominis A2-183 |
| | | RHOM_04100 | AEN95942.1 | 36 | | R. hominis A2-183 |
| | | RHOM_04105 | AEN95943.1 | 37 | | R. hominis A2-183 |
| 10 | ABC | HMPREF0373_{—} | ERK42291.1 | 38 | LNT | E. ramulus ATCC |
| | | 02960 | | | | 29099 |
| | | HMPREF0373_{—} | ERK42292.1 | 39 | | E. ramulus ATCC |
| | | 02961 | | | | 29099 |
| | | HMPREF0373_{—} | ERK42293.1 | 40 | | E. ramulus ATCC |
| | | 02962 | | | | 29099 |
| 17 | ABC | BBKW_1838/ | BAQ29973.1/ | 44 | 2-′FL, 3-FL, DFL, | B. kashiwanohense |
| | | BKAS_1807 | KFI63925.1 | 43 | LNFP-I (FL-2) | JCM 15439 |
| | | BBKW_1839/ | BAQ29974.1/ | 45 | | B. kashiwanohense |
| | | BKAS_1806 | KFI63924.1 | 42 | | JCM 15439 |
| | | BBKW_1840/ | BAQ29975.1/ | 46 | | B. kashiwanohense |
| | | BKAS_1805 | KFI63923.1 | 41 | | JCM 15439 |
| 18 | ABC | BBPC_1775 | BAR04453.1 | 47 | 2′-FL, 3-FL, DFL, | B. |
| | | | | | LNFP-I, LNT-II | pseudocatenulatum |
| | | | | | | JCM 1200 |
| | | BBPC_1776 | BAR04454.1 | 48 | | B. |
| | | | | | | pseudocatenulatum |
| | | | | | | JCM 1200 |
| | | BBPC_1777 | BAR04455.1 | 49 | | B. |
| | | | | | | pseudocatenulatum |
| | | | | | | JCM 1200 |
| 11 | ABC | BBR_0527/IntP1 | ABE95224.1 | 28 | LNT-II, LN(n)T | B. breve UCC2003 |
| | | BBR_0528/IntP2 | ABE95225.1 | 29 | | B. breve UCC2003 |
| | | BBR_0530/IntS | ABE95226.1 | 30 | | B. breve UCC2003 |
| | | BBR_0531 | ABE95228.1 | 50 | | B. breve UCC2003 |
| 12 | ABC | BBR_1554/nahS | ABE96225.1 | 31 | LNnT | B. breve UCC2003 |
| | | BBR_1558 | ABE96228.1 | 32 | | B. breve UCC2003 |
| | | BBR_1559 | ABE96229.1 | 33 | | B. breve UCC2003 |
| | | BBR_1560 | ABE96230.1 | 34 | | B. breve UCC2003 |

Typically, the second genetically modified cell lacks enzymatic activity liable to degrade the acceptor oligosaccharide of at least three, four or five monosaccharide units imported into the cell.

Once the intermediate oligosaccharide produced by the first cell is imported into the second cell it can act as an acceptor molecule for further glycosylation by one or more selected glycosyl transferases.

In embodiments the second genetically modified cell comprises at least one glycosyltransferase selected from the group consisting of β-1,3-N-acetyl-glucosaminyltransferase, beta-1,3-galactosyltransferase, beta-1,4-galactosyltransferase, alpha-1,2-fucosyltransferase, alpha-1,3-fucosyltransferase, alpha-1,3/4-fucosyltransferase, alpha-1,4-fucosyltransferase, alpha-2,3-sialyltransferase, and alpha-2,6-sialyltransferase.

In embodiments the one or more oligosaccharides, such as HMOs, produced by the second genetically modified microbial cell has at least three, such as at least four monosaccharide units and are selected from the group consisting of Lewis A, Lewis X, sialyl-LacNAc, sialyl-LNB, sialyl-Lewis X, sialyl-Lewis A, Lewis B, Lewis Y, DFL, FSL, LNT, LNnT, LST-a, LST-c, LNFP-I, LNFP-II, LNFP-III, LNFP-V, LNFP-VI, LNDFH-I, LNDFH-II, LNDFH-III, LST-a, LST-b, LST-c, LST-d, DSLNT, pLNH, pLNnH, LNH, LNnH, (D)F-LNH-I, (D)F-LNH-11, (D)F-LNH-111, F-para-LNH-I, DF-para-LNH, DF-para-LNnH, TF-LNH, FLST b, FLST a, FLST-c, S-LNH, S-LNnH-I, FS-LNH, FS-LNnH-I, and DS-F-LNH-11 or a mixture of these.

Preferably, both the first and second genetically engineered cells express a transporter protein (exporter) that can export the disaccharide or oligosaccharide produced by said cell. Transporter proteins for oligosaccharide export are described in the corresponding section below. In addition to the heterologous transporter proteins endogenous transporter proteins located at the plasma membrane and/or outer membrane of the cell, such as porins may further assist with the desired export of either the precursor oligosaccharide from the first genetically modified cell or the final oligosaccharide produced by the second genetically modified cell.

One embodiment disclosed is a method for producing LNnT, said method comprising co-culturing
a. a first genetically modified microbial cell capable of growing on a first carbon source while showing limited or no growth on a second carbon source and which comprises
   i a recombinant nucleic acid sequence encoding a beta-1,3-N-acetylglucosaminyltransferase, and
   ii optionally a recombinant nucleic acid sequence encoding a MFS transporter capable of exporting LNT-II into the extracellular medium, and
b. a second genetically modified microbial cell capable of growing on the second carbon source while showing limited or no growth on the first carbon source and which comprises
   i a recombinant nucleic acid sequence encoding a protein or protein complex capable of importing the oligosaccharide produced by said first genetically modified microbial cell is recombinant and encodes a transporter protein capable of importing LNT-II, selected from the group consisting of a mutant lacY-transporter or a potential LNT-II transporter selected from table 2,
   ii a recombinant nucleic acid sequences encoding a beta-1,4-galactosyltransferase,
   iii optionally a recombinant nucleic acid encoding the MFS transporter vag, and
c. harvesting the LNnT produced in the co-culture, and
wherein the method produces significantly less LNT-II and/or minimal or no pLNnH by-product compared to LNnT produced by a single cell.

In preferred embodiments the second genetically modified cell is not capable of importing lactose, in that it for example is deficient of a functional lactose permease.

One embodiment disclosed is a method for producing LNT, said method comprising co-culturing
a. a first genetically modified microbial cell capable of growing on a first carbon source while showing limited or no growth on a second carbon source and which comprises
   i a recombinant nucleic acid sequence encoding a beta-1,3-N-acetylglucosaminyltransferase, and
   ii optionally a recombinant nucleic acid sequence encoding a MFS transporter capable of exporting LNT-II into the extracellular medium, and
b. a second genetically modified microbial cell capable of growing on the second carbon source while showing limited or no growth on the first carbon source and which comprises
   i a recombinant nucleic acid sequence(s) encoding a protein or protein complex capable of importing the oligosaccharide produced by said first genetically modified microbial cell is recombinant and encodes a transporter protein capable of importing LNT-II, selected from the group consisting of a mutant lacY-transporter or a potential LNT-II transporter selected from table 2,
   ii a recombinant nucleic acid sequences encoding a beta-1,3-galactosyltransferase
   iii optionally, a recombinant nucleic acid encoding the MFS selected from nec or YberC,
c. harvesting the LNT produced in the co-culture,
wherein the method produces significantly less LNT-II and/or minimal or no pLNH2 by-product compared to LNT produced by a single cell.

In preferred embodiments the second genetically modified cell is not capable of importing lactose, in that it for example is deficient of a functional lactose permease.

An example of an LNT-II MFS exporter is the putative metabolite transport protein YjhB from E. coli.

One embodiment disclosed is a method for producing LNFP-II, LNFP-VI and/or LNDFH-III, said method comprising co-culturing
a. a first genetically modified microbial cell capable of growing on a first carbon source while showing limited or no growth on a second carbon source and which comprises,
   i. a recombinant nucleic acid sequence encoding a beta-1,3-N-acetylglucosaminyl transferase, and
   ii. a recombinant nucleic acid sequence encoding a beta-1,4-galactosyltransferase, and
   iii. optionally a recombinant nucleic acid sequence encoding a MFS transporter capable of exporting LNnT into the extracellular medium and
b. a second genetically modified microbial cell capable of growing on the second carbon source while showing limited or no growth on the first carbon source and which comprises
   i. a nucleic acid sequence(s) encoding a protein or protein complex capable of importing the oligosaccharide produced by said first genetically modified microbial cell is recombinant and encodes a transporter protein capable of importing LNnT, and
   ii. the recombinant nucleic acid sequences encoding a glycosyltransferase encodes an alpha-1,3-fucosyltransferase or alpha-1,3/4-fucosyltransferase, and
c. harvesting the LNFP-III, LNFP-VI and/or LNDFH-III produced in the co-culture,
wherein the method produces significantly less LNnT, pLNnH and LNT-II by-product as well as fucosylated derivatives thereof compared to LNFP-III, LNFP-VI and/or LNDFH-III produced by a single cell.

In preferred embodiments the second genetically modified cell is not capable of importing lactose, in that it for example is deficient of a functional lactose permease.

### Two-Strain Hybrid System

A further application of the two-strain system is highly suitable for the production of complex oligosaccharides, such as for example complex fucosylated and/or sialylated oligosaccharides or neutral non-fucosylated oligosaccharides of at least five such as at least six monosaccharide units. It may however also be used to produce shorter oligosaccharides of three or four monosaccharide units.

Currently, complex oligosaccharides are primarily produced using in vitro enzymatic synthesis. The in vitro enzymatic process relies on the use of a donor oligosaccharide (HMO) and an acceptor oligosaccharide (HMO) which are catalyzed by an enzyme with transglycosidase activity to produce a third oligosaccharide (complex HMO), however due to the nature of enzymatic reactions the oligosaccharides (HMOs) produced by this route will always be a mixture of the donor, the acceptor and the third oligosaccharide (complex HMO) as well as a side-product moiety released from the donor substrate (the leaving group e.g. lactose) due to the equilibrium of the enzymatic reaction (see for example WO2012/156897, WO2012/156898 and WO2016/063262). Furthermore, the enzymatic process utilizes separately produced and purified donor and acceptor substrates which increases the cost of the process. Additionally, in cases where side-product released from the donor substrate is lactose, extensive purification is required to remove this large amount of lactose which is undesired. Furthermore, the conventional enzymatic process generally does not allow for the removal of one of the substrates due to the kinetic equilibrium in the reaction.

Bioproduction systems using in vivo fermentation of the HMOs is currently the preferred mode of production for the smaller fucosylated and sialylated and neutral core HMOs (for review see Bych et al 2019, Current Opinion in Biotechnology 56:130-137). However, with more complex HMOs the fermentation route may face challenges in terms of exporting the HMOs from the cell into the medium, which is necessary to achieve high yields in industrial scale.

The two-strain hybrid process described herein combines the best properties from the in vivo bioproduction system and the in vitro enzymatic production system of oligosaccharides, such as HMOs, by combining these into a hybrid production system combining a fermentation step and an enzymatic step in the same vessel. As illustrated in FIG. 2, two strains can be co-cultured as described in the section “two-strain system” above and the oligosaccharides, such as HMOs, produced by the two strains can be reacted catalyzed by a transglycosidase in the culture medium to form a third complex oligosaccharide.

The realization that an enzymatic transglycosylation reaction, allowing the formation of a complex HMO, could effectively be conducted in the culture medium of a running fermentation process, as illustrated Examples 2, was rather surprising. In the hybrid process the conditions are dictated by the fermentation conditions (e.g., temperature, pH, oxygen, carbon dioxide, stirring etc.) and the reaction environment is significantly more complex with multiple substrates and metabolites in the fermentation broth and potentially including proteases released from the cells, compared to the conventional in vitro enzymatic processes illustrated in Examples 2 where there are only two initial substrates (acceptor and donor) and the transglycosidase enzyme. To the best of our knowledge, this is the first time an enzymatic reaction has been used to synthesize a larger molecule from two smaller molecules simultaneously produced in a running fermentation.

The hybrid production system described in the present disclosure comprises a co-culturing fermentation step and an enzymatic step which can be conducted in the same vessel as the co-culture.

In the two-stain hybrid process a first genetically modified cell produces a disaccharide or preferably a first oligosaccharide which is secreted/exported into the culture medium of the fermentation. The first oligosaccharide can either act as a donor oligosaccharide, or it can act as the acceptor oligosaccharide in the subsequent transglycosylation reaction occurring in the culture medium. The first genetically modified cell is preferably engineered such that it effectively can produce the first oligosaccharide/HMO and export it into the culture medium. Furthermore, the process comprises a second genetically modified cell producing a second oligosaccharide which is preferably secreted/exported into the culture medium of the fermentation. The second oligosaccharide can either act as a donor oligosaccharide, or it can act as the acceptor oligosaccharide in the subsequent transglycosylation reaction occurring in the culture medium. In embodiments where the complex oligosaccharide produced by the process is a fucosylated and/or sialylated oligosaccharide the donor oligosaccharide contains a fucosyl- or sialyl-residue. In preferred embodiments the donor oligosaccharide is fucosyllactose or sialyllactose.

In embodiments where the complex oligosaccharide produced by the process is neutral core oligosaccharide (non-fucosylated) the donor oligosaccharide of at least six monosaccharide units, such as hexa-, octa-, deca or dodeca-oligosaccharides, the donor oligosaccharide is preferably LNT or LNnT.

In the production of complex HMOs, the first and second oligosaccharides are preferably HMOs. The genetically modified cells are engineered such that they effectively can produce the oligosaccharide/HMO and release or export it into the culture medium while grown in the same vessel. Preferably, the co-culturing of the cells is controlled by their ability to grow on different carbon sources which the other cell does not have significant (limited) growth on. Once the cells have produced some of the first and the second oligosaccharide/HMO they are reacted catalyzed by a transglycosidase enzyme in the culture medium, to form a third complex oligosaccharide/HMO, such as a sialylated and/or fucosylated and/or hexa- or octa-neutral core oligosaccharide/HMO.

A third aspect described herein relates to a method for producing a oligosaccharide of at least three monosaccharide units or a complex oligosaccharide of at least four, such as at least five monosaccharide units from a donor oligosaccharide and an acceptor oligosaccharide produced by a first and a second genetically modified cell, said method comprising the steps of:
a) co-culturing a first and second genetically modified cell in a culture medium, wherein
   i) the first genetically modified microbial cell is capable of producing a disaccharide or a first oligosaccharide of at least three monosaccharide units and wherein the genetically modified cell
      comprises one or more recombinant nucleic acid sequences encoding at least one glycosyltransferase; and
      comprises at least one pathway to produce a nucleotide-activated sugar from the first carbon source; and
      is preferably capable of exporting said first oligosaccharide into the culture medium; and
   ii) the second genetically modified microbial cell is capable of producing a second oligosaccharide of at least three monosaccharide units and wherein the genetically modified cell
      comprises one or more recombinant nucleic acid sequences encoding at least one glycosyltransferase, and
      comprises a biosynthetic pathway for making said activated sugar nucleotide from the second carbon source;
      is preferably capable of exporting said second oligosaccharide into the culture medium, and
b) making an enzyme with transglycosidase activity available in the culture medium, and
c) incubating the first oligosaccharide or disaccharide with the second oligosaccharide and the transglycosidase enzyme in the culture medium in which the first oligosaccharide or disaccharide and second oligosaccharides are reacted to form a third oligosaccharide of at least three, such as four, such as five monosaccharide units.

Embodiments described herein relates to a method for producing a sialylated and/or a fucosylated oligosaccharide of at least four monosaccharide units from a donor oligosaccharide and an acceptor oligosaccharide produced by a first and a second genetically modified cell, said method comprising the steps of:
a) co-culturing a first and second genetically modified cell in a culture medium, wherein
   i) the first genetically modified microbial cell is capable of producing a disaccharide or a first oligosaccharide of at least three monosaccharide units and wherein the genetically modified cell
      is capable of growing on a first carbon source while showing limited or no growth on a second carbon source, and
      comprises one or more recombinant nucleic acid sequences encoding at least one glycosyltransferase; and
      comprises at least one pathway to produce a nucleotide-activated sugar from the first carbon source; and
      is preferably capable of exporting said first oligosaccharide into the culture medium; and
   ii) the second genetically modified microbial cell is capable of producing a second oligosaccharide of at least three monosaccharide units and wherein the genetically modified cell
      is capable of growing on the second carbon source while showing limited or no growth on the first carbon source, and
      comprises one or more recombinant nucleic acid sequences encoding at least one glycosyltransferase, and
      comprises a biosynthetic pathway for making said activated sugar nucleotide from the second carbon source;
      is preferably capable of exporting said second oligosaccharide into the culture medium wherein either the first or the second oligosaccharide is a fucosyl or sialyl donor oligosaccharide and the other oligosaccharide or disaccharide is an acceptor oligosaccharide or acceptor disaccharide, and
b) making an enzyme with transglycosidase activity available in the culture medium, wherein the enzyme with transglycosidase activity is
   i) a transfucosidase if the donor oligosaccharide is a fucosylated oligosaccharide, or
   ii) a transsialidase if the donor oligosaccharide is a sialylated oligosaccharide, and
c) incubating the first oligosaccharide or disaccharide, with the second oligosaccharide, and the transglycosidase enzyme in the culture medium in which the first and second oligosaccharides are produced to form a third sialylated and/or fucosylated oligosaccharide of at least three, such as at least four monosaccharide units.

Embodiments described herein relates to a method for producing a neutral core oligosaccharide of at least six monosaccharide units from a donor oligosaccharide and an acceptor oligosaccharide produced by a first and a second genetically modified cell, said method comprising the steps of:
a) co-culturing a first and second genetically modified cell in a culture medium, wherein
   i) the first genetically modified microbial cell is capable of producing a first oligosaccharide of at least four monosaccharide units, such as LNT or LNnT and wherein the genetically modified cell
      is capable of growing on a first carbon source while showing limited or no growth on a second carbon source, and
      comprises one or more recombinant nucleic acid sequences encoding at least one glycosyltransferase; and
      comprises at least one pathway to produce a nucleotide-activated sugar from the first carbon source; and
      is preferably capable of exporting said first oligosaccharide into the culture medium; and
   ii) the second genetically modified microbial cell is capable of producing a second oligosaccharide of at least four monosaccharide units which is different from the first oligosaccharide, such as LNT, LNnT, pLNnH or pLNH2, and wherein the genetically modified cell
      is capable of growing on the second carbon source while showing limited or no growth on the first carbon source, and
      comprises one or more recombinant nucleic acid sequences encoding at least one glycosyltransferase, and
      comprises a biosynthetic pathway for making said activated sugar nucleotide from the second carbon source;
      is preferably capable of exporting said second oligosaccharide into the culture medium wherein either the first oligosaccharide is donor oligosaccharide and the second oligosaccharide is an acceptor oligosaccharide, and
b) making an enzyme with transglycosidase activity available in the culture medium, wherein the enzyme with transglycosidase activity is
   i) a transfucosidase if the donor oligosaccharide is a fucosylated oligosaccharide, or
   ii) a transsialidase if the donor oligosaccharide is a sialylated oligosaccharide, and
c) incubating the first oligosaccharide or disaccharide, with the second oligosaccharide, and the transglycosidase enzyme in the culture medium in which the first and second oligosaccharides are produced to form a third sialylated and/or fucosylated oligosaccharide of at least three, such as at least four monosaccharide units.

In the contexts of the hybrid production method the term “first oligosaccharide” or “first HMO” or “first disaccharide” or just “disaccharide” refers to the oligosaccharide or disaccharide produced in-situ by the first genetically modified cell, and which constitute the first substrate in the enzymatic (transglycosidase) step of the hybrid process In embodiments where the “first” disaccharide is produced from the first genetically modified strain it preferably acts as an acceptor. Furthermore the “first” disaccharide is not lactose and preferably it is lacto-N-biose (LNB) or N-acetyllactosamine (LacNAc). The term, “second oligosaccharide” or “second HMO” refers to the oligosaccharide produced in-situ by the second genetically modified cell and which constitute the second substrate in the enzymatic (transglycosidase) step of the hybrid process. The first and the second oligosaccharides are different and capable of acting as a donor substrate and acceptor substrate in a transglycosylation process. In embodiments where the oligosaccharide produced in the process is a Lewis A or Lewis X based oligosaccharide or a complex fucosylated and/or sialylated oligosaccharide the donor oligosaccharide contains a fucosyl- or sialyl-residue.

When the first and the second oligosaccharides are reacted with the transglycosidase a third oligosaccharide and a side-product (leaving group) is produced. The leaving group is re-cycled by at least one of the genetically modified cells to produce more of the oligosaccharide produced by said cell. The third oligosaccharide is preferably the desired complex oligosaccharide of the process, it may however also act as an intermediate oligosaccharide for a second transglycosidase reaction which produces a fourth oligosaccharide which is the desired complex oligosaccharide of the process. If the hybrid process comprises two enzymatic steps it can either be a two-step enzymatic process catalyzed by the same transglycosidase or by transglycosidases with different activity, e.g., one is a transfucosidase and the other is a transsialidase depending on their selectivity. However, in the case of using two different transglycosidases the other substrate for the second transglycosidase might require to be supplied to the process.

The enzymatic transglycosidase reaction occurs in the culture medium and the first and the second oligosaccharides produced by the genetically modified cells therefore needs to be available in the culture medium before the reaction can take place. Preferably, the first and second oligosaccharides are exported out of their respective cells without affecting the survival of the cells. In alternative embodiments, the first and/or second oligosaccharide may be released (become available) into the culture medium by natural lysis of a portion of the cells during the fermentation, without stopping the culture from growing.

In embodiments, it is desired that the first genetically modified cell exports the first oligosaccharide produced by the cell into the culture medium and second genetically modified cell exports the second oligosaccharide produced by the cell into the culture medium to make them easily available for the transglycosidase reaction in the culture medium.

In embodiments, oligosaccharides, such as HMOs, which can advantageously be produced and exported to the culture medium by one of the genetically modified cells and which can serve as donor oligosaccharide in the transglycosylation reaction can be selected form the group consisting of Lewis A, Lewis X, 2′FL, 3FL, DFL, sialyl-lacNAc, sialyl-LNB, FSL, LNT, LNnT, LNFP-I, LST-a, 3′SLAcNAc, 3′SLNB, 3′SL and 6′SL.

In embodiments, oligosaccharides, such as HMOs, which can be produced by one of the genetically modified cells and serve as acceptor oligosaccharide in the transglycosylation reaction can be selected form the group consisting of LNB, LacNac, 2′FL, 3FL, 2′FLacNAc, 2′FLNB, Lewis A, Lewis X, LNT-II, LNT, LNnT, Para-LNnH, LNFP-I, LNFP-II, LNFP-II, LNFP-IV, LNFP-V, LNFP-VI, LST-a, LST-c and LST-d.

In embodiments, HMOs which can advantageously be produced and exported to the culture medium by one of the genetically modified cells and serve as acceptor oligosaccharide in the transglycosylation reaction can be selected form the group consisting of LNB, LacNAc, Lewis A, Lewis X, 2′FL, 3FL, LNT-II, LNT, LNnT, and LNFP-I.

The genetically modified cells are capable of producing the first oligosaccharide from a substrate which is preferably added to the culture medium and taken up by the cell to serve as the initial substrate for the production of the first oligosaccharide, e.g., HMO. In cases where the third oligosaccharide has a N-acetyllactoseamine (LacNAc) lacto-N-biose (LNB) at the reducing end the initial substrate may be selected from N-acetyllactoseamine (LacNAc) lacto-N-biose (LNB), which is then decorated in the cell in a similar way as lactose to produce for example Lewis A, Lewis X, 3′SLacNAc, 3′SLNB, 2′FLacNAc or 2′FLNB. For HMOs, lactose is most commonly used as the initial substrate, but LNT-II can potentially also serve as substrate for LNT or LNnT production or 2′FL or 3FL can serve as substrate for DFL production. In embodiments the substrate for the production of the first HMO can be selected from lactose, 2′FL, 3FL or LNT-II. Preferably, the initial substrate is selected from lactose or 2′FL. In a preferred embodiment the substrate for the production of the first and second oligosaccharide/HMO is lactose. As an alternative to adding the initial substrate for the production of the first and/or second oligosaccharide to the fermentation medium, the genetically modified cell may be further engineered to produce the initial substrate inside the cell (see for example WO2015/150328).

To achieve the in-situ recycling of the side-product, it is advantageous if at least one of the genetically modified cells is capable of internalizing the initial substrate used in the production of the first and/or second oligosaccharide.

In embodiments at least one of the genetically modified cells is capable of internalizing lactose, 2′FL, 3FL and/or LNT-II, depending on which compound is used as the initial substrate for making the first or second HMO. This initial substrate internalized by the cell(s) may correspond to the side-product produced by the transglycosidase reaction, which is thereby re-cycled. Preferably, at least one of the genetically modified cells uses lactose as the initial substrate, and if the side-product produced by the transglycosidase reaction is not lactose, lactose is fed to the culture during the fermentation. Also, in the embodiment where the fermentation is a co-culture and both cells use lactose as the initial substrate, lactose is preferably also feed to the culture during fermentation to avoid running out of the initial substrate.

In some embodiments, one of the genetically modified cells is preferably capable of internalizing lactose or 2′FL added to the culture medium, which at least one of the cells then utilizes for the production of the first or second oligosaccharide (e.g., HMO). Some microbial cells have endogenous lactose uptake systems, for example in the form of a lactose permease, which is also capable of importing 2′FL. Lactose permeases can also be genetically engineered into the cell either as a heterologous protein or as an additional recombinant copy of the native gene, if a higher lactose uptake is desired.

In preferred embodiments, both the first and the second genetically modified cells are capable of internalizing lactose as the initial substrate for the production of the first and second oligosaccharide, respectively.

In embodiments, the cultivation of the genetically modified cells is initiated in the presence of sufficient substrate(s) for the cells to produce the first and second oligosaccharide. In some embodiments, sufficient substrate(s) to produce the desired amount of the first and second oligosaccharide is present at the initiation of the cultivation, such that no additional substrate(s) is added to the culture medium after the initiation of the cultivation. In other embodiments, at least one of the substrates, such as lactose, to produce the first and/or second oligosaccharide is continuously fed to the culture during the fermentation, to secure that the cell(s) do not run out of substrate, this is in particular relevant if both cells use the same substrate. In embodiments the initial substrate for producing the first and/or second oligosaccharide can be selected independently from lactose, LacNAc, LNB, 2′FL, 3FL and LNT-II. In preferred embodiments the substrate for producing the first and second oligosaccharide is independently selected from lactose or 2′FL. Preferably, the substrate for producing the first and second oligosaccharide is identical for both strains, most preferred the substrate for producing the first and second oligosaccharide is lactose.

In alternative embodiments, the substrate(s) for producing the first and second oligosaccharide, such as lactose, is added to the culture medium when the initial carbon sources are consumed, thereby allowing initial growth of the first and second genetically modified cells before initiating the production of the first and second oligosaccharide. The substrate, such as lactose, can either be added as a single portion or be feed separately or together with one or both carbon sources.

In the co-culturing process described herein it may be desirable to control the ratios of the first and the second oligosaccharide such that the molar ratio of the first and second oligosaccharide are balanced to secure optimal formation of the third oligosaccharide. In one example the molar ratio of the first and second oligosaccharide is 1:1 (equimolar). In some embodiments, it may be advantages to have an excess of the second oligosaccharide over the first oligosaccharide, such as a 1.5:1-10:1 ratio of second oligosaccharide over first oligosaccharide, such as 1.5:1-5:1, such as 2:1, 3:1, 4:, 5:1, 6:1, 7:1, 8:1, 9:1, 10:1. For example in a two-strain hybrid approach to form a complex oligosaccharide, the affinity constants (Km's) of the enzyme for the donor and acceptor might not be the same. In order, to maximize the efficiency of the enzymatic reaction the donor and acceptor can therefore advantageously be supplied in a ratio that reflects the Km's. The ratios of the first and second oligosaccharides can be controlled by adapting the feeding rate of the first and the second carbon source. In particular towards the end of the fermentation it may be desired to reduce the amount of one of the oligosaccharides to secure less by-product in the final product obtained from the hybrid process. This can either be done by reducing the carbon source the strain producing the oligosaccharide on, or alternative reducing the substrate used to produce the oligosaccharide, if it is different from the substrate used to produce the other oligosaccharide.

In order to be able to reduce the levels of side-product (e.g., lactose) produced as the leaving group in the transglycosylation process (enzymatic process) of the two-strain hybrid process, at the end of the process it is desired that the feeding of the substrate for the cells to produce the first and second oligosaccharide (e.g., lactose) is stopped earlier than the end of the process. When lactose is formed together with the third complex oligosaccharide as a result the transglycosidase reaction at least one of the genetically modified cells will be able to internalize the lactose and thereby remove any lactose produced in the enzymatic process and convert it into additional first and/or second oligosaccharide. This, in addition to removing undesired lactose from the culture medium, also serves to push the equilibrium towards formation of additional third oligosaccharide/complex HMO, which therefore can be produced in higher ratios than either the first and/or second oligosaccharides or both as compared to the conventional enzymatic process. If the feed of lactose is stopped based on a design to reach a full conversion of the acceptor oligosaccharide/HMO or higher molar ratio of the third complex HMO to the acceptor oligosaccharide/HMO, it is preferred that the transglycosidase enzyme is deactivated at the same time as the lactose feed is reduced to prevent the accumulation of a leaving group of the enzymatic step (e.g. lactose) by the side hydrolytic activity of the enzyme that triggers the reverse reaction and to maintain the designed product composition. This can be achieved by a change in pH, temperature or addition of a protease.

In other embodiments, the side-product from the transglycosidase reaction may be an oligosaccharide, such as an oligosaccharide with three monosaccharide units, such as an HMO. In this case one of the genetically modified cells are preferably engineered such that it can take up the side-product oligosaccharide and use it as substrate for the production of the first or second oligosaccharide going into the transglycosylation process in the culture medium. For example, one of the genetically modified cells produces DFL which serves as fucosyl donor in the transfructosylation reaction which then results in 2′FL as side-product (leaving group). The 2′FL is then taken up by the DFL producing cell which is capable of using the 2′FL as substrate for the production of DFL. In this case the other strain most likely will need a different substrate, such as lactose for producing the other oligosaccharide for the transglycosidase reaction.

In embodiments, the weight % of the third oligosaccharide/complex HMO exceeds the weight % of the donor oligosaccharide at the end of the process. In some embodiments, the ratio between the third oligosaccharide and the first and/or second oligosaccharide is above 1.5:1, such as above 2:1, such as 5:1. By above a certain ratio, is meant that the first number indicated in the ratio can be the number indicated or larger than the indicated number.

In the two-strain hybrid process described herein, the transglycosidase enzyme mediating the transglycosylation of the acceptor oligosaccharide with the donor oligosaccharide is available in the culture medium of the fermentation.

In embodiments the transglycosidase enzyme is expressed from a recombinant nucleic acid in one of the genetically modified cells producing the first or second oligosaccharide. Alternatively, it may be expressed in a third strain growing on a third carbon source or on one of the carbon sources of the first or the second strain, since it may not be needed to balance the expression of the enzyme. The enzyme may become available in the culture medium by natural lysis of a portion of the cells during the fermentation, without stopping the culture from growing. It is advantageous that the enzyme is exported to the culture medium. The export of the transglycosidase enzyme can for example be facilitated using appropriate signal peptides.

For expression in E. coli the signal peptide can for example be selected from one of the following well known signal peptides

TABLE 34
suitable signal peptides for expression of heterologous transglycosidase in E. coli.

| Signal | | | |
|---|---|---|---|
| sequence | Origin | Amino Acid Sequence | SEQ ID NO: |
| pelB | Erwinia carotovora | MKYLLPTAAAGLLLLAAQPAMA | 114 |
| ompA | Escherichia coli | MKKTAIAIAVALAGFATVAQA | 115 |
| ompC | Escherichia coli | MKVKVLSLLVPALLVAGAANA | 127 |
| ompG | Escherichia coli | MKKLLPCTALVMCAGMACAQA | 128 |
| ompT | Escherichia coli | MRAKLLGIVLTTPIAISSFA | 129 |
| dsbA | Escherichia coli | MKKIWLALAGLVLAFSASA | 116 |
| lamB | Escherichia coli | MMITLRKLPLAVAVAAGVMSAQAMA | 117 |
| phoA | Escherichia coli | MKQSTIALALLPLLFTPVTKA | 118 |
| PhoE | Escherichia coli | MKKSTLALVVMGIVASASVQA | 130 |
| malE | Escherichia coli | MKIKTGARILALSALTTMMFSASALA | 119 |
| mglB | Escherichia coli | MNKKVLTLSAVMASMLFGAAAHA | 120 |
| CueO | Escherichia coli | MKKIWLALAGLVLAFSASA | 131 |
| dsbC | Escherichia coli | MKKGFMLFTLLAAFSGFAQA | 121 |
| TorT | Escherichia coli | MRVLLFLLLSLFMLPAFS | 132 |
| MdoD | Escherichia coli | MDRRRFIKGSMAMAAVCGTSGIASLFSQAAFA | 133 |
| FhuD | Escherichia coli | MSGLPLISRRRLLTAMALSPLLWQMNTAHA | 134 |
| LivK | Escherichia coli | MKRNAKTIIAGMIALAISHTAMA | 135 |
| STII | Escherichia coli | MKKNIAFLLASMFVFSIATNAYA | 136 |
| MmAp | Escherichia coli | MKKNIIAGCLFSLFSLSALA | 137 |
| GIII | Escherichia coli | MKKLLFAIPLVVPFYSHS | 138 |
| Nsp2 | Escherichia coli | MKKITAAAGLLLLAAFSASA | 139 |
| Nsp4 | Escherichia coli | MKKITAAAGLLLLAAQPAMA | 146 |
| Pac | Escherichia coli | MKNRNRMIVNCVTASLMYYWSLPALA | 140 |
| SacC-AgaI1 | Avibacterium gallinarum | MIIFNEGKYKSLYAAEQGEL | 141 |
| SacC-AgaI2 | Avibacterium gallinarum | MIIFNEGKYKSLYAAEQGELEKIAQTVAQD | 142 |
| SacC-AgaI4 | Avibacterium gallinarum | MIIFNEGKYKSLYAAEQGELEKIAQTVAQDQDF | 143 |
| | | RPVYHLAPPTGLLNDPN | |
| YcdO | Escherichia coli | MTINFRRNALQLSVAALFSSAFMANA | 144 |
| YraI | Escherichia coli | MSKRTFAVILTLLCSFCIGQALA | 145 |

In other embodiments the transglycosidase enzyme is added exogenously to the culture medium during the cultivation of the genetically modified cell. When the enzyme is added exogenously it is preferably sterile filtered prior to the addition to avoid contamination of the culture. The transglycosidase is added to the hybrid process in sufficient activity to mediate the transglycosylation of the acceptor oligosaccharide with the donor oligosaccharide. If the activity of the enzyme is reduced during the fermentation, it may be advantages to add the enzyme when sufficient substrate has been produced by the genetically modified cell for it not to be rate limiting for the process. It may also be possible to add enzyme more than one time to the cultivation process.

In some embodiments the transglycosidase is added to the culture medium at a time point when the genetically modified cells have converted at least 50% of the initial lactose into the first and second oligosaccharides, such as at least 75% of the initial lactose, such as at least 85% of the initial lactose, such as at least 90% of the initial lactose, such as between 95% and 100% of the initial lactose. It is advantageous to allow formation of a sufficient amount of first and second oligosaccharide before initiating the transglycosylation reaction to make sure that the substrates for the enzymatic process do not become rate limiting and the unconverted lactose does not inhibit the reaction.

At the end of the fermentation, it is desired to deactivate the transglycosidase enzyme to avoid the shift of equilibrium of the transglycosidase reaction once the formation of the first oligosaccharide stops due to the cessation of the carbon source feed which provides energy and carbon for the genetically modified cells that is required for the continued recycling of lactose. Preferably, the deactivation is done before the cells are harvested or immediately after the harvest. Non-limiting examples of deactivation the transglycosidase can be selected from i) heating the fermentation broth to a temperature that denatures the enzyme, ii) adding a protease to the culture broth at the end of fermentation to hydrolyze the enzyme or iii) change the pH of the culture such that it is outside the activity range of the enzyme or denatures the enzyme. If heating is used for deactivation, it is preferred that the broth is heated to at least 60° C., such as at least 70° C., such as at least 80° C., such as at least 90° C., such as at least 95° C. for at least 5 minutes, such as at least 10 minutes, such as at least 15 minutes. If a protease is used to deactivate the enzyme, it is preferably added in a sufficient activity to hydrolyze all the enzyme at least 10 min, such as at least 20 min such as at least 30 min prior to the harvest of the cells. If a change in pH is used to deactivate the enzyme, it is preferred to decrease the pH to below 5, preferably to between 3 to 5 such as between 3.5 to 4.5.

### Transglycosidase

Glycoside hydrolases are carbohydrate-processing enzymes in nature. Apart from hydrolysis activities, some of them also exhibit high transglycosylation activities, also called transglycosidases that catalyze the transfer of a sugar moiety between different glycosides and/or oligosaccharides.

In the contexts of the two-strain hybrid method described herein it is advantageous if the transglycosidase enzyme has as low a hydrolytic activity as possible. The hydrolytic activity of for example a transsialidase results in hydrolyses of the donor oligosaccharide, in case of 3′SL the hydrolysis reaction produces lactose and sialic acid, as well as hydrolysis of the third oligosaccharide to form the acceptor and sialic acid, e.g., in case of FSL to 3FL and sialic acid. The hydrolytic activity of for example a transfucosidase results in hydrolyses of the donor oligosaccharide, in case of 2′FL or 3FL the hydrolysis reaction produces lactose and fucose, and hydrolysis of the third oligosaccharide to form the acceptor and fucose, e.g., in case of LNFP-III to LNnT and fucose. Typically, the hydrolytic activity of the enzyme is suppressed with sufficient acceptor substrate relative to the donor substrate. However, since in the two-strain hybrid process the side hydrolytic product (e.g., lactose) is recycled back to produce the first and/or second oligosaccharide in the genetically modified cells the effect on the third HMO product formation is very low, since the transglycosidase activity will then regenerate the third oligosaccharide. Hydrolytic and transfructosylation activity of transfucosidase enzyme can for example be measured as described in Zeuner et al. 2018 Enzyme and Microbial Technology 115:37-44. Similar assays can be used for transsialidases, substituting 3FL with 3′SL or 6′SL. However, with respect to the functionality in the two-strain hybrid process described herein it is preferable to compare the hydrolytic activity of potential transglycosidases in the actual process and then assess the amount of fucose or sialic acid generated by the respective enzymes. It is desirable to use a transfucosidase that produces as little fucose as possible in the hybrid method described herein. Likewise, it is desired to use a transsialidase that produces as little sialic acid as possible in the hybrid method described herein. It is desirable to use a trans-lacto-N-biosidase (LnbX) that produces as little lacto-N-biose as possible. Fucose, sialic acid and lacto-N-biose levels can for example be measured by HPLC, or alternative methods known by the person skilled in the art.

In the hybrid two-strain method described herein the transglycosidase is supplied to the two-strain culture in an amount sufficient to mediate the transglycosylation of an acceptor oligosaccharide with a sugar moiety from a donor oligosaccharide. In the hybrid two-strain method where the sugar moiety that is transferred is either a fucosyl or a sialyl moiety the enzyme with transglycosidase activity is a transfucosidase or a transsialidase, respectively.

In the hybrid two-strain method where the sugar moiety that is transferred is either a galactose or a N-acetylglucosamine (GlcNAc) moiety the enzyme with transglycosidase activity is a trans-β-galactosidase or a trans-lacto-N-biosidase or β-N-acetylglucosaminidase, respectively.

In embodiments, the transglycosidase enzyme is selected from the group consisting of alpha-1,2-tranfucosidase, alpha-1,3-transfucosidase, alpha-1,3/4-transfucosidase, alpha-2,3-transialidase, alpha-2,6-transsialidase, trans-lacto-N-biosidase, β-N-acetylglucosaminidase and trans-β-galactosidase.

It is advantageous if the transfucosidase is capable of using a fucosyllactose (e.g., 2′FL, 3FL or DFL) as fucosyl donor and a second oligosaccharide as acceptor. Likewise, it is advantageous if the transsialidase is capable of using a sialyllactose (3′SL or 6′SL) as sialyl donor and a second oligosaccharide as acceptor. It is advantageous if the β-1,3-N-acetylglucosaminidase is capable of using LNT-II as GlcNAc donor and a second oligosaccharide as acceptor. It is advantageous if the β-1,3-galactosidase is capable of using LNT as galactose donor and a second oligosaccharide as acceptor. It is advantageous if the trans-lacto-N-biosidase is capable of using LNT, LNFP-I and LST-a as donor and a second oligosaccharide as acceptor.

In embodiments where the complex oligosaccharide of at least four or five monosaccharide units produced by the hybrid two-strain method described here is an HMO, the transglycosidase has substrate specificity for an oligosaccharide acceptor which preferably is an HMO containing at least three monosaccharide units, such as four, five, six or seven monosaccharide units. In embodiments, the complex oligosaccharide has at least five monosaccharide units and is a neutral non-fucosylated complex oligosaccharide, such as a neutral core HMO.

In embodiments where the sialylated and/or a fucosylated oligosaccharide of at least four monosaccharide units produced by the hybrid two-strain method described here, is an HMO the transfucosidase or transsialidase has substrate specificity for an oligosaccharide acceptor which preferably is an HMO containing at least three monosaccharide units, such as four, five, six or seven monosaccharide units.

In embodiments the transfucosidase and/or a transsialidase has substrate specificity for at least one acceptor disaccharide or oligosaccharide selected form the group consisting of LNB, LAcNAc, 2′FL, 3FL, Lewix A, Lewis X, 2′FLacNAc, 2′FLNB, LNT, LNnT, LNH, LNnH, para-LNH, para-LNnH, LNFP-I, LNFP-II, LNFP-III, LNFP-V, LNFP-VI, LSTa and LSTc.

In further embodiments the transfucosidase and/or a transsialidase has substrate specificity for at least one HMO acceptor oligosaccharide selected form the group consisting of 2′FL, 3FL, LNT, LNnT, LNH, LNnH, para-LNH, para-LNnH, LNFP-I, LNFP-II, LNFP-III, LNFP-V, LNFP-VI, LSTa and LSTc.

One embodiment of the application is a method for producing FSL comprising the steps of:
a) co-cultivating a first genetically modified cell growing on a first carbon source and producing 3FL and a second genetically modified cell growing on a second carbon source and producing 3′SL in a culture medium supplied with the first and second carbon source; and
b) making an enzyme with transsialidase activity available in the culture medium; and
c) incubating the 3FL, 3′SL and the transsialidase enzyme in the culture medium to form FSL and lactose; and
wherein the lactose is recycled by the first and second genetically modified cells to produce more 3FL and 3′SL.

Another embodiment of the application is a method for producing LNDFH-I comprising the steps of:
a) co-cultivating a first genetically modified cell growing on a first carbon source and producing 3FL and a second genetically modified cell growing on a second carbon source and producing LNFP-I in a culture medium supplied with the first and second carbon source; and
b) making an enzyme with transfucosidase activity available in the culture medium, and
c) incubating the 3FL, LNFP-I and the transfucosidase enzyme in the culture medium to form LNDFH-I and lactose, and
wherein the lactose is recycled by the first and second genetically modified cells to produce more 3FL and LNFP-I.

Another embodiment of the application is a method for producing LST-c comprising the steps of:
a) co-cultivating a first genetically modified cell growing on a first carbon source and producing 6′SL and a second genetically modified cell growing on a second carbon source and producing LNnT in a culture medium supplied with the first and second carbon source; and
b) making an enzyme with transsialidase activity available in the culture medium, and
c) incubating the 6′SL, LNnT and the transsialidase enzyme in the culture medium to form LST-c and lactose, and
wherein the lactose is recycled by the first and second genetically modified cells to produce more 6′SL and LNnT.

Another embodiment of the application is a method for producing LNFP-III comprising the steps of:
a) co-cultivating a first genetically modified cell growing on a first carbon source and producing 3FL and a second genetically modified cell growing on a second carbon source and producing LNnT in a culture medium supplied with the first and second carbon source; and
b) making an enzyme with transfucosidase activity available in the culture medium, and
c) incubating the 3FL, LNnT and the transfucosidase enzyme in the culture medium to form LNFP-III and lactose, and
wherein the lactose is recycled by the first and second genetically modified cells to produce more 3FL and LNnT.

Another embodiment of the application is a method for producing para-LNH comprising the steps of:
a) co-cultivating a first genetically modified cell growing on a first carbon source and producing LNT and a second genetically modified cell growing on a second carbon source and producing LNnT in a culture medium supplied with the first and second carbon source; and
b) making an enzyme with trans-lacto-N-biosidase activity available in the culture medium, and
c) incubating the LNT, LNnT and the trans-lacto-N-biosidase enzyme in the culture medium to form para-LNH and lactose, and
wherein the lactose is recycled by the first and second genetically modified cells to produce more LNT and LNnT.

To expand on the specific embodiments above specifying a method for producing a specific complex oligosaccharide, Table 3 below is a non-limiting list of sialylated and/or a fucosylated oligosaccharides of at least four monosaccharide units that can potentially be obtained using different transglycosidase activities with a fucosyllactose or a sialyllactose as donor oligosaccharide and second oligosaccharide as the acceptor oligosaccharide.

TABLE 3
Non-limiting examples of complex oligosaccharides obtainable using the two-strain hybrid process

| | | Second | | | Complex |
|---|---|---|---|---|---|
| First | | Oligosaccharide | | transglycosidase | Oligosaccharide |
| Oligosaccharide | Function | or disaccaride | Function | activity | produced |
| 2′FL | Donor | 3FL | Acceptor | α-1,2-transfucosidase | DFL |
| DFL | | LNT | | α-1,2-transfucosidase | LNFP-I |
| | | LNFP-II | | α-1,2-transfucosidase | LNDFH-I |
| | | Lewis-X | | α-1,2-transfucosidase | Lewis-Y |
| | | Lewis-A | | α-1,2-transfucosidase | Lewis-B |
| 3FL | Donor | LNT | Acceptor | α-1,3/4-transfucosidase | LNFP-II |
| DFL | | LNT | | α-1,3-transfucosidase | LNFP-V |
| | | | | (Glc specific) | |
| | | 2FL | | α-1,3-transfucosidase | DFL |
| | | LNnT | | α-1,3-transfucosidase | LNFP-III |
| | | | | (GlcNAc specific) | |
| | | LNnT | | α-1,3-transfucosidase | LNFP-IV |
| | | | | (Glc specific) | |
| | | LNFP-I | | α-1,4-transfucosidase | LNDFH-I |
| | | LNFP-V | | α-1,4-transfucosidase | LNDFH-II |
| | | LNFP-VI | | α-1,3-transfucosidase | LNDFH-III |
| | | | | (GlcNAc specific) | |
| | | LNFP-III | | α-1,3-transfucosidase | LNDFH-III |
| | | LNFP-II | | α-1,3-transfucosidase | LNDFH-II |
| | | LacNAc | | α-1,3/4-transfucosidase | Lewis-X |
| | | LNB | | α-1,3/4-transfucosidase | Lewis-A |
| | | Para- | | α-1,3-transfucosidase | DF-para-LNnH |
| | | LNnH | | (GlcNAc specific) | |
| 3′SL | Donor | LNFP-II | Acceptor | α-2,3-transsialidase | FLSTa (S-LNFP-II) |
| FSL | | 3FL | | α-2,3-transsialidase | FSL |
| | | LNT | | α-2,3-transsialidase | LSTa |
| | | LNFP-I | | α-2,3-transsialidase | FLSTa |
| | | LacNAc | | α-2,3-transsialidase | 3′SLacNAc |
| | | LNB | | α-2,3-transsialidase | 3′SLNB |
| 6′SL | Donor | LNnT | Acceptor | α-2,6-transsialidase | LSTc |
| | | LNFP-III | | α-2,6-transsialidase | FLSTc |
| | | LacNAc | | α-2,6-transsialidase | 6′SLN |
| | | LNFP-I | | α-2,6-transsialidase | FLSTb (S-LNFP-I) |
| | | | | (GlcNAc specific) | |
| | | LNT | | α-2,6-transsialidase | LSTb |
| | | | | (GlcNAc specific) | |
| | | LSTa | | α-2,6-transsialidase | DSLNT |
| | | | | (GlcNAc specific) | |
| | | LNB | | α-2,6-transsialidase | 6′SLNB |
| | | LacNAc | | α-2,6-transsialidase | 6′SLacNAc |
| LNT | Donor | LNnT | Acceptor | trans-lacto-N-biosidase | para-LNH |
| | | LNnT | | β-1,3-galactosidase | gal-LNnT |
| | | 3-FL | | trans-lacto-N-biosidase | LNFP-V |
| LNFP-I | Donor | LNnT | Acceptor | trans-lacto-N-biosidase | F-p-LNH |
| LST-a | Donor | LNnT | Acceptor | trans-lacto-N-biosidase | S-p-LNH |
| LNT-II | Donor | 3′FL | Acceptor | β-1,3-N- | GlcNAc(1-3)-3FL |
| | | | | acetylglucosaminidase | |
| 3′SLacNAc | Donor | Lewis-X | Acceptor | α-2,3-transsialidase | Sialyl-Lewis X |
| 3′SLNB | Donor | Lewis-A | Acceptor | α-2,3-transsialidase | Sialyl-Lewis A |

An example of a trans-lacto-N-biosidase from B. longum JCM1217 (LnbX, Sakamura et al. J. Biol. Chem. 288, 25194 (2013), GenBank nr. DAA64542) and its truncated functional analogs can be utilized to make linear lacto-N-biose containing oligosaccharides. In one embodiment variants with 70% identity to the sequence from amino acid position 45 to 625 of GenBank nr. DAA64542 with a mutation in at least at one or more of amino acid positions selected from 410, 416, 439 and 442, said amino acid numbering being according to GenBank nr. DAA64542 are used to transfer lacto-N-biose moieties from a donor oligosaccharide to an acceptor oligosaccharide. Advantages variants are described in PA202201151 where they are showed to function in an in vitro process generating pLNH from LNT and LNnT.

In one embodiment the trans-lacto-N-biosidase is comprises or consist of an amino acid sequence of SEQ ID NO: 122 or a functional variant thereof, in particular a variant where at position 410 Gly (G) is substituted by Trp, Tyr, Phe or His, preferably Trp; and/or position 416 Asp (D) is substituted by Asn or Gln, preferably Asn; and/or at position 439 Met (M) is substituted by Leu, Val or lie, preferably Leu, and/or at position 442 Asn (N) is substituted by Trp, Tyr, Phe or His, preferably Trp.

Non-limiting examples of relevant transsialidases and transfucosidases are shown in tables 4 and 5 below.

### Transsialidase

Enzymes having transsialidase activity and which are suitable for the purpose of making sialylated oligosaccharides with the two-strain hybrid process described herein, can be selected from sialidase and transsialidase enzymes.

Sialidases or neuraminidase (EC 3.2.1.18) and trans-sialidases (EC 2.4.1.-), both classify in the GH33 family as defined by the CAZY nomenclature (http://www.cazy.org), as enzymes with the ability of hydrolyzing the alpha-linkage of the terminal sialic acid (exo-α-sialidase), bound to galactose or glucose with an alpha-2,3 or an alpha-2,6 linkage, of various sialylglycoconjugates. The enzymes are found particularly in diverse virus families and bacteria, and also in protozoa, some invertebrates and mammals. Sialidases, are despite the hydrolytic activity, capable of acting as a catalyst for a transsialylation reaction due to their transsialidase activity with alpha-2,3 and/or alpha-2,6 selectivity.

In order to improve transsialidase activity of the sialidases, they may be subjected to alteration by various engineering techniques. Preferably, under the conditions in the hybrid two-strain method described herein, the formation of sialic acid is low. Preferably, the amount of sialic acid is below 5% of the total molar % of the donor oligosaccharide and the third oligosaccharide, more preferably below 3% of the total molar % of the donor oligosaccharide and the third oligosaccharide. WO2012/007588 describe a series of suitable transsialidases.

TABLE 4
Suitable transsialidases

| GI number in | | SEQ |
|---|---|---|
| GenBank Database | Organisms | ID NO: |
| gi\|213524659 | Bifidobacterium longum subsp. | 51 |
| gi\|213523006 | infantis ATCC 15697 | 52 |
| gi\|309252191 | Bifidobacterium bifidum S17 | 53 |
| gi\|309252190 | | 54 |
| gi\|310867437 | Bifidobacterium bifidum PRL2010 | 55 |
| gi\|310867438 | | 56 |
| gi\|224283484 | Bifidobacterium bifidum NCIMB 4 1171 | 57 |
| gi\|224283485 | | 58 |
| gi\|334283443 | Bifidobacterium bifidum JCM1254 | 59 |
| gi\|840708 | Trypanosoma cruzi | 60 |
| gi\|432485 | | 61 |
| — | Variant of gi\|432485 | 113 |
| gi\|267844791 | Photobacterium leiognathi | 87 |
| — | N-terminal truncated variant of | 88 |
| | gi\|267844791 | |

In embodiments the transsialidase is selected from the group of the suitable transsialidase enzymes in table 4 or a functional homologue thereof having an amino acid sequence of at least 70% identity, such as at least 80%, such as at least 85%, such as at least 90%, such at least 95% or even 97%, 98% or 99% identity compared to an individual transsialidase sequence in table 4.

In one embodiment the transfucosidase comprises or consist of an amino acid sequence of SEQ ID NO: 60, 91, 87, 88 or 113.

In embodiments where the transsialidase is added to the fermentation broth it is sterile filtered before it is introduced into the two-strain hybrid process. The transsialidase is added to the two-strain hybrid process in an activity sufficient to mediate the transsialylation of the acceptor oligosaccharide with the donor oligosaccharide.

In alternative embodiments, at least one of the genetically modified cells are further modified by introducing a heterologous nucleic acid which encodes a transsialidase. Preferably, the transsialidase is secreted/exported into the culture medium by the further genetically modified cell. The heterologous nucleic acid encoding the transsialidase may be expressed from an inducible promoter, such that the expression of the transsialidase is delayed compared to the formation of the oligosaccharide produced by the same cell. The advantage of having delayed expression of the transsialidase is that the first and/or second oligosaccharide will not become rate limiting in the enzymatic step of the two-strain hybrid process.

### Transfucosidases

Enzymes having transfucosidase activity and which are suitable for the purpose of making fucosylated oligosaccharides with the two-strain hybrid process described herein, can be selected from fucosidase and transfucosidase enzymes.

Alpha-L-fucosidases are classified according to EC 3.2.1.38 and EC 3.2.1.51 and belong to the glycoside hydrolases families 29 and 95 (GH29 and GH95) as defined by the CAZY nomenclature (http://www.cazy.org). The substrate specificity of the GH29 family is broad whereas that of the GH95 family has strict specificity to alpha-1,2-linked fucosyl residues. The GH29 family seems to be divided into two subfamilies. One subfamily typically has strict specificity towards alpha-1,3- and alpha-1,4-fucosidic linkages. The members of a further subfamily have broader specificity, covering two or three alpha-fucosyl linkages. Alpha-L-fucosidases generally hydrolyse the terminal fucosyl residue from glycans.

These enzymes are also capable to act as catalyst for a fucosylation reaction due to their transfructosylation activity and thus may be used in the context of the hybrid method described herein.

In order to improve transfucosidase activity of the fucosidases may be subjected to alteration by various engineering techniques. WO2016/063261 and Zeuner et al (2018 Enzyme and Microbial Technology 115:37-44) describes mutants of an alpha-1-34 transfucosidase from Bifidobacterium longum subsp. infants ATCC 15697 (NBI accession No. WP_012578573) or Bifidobacterium bifidum JCM 1254 (GenBank BAH80310.1), which have increased transfucosidase activity and reduced hydrolase activity Preferably, under the conditions in the hybrid two-strain method described herein, the formation of fucose is low. Preferably, the amount of fucose is below 5% of the total molar % of the donor oligosaccharide and the third oligosaccharide, more preferably below 3% of the total molar % of the donor oligosaccharide and the third oligosaccharide.

TABLE 5
Suitable transfucosidases

| GI number in | | SEQ |
|---|---|---|
| GenBank Database | Organisms | ID NO: |
| gi\|4980806 | Thermotoga maritima MSB8 | 62 |
| gi\|13816464 | Sulfolobus solfataricus P2 | 63 |
| gi\|34451973 | Bifidobacterium bifidum JCM 1254 | 64 |
| gi\|242345155 | Bifidobacterium bifidum JCM 1254 | 65 |
| — | Variant of gi\|242345155 | 66 |
| gi\|213524647 | Bifidobacterium longum subsp. | 67 |
| | infantis ATCC 15697 | |
| gi\|213522629 | Bifidobacterium longum subsp. | 68 |
| | infantis ATCC 15697 | |
| gi\|213522799 | Bifidobacterium longum subsp. | 69 |
| | infantis ATCC 15697 | |
| gi\|213524646 | Bifidobacterium longum subsp. | 70 |
| | Infantis ATCC 15697 | |
| gi\|501574167 | Bifidobacterium longum subsp. | 71 |
| | Infantis ATCC 15697 | |
| gi\|320457227 | Bifidobacterium longum subsp. | 72 |
| | infantis ATCC 15697 | |
| gi\|320457408 | Bifidobacterium longum subsp. | 73 |
| | infantis ATCC 15697 | |
| gi\|320459369 | Bifidobacterium longum subsp. | 74 |
| | infantis ATCC 15697 | |
| gi\|320459368 | Bifidobacterium longum subsp. | 75 |
| | infantis ATCC 15697 | |
| gi\|501574157 | Bifidobacterium longum | 76 |
| — | W135E, A174F Variant of gi\|501574157 | 77 |
| gi\|310867039 | Bifidobacterium bifidum PRL2010 | 78 |
| gi\|310865953 | Bifidobacterium bifidum PRL2010 | 79 |
| gi\|309251774 | Bifidobacterium bifidum S17 | 80 |
| gi\|296182927 | Bifidobacterium longum subsp | 81 |
| | longum JDM 301 | |
| gi\|283103603 | Bifidobacterium dentium Bd1 | 82 |
| gi\|190713109 | Lactobacillus casei BL23 | 83 |
| gi\|190713871 | Lactobacillus casei BL23 | 84 |
| gi\|190713978 | Lactobacillus casei BL23 | 85 |
| — | S168E, A174H, E413R Variant of | 86 |
| | gi\|501574157 | |
| — | W135F, A174N, N274A, E413R Variant | 123 |
| | of gi\|501574157 | |

In embodiments the transfucosidase is selected from the group of the suitable transfucosidase enzymes in table 5 or functional homologues thereof having an amino acid sequence of at least 70% identity, such as at least 80%, such as at least 85%, such as at least 90%, such at least 95% or even 97%, 98% or 99% identity compared to an individual transfucosidase sequence in table 5.

In embodiments the transfucosidase enzyme originates from Bifidobacterium bifidum or Bifidobacterium longum.

In one embodiment the transfucosidase comprises or consist of an amino acid sequence of SEQ ID NO: 66, 77, 86 or 123.

In embodiments the transfucosidase is added to the hybrid two-strain method it is sterile filtered before it is introduced into the two-strain hybrid process. The transfucosidase is added to the two-strain hybrid process in an activity sufficient to mediate the transfructosylation of the acceptor oligosaccharide with the donor oligosaccharide.

In alternative embodiments at least one of the genetically modified cells are further modified by introducing a heterologous nucleic acid which encodes a transfucosidase. Preferably, the transfucosidase is secreted/exported into the culture medium by the further genetically modified cell. The heterologous nucleic acid encoding the transfucosidase may be expressed from an inducible promoter, such that the expression of the transfucosidase is delayed compared to the formation of the oligosaccharide produced by the same cell. The advantage of having delayed expression of the transfucosidase is that the first and second oligosaccharides will not become rate limiting in the enzymatic step of the two-strain hybrid process.

### The Genetically Modified Cell

In the present context, the terms “a genetically modified cell” and “a genetically engineered cell” are used interchangeably. As used herein “a genetically modified cell” is a host cell whose genetic material has been altered by human intervention using a genetic engineering technique, such a technique is e.g., but not limited to transformation or transfection e.g., with a heterologous polynucleotide sequence, Crisper/Cas editing and/or random mutagenesis. In one embodiment the genetically engineered cell has been transformed or transfected with a recombinant nucleic acid sequence.

The genetic modifications can e.g., be selected from inclusion of glycosyltransferases, transglycosidases, and/or metabolic pathway engineering and inclusion of transporter proteins, including importer and exporters as described in the present application, all of which the skilled person will know how to combine into a genetically modified cell capable of producing the desired oligosaccharides/HMOs.

The genetically engineered cell is preferably a microbial cell, such as a prokaryotic cell or eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

### Host Cells

Regarding the bacterial host cells, there are, in principle, no limitations, they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be Erwinia herbicola (Pantoea agglomerans), Citrobacter freundii, Campylobacter sp, Corynebacterium sp., Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Corynebacterium glutamicum, Gluconobacter oxydans, Streptococcus thermophiles and Propionibacterium freudenreichii are also suitable bacterial species for the invention described herein. Also included as part of this invention are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a heterologous product are e.g., yeast cells of the genera Komagataella, Kluyveromyces, Yarrowia, Pichia, Saccaromyces, Schizosaccharomyces or Hansenula or from a filamentous fungus of the genera Aspergillus, Fusarium or Trichoderma. More specifically yeast cell species such as Komagataella phaffii, Kluyveromyces lactis, Yarrowia lipolytica, Pichia pastoris, and Saccaromyces cerevisiae or filamentous fungi species such as A. niger, A. nidulans, A. oryzae, F. solani, F. graminearum and T. reesei.

In one or more exemplary embodiments, the genetically engineered cell is selected from the group consisting of Escherichia coli, Corynebacterium glutamicum, Gluconobacter oxydans, Lactococcus lactis, Bacillus subtilis, Streptomyces lividans, Pichia pastoris and Saccharomyces cerevisiae.

In one or more exemplary embodiments, the genetically engineered cell is S. cerevisiae or P. pastoris.

In one or more exemplary embodiments, the genetically engineered cell is selected from the group consisting of Escherichia coli, Corynebacterium glutamicum, Gluconobacter oxydans, Lactococcus lactis, Bacillus subtilis, Streptomyces lividans.

In one or more exemplary embodiments, the genetically engineered cell is a gram-positive bacterium. In a further embodiment the gram-positive bacterium is selected from the group consisting of Bacillus subtilis, Corynebacterium glutamicum, Lactococcus lactis, Streptomyces lividans.

In one or more exemplary embodiments, the genetically engineered cell is Bacillus subtilis.

In one or more exemplary embodiments, the genetically engineered cell is Corynebacterium glutamicum.

In one or more exemplary embodiments, the genetically engineered cell is a gram-negative bacterium. In a further embodiment the gram-negative bacterium is selected from the group consisting of Escherichia coli and Gluconobacter oxydans.

In embodiments the first and second genetically modified microbial cell are selected from a yeast. In one embodiment one of the strains is selected from the group of yeast strains consisting of Yarrowia lipolytica, Pichia pastoris, and Saccharomyces cerevisiae and the other strain is selected from the group of yeast strains consisting of an Yarrowia lipolytica, Pichia pastoris, and Saccharomyces cerevisiae. It may be advantageous the yeast strains are of the same species.

In embodiments the first genetically modified microbial cell is selected from a bacterial and the and second genetically modified microbial cell is selected from a yeast. In one embodiment one of the strains is selected from the group of bacterial strains consisting of Bacillus subtilis, Corynebacterium glutamicum, Lactococcus lactis and Streptomyces lividans strain and the other strain is selected from the group of yeast strains consisting of an Yarrowia lipolytica, Pichia pastoris, and Saccharomyces cerevisiae strain.

In embodiments the first and second genetically modified microbial cell are selected from bacterial species. In one embodiment one of the strains is selected from the group of bacterial strains consisting of Bacillus subtilis, Corynebacterium glutamicum, Lactococcus lactis and Streptomyces lividans and the other strain is selected from the group of bacterial strains consisting of Bacillus subtilis, Corynebacterium glutamicum, Lactococcus lactis and Streptomyces lividans. In one embodiment one of the strains is an Escherichia coli strain and the other strain is a Bacillus subtilis or Corynebacterium glutamicum strain. It may be advantageous the bacterial strains are of the same species

It may be advantageous that the first and second genetically modified microbial cell are selected from the same species.

In one or more exemplary embodiments, the genetically engineered cell(s) is Escherichia coli. In one embodiment the first and second genetically modified microbial cell are Escherichia coli.

In one or more exemplary embodiments, the invention relates to a genetically engineered cell, wherein the cell is derived from the E. coli K-12 strain or DE3 strain.

### Glycosyltransferases

The genetically modified cells according to the present invention comprises at least one recombinant nucleic acid sequence encoding at least one glycosyltransferase capable of transferring a glycosyl residue from a glycosyl-donor to an acceptor oligosaccharide (substrate) to synthesize an oligosaccharide product, such as a human milk oligosaccharide product. The nucleic acid sequence encoding the one or more expressed glycosyltransferase(s) may be integrated into the genome (by chromosomal integration) of the genetically engineered cell, or alternatively, it may be comprised in a plasmid and expressed as plasmid-borne, as described in the present disclosure.

The genetically modified cell according to the present invention may comprise at least two recombinant nucleic acid sequences encoding two different glycosyltransferases capable of transferring a glycosyl residue from a glycosyl-donor to an acceptor oligosaccharide.

The one or more glycosyltransferase is preferably selected from the group of enzymes having the activity of an α-1,2-fucosyltransferase, α-1,3-fucosyltransferase, α-1,3/4-fucosyltransferase, α-1,4-fucosyltransferase α-2,3-sialyltransferase, α-2,6-sialyltransferase, β-1,3-N-acetylglucosaminyltransferase, β-1,6-N-acetylglucosaminyltransferase, β-1,3-galactosyltransferase and β-1,4-galactosyltransferase, described in more detail below.

Beta-1,3-N-acetyl-glucosaminyltransferase

A β-1,3-N-acetyl-glucosaminyltransferase is any protein which comprises the ability of transferring the N-acetyl-glucosamine of UDP-N-acetyl-glucosamine to lactose or another acceptor molecule, in a beta-1,3-linkage. Preferably, a β-1,3-N-acetyl-glucosaminyltransferase used herein does not originate in the species of the genetically engineered cell i.e., the gene encoding the β-1,3-galactosyltransferase is of heterologous origin. Non-limiting examples of β-1,3-N-acetyl-glucosaminyltransferase are given in table 6. β-1,3-N-acetyl-glucosaminyltransferase variants may also be useful, preferably such variants are at least 80%, such as at least 85%, such as at least 90, such as at least 95% identical to one of the β-1,3-N-acetyl-glucosaminyltransferase in table 6.

TABLE 6
List of β-1,3-N-acetyl-glucosaminyltransferase

| Protein Name | GenBank ID | origin |
|---|---|---|
| IgtA | WP_033911473.1 | Neisseria meningitidis |
| | WP_002248149 | |
| | AAF42258.1 | |
| | ELK60643.1 | |
| IgtA | AAK70338.1 | Neisseria gonorrhoeae |
| | ACF31229.1 | |
| IgtA | AAK02595.1 | Pasteurella multocida |
| IgtA | EEZ72046.1 | Neisseria cinerea |
| HD0466 | WP_010944479.1 | Haemophilus ducreyi |
| PmnagT | WP_014390683.1 | Pasteurella multocida |

In embodiments the glycosyltransferase encoded by the genetically engineered cell is an β-1,3-N-acetyl-glucosaminyltransferase from table 6. Preferably, the glycosyltransferase in the genetically engineered cell is a β-1,3-N-acetyl-glucosaminyltransferase from Neisseria meningitidis, such as the β-1,3-N-acetyl-glucosaminyltransferase of SEQ ID NO: 95 or a functional variant thereof.

### β-1,3-Galactosyltransferase

A β-1,3-Galactosyltransferase is any protein that comprises the ability of transferring the galactose of UDP-Galactose to a N-acetyl-glucosaminyl moiety to an acceptor molecule in a beta-1,3-linkage. Preferably, a β-1,3-galactosyltransferase used herein does not originate in the species of the genetically engineered cell i.e., the gene encoding the β-1,3-galactosyltransferase is of heterologous origin. Non-limiting examples of β-1,3-galactosyltransferases are given in table 7. β-1,3-galactosyltransferases variants may also be useful, preferably such variants are at least 80%, such as at least 85%, such as at least 90, such as at least 95% identical to one of the β-1,3-galactosyltransferases in table 7.

TABLE 7
List of beta-1,3-glycosyltransferases

| Protein | | |
|---|---|---|
| Name | GenBank ID | Origin |
| WbgO | WP_000582563.1 | E. coli 055:H7 |
| cpslBJ | AB050723.1 | Streptococcus agalactiae type lb Ol2 |
| Jhp0563 | AEZ55696.1 | Helicobacter pylori |
| GalTK | WP_111735921.1 | Helicobacter pylori ATCC 43504 |
| CvB3GalT | WP_080969100.1 | Chromobacterium violaceum |

In embodiments the at least one glycosyltransferase encoded by the genetically engineered cell is β-1,3-N-acetylglucosaminyltransferase and a β-1,3-galactosyltransferase. Preferably, the glycosyltransferase in the genetically engineered cell is the β-1,3-N-acetylglucosaminyltransferase is selected from table 4 and the β-1,3-galactosyltransferase is selected from table 5. Even more preferred the 1,3-N-acetylglucosaminyltransferase is from a Neisseria sp. and the β-1,3-galactosyltransferase is from Helicobacter pylori, such as the β-1,3-N-acetylglucosaminyltransferase with GenBank ref nr.

WP_002248149.1 and the β-1,3-galactosyltransferase with GenBank ref nr. WP_111735921.1 or SEQ ID NO: 96, or a functional variant thereof.

### β-1,4-Galactosyltransferase

A β-1,4-Galactosyltransferase is any protein that comprises the ability of transferring the galactose of UDP-Galactose to a N-acetyl-glucosaminyl moiety. Preferably, a β-1,4-galactosyltransferase used herein does not originate in the species of the genetically engineered cell i.e., the gene encoding the β-1,4-galactosyltransferase is of heterologous origin. Non-limiting examples of β-1,4-galactosyltransferases are given in table 8. β-1,4-galactosyltransferases variants may also be useful, preferably such variants are at least 80%, such as at least 85%, such as at least 90, such as at least 95% identical to one of the β-1,4-galactosyltransferases in table 8.

TABLE 8
List of beta-1,4-glycosyltransferases

| Protein Name | GenBank ID | Origin |
|---|---|---|
| GalT | WP_001262061.1 | Helicobacter pylori |
| LgtB | AAF42257.1 | Neisseria meningitidis MC58 |

In embodiments the at least one glycosyltransferase encoded by the genetically engineered cell is β-1,3-N-acetylglucosaminyltransferase and a β-1,4-galactosyltransferase. Preferably, the glycosyltransferase in the genetically engineered cell is the β-1,3-N-acetylglucosaminyltransferase is selected from table 4 and the β-1,4-galactosyltransferase is selected from table 6. Even more preferred the 1,3-N-acetylglucosaminyltransferase is from a Neisseria sp. and the β-1,4-galactosyltransferase is from Helicobacter pylori, such as the β-1,3-N-acetylglucosaminyltransferase with GenBank ref nr. WP_002248149.1 and the β-1,4-galactosyltransferase with GenBank ref nr. WP_001262061.1 or SEQ ID NO: 105, or a functional variant thereof.

### Alpha-1,2-Fucosyltransferase

An α-1,2-fucosyltransferase is a protein that comprises the ability to catalyze the transfer of fucose from a donor substrate, for example, GDP-fucose, to an acceptor molecule in an alpha-1,2-linkage. Preferably, an alpha-1,2-fucosyltransferase used herein does not originate in the species of the genetically engineered cell i.e., the gene encoding the alpha-1,2-fucosyltransferase is of heterologous origin. Non-limiting examples of alpha-1,2-fucosyltransferase are given in table 9. Alpha-1,2-fucosyltransferase variants may also be useful, preferably such variants are at least 80%, such as at least 85%, such as at least 90, such as at least 95% identical to one of the alpha-1,2-fucosyltransferase in table 9.

TABLE 9
List of α-1,2-fucosyltransferase

| Protein Name | GenBank ID | Origin |
|---|---|---|
| FutC | WP_080473865.1 | Helicobacter pylori ATCC 26695 |
| wbgL | ADN43847 | E. coli O126 |
| Smob | WP_126455392.1 | Sulfuriflexus mobilis |
| FucT2_HpUA802 | AAC99764.1 | Helicobacter pylori |
| FucT2_EcO126t | ABE98421.1 | Escherichia coli |
| FucT2_Hm12198 | CBG40460.1 | Helicobacter mustelae 12198 |
| FucT2_Pm9515 | ABM71599.1 | Brochlorococcus marinus str. MIT 9515 |
| FucT2_HpF57 | BAJ59215.1 | Helicobacter pylori F57 |
| FucT54 | ADE13114.1 | Sideroxydans lithotrophicus ES-11 |
| Mtun | WP_031437198.1 | Methylobacter tundripaludum |

In embodiments the glycosyltransferase encoded by the genetically engineered cell is an α-1,2-fucosyltransferase from table 7. Preferably, the glycosyltransferase in the α-1,2-fucosyltransferase is from Helicobacter pylori, such as the α-1,2-fucosyltransferase with the GenBank accession nr. WP_080473865.1 or SEQ ID NO: 108, or a functional variant thereof.

### Alpha-1,3-Fucosyltransferase

An alpha-1,3-fucosyltransferase refer to a glycosyltransferase that catalyzes the transfer of fucose from a donor substrate for example, GDP-fucose, to an acceptor molecule in an alpha-1,3-linkage. Preferably, an alpha-1,3-fucosyltransferase used herein does not originate in the species of the genetically engineered cell i.e., the gene encoding the alpha-1,3-fucosyltransferase is of heterologous origin. Non-limiting examples of alpha-1,3-fucosyltransferase are given in table 10. Alpha-1,3-fucosyltransferase variants may also be useful, preferably such variants are at least 80%, such as at least 85%, such as at least 90, such as at least 95% identical to one of the alpha-1,3-fucosyltransferase in table 10.

TABLE 10
List of α-1,3-fucosyltransferase

| Protein Name | GenBank ID | Origin |
|---|---|---|
| FucT | AAB81031.1 | Helicobacter pylori NCTC 11639 |
| FutA | WP_000487428.1 | Helicobacter pylori ATCC 26695 |
| CafC | WP_007483358.1 | Bacteroides nordii |
| FucT6_3_Bf | CAH09151.1 | Bacteroides fragilis NCTC 9343 |
| FucT7_3_Bf | CAH09495.1 | Bacteroides fragilis NCTC 9343 |
| FucT_3_Am | ACD04596.1 | Akkermansia muciniphila ATCC BAA-835 |
| MAMA_R764 | AGC02224. 1 | Acanthamoeba polyphaga moumouvirus |
| Mg791 | AEQ33441.1 | Megavirus chiliensis |
| Moumou_00703 | YP_007354660.1 | Acanthamoeba polyphaga moumouvirus |

In embodiments the glycosyltransferase encoded by the genetically engineered cell is an α-1,3-fucosyltransferase from table 8. Preferably, the glycosyltransferase in the genetically engineered cell is the α-1,3-fucosyltransferase FutA from Helicobacter pylori, such as the α-1,3-fucosyltransferase of SEQ ID NO: 89 or a functional variant thereof.

### Alpha-1,3/4-Fucosyltransferase

An alpha-1,3/4-fucosyltransferase refer to a glycosyltransferase that catalyzes the transfer of fucose from a donor substrate for example, GDP-fucose, to an acceptor molecule in an alpha-1,3- or alpha 1,4-linkage. Preferably, an alpha-1,3/4-fucosyltransferase used herein does not originate in the species of the genetically engineered cell i.e., the gene encoding the alpha-1,3/4-fucosyltransferase is of heterologous origin. Non-limiting examples of alpha-1,3/4-fucosyltransferase are given in table 11. alpha-1,3/4-fucosyltransferase variants may also be useful, preferably such variants are at least 80%, such as at least 85%, such as at least 90%, such as at least 95% identical to one of the alpha-1,3/4-fucosyltransferase in table 11.

TABLE 11
List of α-1,3/4-fucosyltransferase

| Protein | | |
|---|---|---|
| Name | GenBank ID | Origin |
| FucTIII | AY450598.1 | Helicobacter pylori strain DSM 6709 |
| FutA | WP_000487428.1 | Helicobacter pylori ATCC 26695 |

In embodiments the glycosyltransferase encoded by the genetically engineered cell is an α-1,3/4-fucosyltransferase from table 11. Preferably, the glycosyltransferase in the genetically engineered cell is the α-1,34-fucosyltransferase FutA from Helicobacter pylori, such as the α-1,34-fucosyltransferase of SEQ ID NO: 89 or a functional variant thereof.

### Alphα-2,3-Sialyltransferase

An α-2,3-sialyltransferase refer to a glycosyltransferase that catalyzes the transfer of sialyl from a donor substrate for example, CMP-N-acetylneuraminic acid, to an acceptor molecule in an alpha-2,3-linkage. Preferably, an alpha-2,3-sialyltransferase used herein does not originate in the species of the genetically engineered cell i.e., the gene encoding the 2,3-sialyltransferase is of heterologous origin. Non-limiting examples α-2,3-sialyltransferase are given in table 12. α-2,3-sialyltransferase variants may also be useful, preferably such variants are at least 80%, such as at least 85%, such as at least 90, such as at least 95% identical to one of the α-2,3-sialyltransferase in table 12.

TABLE 12
List of α-2,3-sialyltransferase

| Protein Name | GenBank ID | Origin |
|---|---|---|
| Avi | WP_115249238.1 | Avibacterium avium |
| Ccol | WP_075498955.1 | Campylobacter coli |
| Ccol2 | EAH6554614.1 | Campylobacter coli |
| Chepa | WP_066776435.1 | Campylobacter hepaticus |
| Cinf1 | WP_011272254.1 | Haemophilus influenzae |
| Cjej1 | EBD1936710.1 | Campylobacter jejuni |
| Clari1 | EGK8106227.1 | Campylobacter lari |
| CstI | AAF13495.1 | Campylobacter jejuni |
| CstII | AAF31771.1 | Campylobacter jejuni |
| Csub1 | WP_039664428.1 | Campylobacter subantarcticus |
| MhnNBse | WP_176810284.1 | Mannheimia (multispecies) |
| Neigon | AAW89748.1 | Neisseria gonorrhoeae FA 1090 |
| Nst | AAC44541.1 | Neisseria meningitidis MC58 |
| PM70 | AAK03258.1 | Pasteurella multocida subsp. |
| | | multocida str. Pm70 |
| PmN | WP_005726268.1 | Pasteurella (multispecies) |
| Pmult | WP_005753497.1 | Pasteurela multocida |
| Poral | WP_101774487.1 | Pasteurella oralis |
| Poral_2 | WP_101774701.1 | Pasteurella oralis |

In embodiments the glycosyltransferase encoded by the genetically engineered cell is an α-2,3-sialyltransferase from table 10. Preferably, the glycosyltransferase in the genetically engineered cell is a α-2,3-sialyltransferase from Campylobacter lari, Neisseria meningitidis or Pasteurella oralis, such as the α-2,3-sialyltransferase with the GenBank accession nr. EGK8106227.1, AAC44541.1, or WP_101774487.1 or SEQ ID NO: 91, or afunctional variant thereof

### Alpha-2,6-Sialyltransferase

An alpha-2,6-sialyltransferase refer to a glycosyltransferase that catalyzes the transfer of sialyl from a donor substrate for example, CMP-N-acetylneuraminic acid, to an acceptor molecule in an alpha-2,6-linkage. Preferably, an alpha-2,6-sialyltransferase used herein does not originate in the species of the genetically engineered cell i.e., the gene encoding the 2,6-sialyltransferase is of heterologous origin. Non-limiting examples α-2,6-sialyltransferase are given in table 13. α-2,6-sialyltransferase variants may also be useful, preferably such variants are at least 80%, such as at least 85%, such as at least 90, such as at least 95% identical to one of the α-2,6-sialyltransferase in table 13.

TABLE 13
List of α-2,6-sialyltransferase

| Protein | | |
|---|---|---|
| Name | GenBank ID | Origin |
| HAC1268 | STP03859.1 | Helicobacter acinonychis str. Sheeba |
| Pts6-119 | AB500947. 1 | Photobacterium leiognathi JT-SHIZ-119 |
| Pts6-224 | BAF92026.1 | Photobacterium sp JT-ISH-224 |
| PiST6_145 | BAF91416.1 | Photobacterium leiognathi |

In embodiments the glycosyltransferase encoded by the genetically engineered cell is α-2,6-sialyltransferase from table 11. the glycosyltransferase in the genetically engineered cell is a α-2,6-sialyltransferase from Photobacterium sp, such as the α-2,6-sialyltransferase with the GenBank accession nr. AB500947.1 or BAF92026.1.

### Nucleotide-Activated Sugar Pathways

In the genetically engineered cells used in the methods described herein, a glycosyltransferase mediated glycosylation reaction takes place inside the cell, in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably the following activated sugar nucleotides are involved in the glycosyl transfer: glucose-UDP-GlcNAc, UDP-galactose, UDP-glucose, UDP-N-acetylglucosamine, UDP-N-acetylgalactosamine (GlcNAc) and CMP-N-acetylneuraminic acid. The genetically modified cells according to the present invention can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid (CMP-Neu5Ac).

In embodiments, the genetically modified cells are capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, maltose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).

The enzymes involved in the de novo biosynthetic pathway of an activated sugar nucleotide can be naturally present in the cells or introduced into the cells by means of gene technology or recombinant DNA techniques, all of them are part of the general knowledge of the skilled person.

In embodiments the pathway to produce a nucleotide-activated sugar is the de novo GDP-fucose pathway (gmd, wcaG, manB, manC and manA) and/or the sialic acid sugar nucleotide pathway (neuB, neuC and neuA) as described below.

In another embodiment, the genetically modified cells can utilize salvaged monosaccharides for sugar nucleotide synthesis. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cells.

### Colanic Acid Gene Cluster

For the production of fucosylated oligosaccharides/HMOs the colanic acid gene cluster is important to ensure presence of sufficient GDP-fucose. In Escherichia coli GDP-fucose is an intermediate in the production of the extracellular polysaccharide colanic acid, a major oligosaccharide of the bacterial cell wall. In the context of the present invention the colanic acid gene cluster (from E. coli it is shown as SEQ ID NO: 109) encodes most of the enzymes involved in the de novo synthesis of GDP-fucose (gmd, wcaG, wcaH, wcaI, manB, manC), whereas one or several of the genes downstream of GDP-L-fucose, such as wcaJ, can be deleted to prevent conversion of GDP-fucose to colanic acid.

The de novo GDP-fucose pathway genes responsible for the formation of GDP-fucose comprises or consists of the following genes: i) manA which encodes the protein mannose-6 phosphate isomerase (EC 5.3.1.8, UniProt accession nr. P00946), which facilitates the interconversion of maltose 6-phosphate (F6P) and mannose-6-phosphate; ii) manB which encodes the protein phosphomannomutase (EC 5.4.2.8, UniProt accession nr P24175), which is involved in the biosynthesis of GDP-mannose by catalyzing conversion mannose-6-phosphate into mannose-1-phosphate; iii) manC which encodes the protein mannose-1-phosphate guanylyltransferase guanylyltransferase (EC:2.7.7.13, UniProt accession nr P24174), which is involved in the biosynthesis of GDP-mannose through synthesis of GDP-mannose from GTP and α-D-mannose-1-phosphate; iv) gmd which encodes the protein GDP-mannose-4,6-dehydratase (UniProt accession nr P0AC88), which catalyzes the conversion of GDP-mannose to GDP-4-dehydro-6-deoxy-D-mannose; v) wcaG (fcl) which encodes the protein GDP-L-fucose synthase (EC 1.1.1.271, UniProt accession nr P32055) which catalyses the two-step NADP-dependent conversion of GDP-4-dehydro-6-deoxy-D-mannose to GDP-fucose.

Accordingly, it is preferred that the genetically engineered cell, when producing one or more fucosylated heterologous products, overexpresses either the entire colonic acid gene cluster and/or one or more genes of the de novo GDP-fucose pathway selected from the group consisting of manA, manB, manC, gmd and wcaG.

In one or more exemplary embodiment(s), the colanic acid gene cluster responsible for the formation of GDP-fucose may be expressed from its native genomic locus. The expression may be actively modulated to increase GDP-fucose formation. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

In relation to the present disclosure, the term “native genomic locus”, in relation to the colanic acid gene cluster, relates to the original and natural position of the gene cluster in the genome of the genetically engineered cell.

### Sialic Acid Sugar Nucleotide Synthesis Pathway

If the genetically modified cell is to produce a sialylated oligosaccharide/HMO the genetically modified cell comprises a sialic acid sugar nucleotide synthesis capability, i.e., the genetically modified cell comprises a biosynthetic pathway for making a sialate sugar nucleotide, such as CMP-N-acetylneuraminic acid as glycosyl-donor for the sialyltransferases. E.g., the genetically modified cell comprises a sialic acid synthetic capability through provision of an exogenous UDP-GlcNAc 2-epimerase (e.g., neuC of Campylobacter jejuni (GenBank AAK91727.1) or equivalent (e.g., (GenBank CAR04561.1), a Neu5Ac synthase (e.g., neuB of C. jejuni (GenBank AAK91726.1) or equivalent, (e.g., Flavobacterium limnosediminis sialic acid synthase, GenBank WP_023580510.1), and/or a CMP-Neu5Ac synthetase (e.g., neuA of C. jejuni (GenBank AAK91728.1) or equivalent, (e.g., Vibrio brasiliensis CMP-sialic acid synthase, GenBank WP_006881452.1). Disclosed as SEQ ID NO: 94 herein is an example of a neuBCA gene cluster from Campylobacter jejuni, alternative functional variants are also suitable for making a sialate sugar nucleotide in a genetically modified cell.

Furthermore, the genetically modified cell preferably has a deficient sialic acid catabolic pathway. By “sialic acid catabolic pathway” is meant a sequence of reactions, usually controlled, and catalysed by enzymes, which results in the degradation of sialic acid. An exemplary sialic acid catabolic pathway described hereafter is the E. coli pathway. In this pathway, sialic acid (Neu5Ac; N-acetylneuraminic acid) is degraded by the enzymes NanA (N-acetylneuraminic acid lyase) and NanK (N-acetylmannosamine kinase) and NanE (N-acetylmannosamine-6-phosphate epimerase), all encoded from the nanATEK-yhcH operon, and repressed by NanR (http://ecocyc.org/ECOLI). An inactivated sialic acid catabolic pathway is rendered in the E. coli host by introducing one or more mutations in the endogenous nanA (N-acetylneuraminate lyase) (e.g., GenBank Accession Number D00067.1(GL216588)) and/or nanK (N-acetylmannosamine kinase) genes (e.g., GenBank Accession Number (amino acid) BAE77265.1 (GL85676015)), and/or nanE (N-acetylmannosamine-6-phosphate epimerase, GI: 947745), incorporated herein by reference). Optionally, the nanT (N-acetylneuraminate transporter) gene is also inactivated or mutated. By inactivated is meant that the coding sequence has been altered such that the resulting gene product is functionally inactive or encodes for a gene product with less than 100%, e.g., 90%, 80%, 70%, 60%, 50%, 40%, 30% or 20% of the activity of the native, naturally occurring, endogenous gene product. Thus, in the present invention, nanA, nanK, nanE, and/or nanT genes are preferably inactivated.

### Lactose Importer

In embodiments where the genetically modified cells use lactose, or alternatively LacNAc, LNB, 2′FL or 3FL as the initial substrate for the oligosaccharide formation it is preferable that the cells are capable of importing the substrate into the cell, unless the initial substrate is produced in-situ by the cell itself.

In embodiments of the two-strain hybrid method at least one of the genetically modified cells are capable of importing the side-product produced as the leaving group in the transglycosylation process (enzymatic process) of the hybrid process into the cell.

In embodiments of the two-strain hybrid method the genetically modified cell comprises a side-product importer. Preferably the side-product importer can import one or more of the following side-products lactose, 2′FL and/or 3FL.

Most lactose importers are capable of importing both lactose, LNB, LacNAc and 2′FL. In embodiments where the initial substrate for the oligosaccharide production or the side-product from the two-strain hybrid process is lactose, LNB, LAcNAc or 2′FL the genetically modified cell has a functional lactose importer or a 2′FL importer. Lactose importers are well known in a wide variety of species including bacteria and yeasts.

The lactose importer can for example be a lactose permease. The lactose permease may be an endogenous lactose permease natively expressed by the cell used to produce the first oligosaccharide.

In one or more embodiment(s), one or both genetically modified cells comprise one or more lactose permease genes which is/are overexpressed.

One or both genetically engineered cells may comprise least one, such as at least two, three, four, nucleic acid sequence(s) encoding a lactose permease.

In one or more further exemplary embodiment(s) the one or more lactose permease(s) is/are encoded by a heterologous and/or recombinant nucleic acid sequence. The native lactose permease may be genetically engineered to for example place it under control of a stronger promoter than the native promoter, thereby generating a recombinant lactose permease gene overexpressing the native lactose permease protein.

In one or more preferred exemplary embodiment(s) the nucleic acid sequence(s) encoding the one or more lactose permease(s) is a native gene of the genetically engineered cell.

In E. coli the lactose permease is encoded by the lacY gene in the lac operon. In exemplary embodiments, the lactose permease in the genetically modified cell is LacY from E. coli. Preferably the Lactose permease comprises or consists of an amino acid sequence of SEQ ID NO: 1 or a functional homologue thereof, such as a lactose permease having an amino acid sequence which is at least 80%, such as at least 90%, such as at least 95%, such as at least 99%, or 100% identical to SEQ ID NO: 1.

### Transporter Protein for Oligosaccharide Export

As described above it is preferable that the first and second genetically modified cells grown in the co-culture are capable of exporting the first and second oligosaccharide produce, respectively. This is in particularly preferred for the two-strain hybrid process where the first and second oligosaccharides are subjected to an enzymatic reaction in the culture broth. Likewise in the two-strain system where the first oligosaccharide is an intermediate oligosaccharide imported by the second genetically modified cell it is desired that the oligosaccharide produced by the first genetically modified cell is exported to the culture medium to make it available to the second cell.

In embodiments of the present disclosure the genetically modified cell(s) preferably comprises at least one nucleic acid sequence encoding one or more transporter protein(s) capable of exporting the first and/or second oligosaccharide from the cell into the culture medium. The genetically modified cell of the present disclosure preferably expresses a Sugar Efflux Transporter (SET) transporter protein or a heterologous Major Facilitator Superfamily (MFS) transporter protein.

Sugar Efflux Transporter (SET) transporters capable of exporting certain HMOs are described in WO2010142305. In particular SetA with UniProt accession nr P31675 or functional variants thereof may be useful in the export of 2′FL and 3FL.

The transporters of the Major Facilitator Superfamily (MFS) facilitate the transport of molecules, such as but not limited to oligosaccharides, across the cellular membranes.

The term “MFS transporter” in the present context means a protein that facilitates transport of an oligosaccharide, preferably an HMO, through or across a cell membrane, from the cell cytosol to the cell periplasm and/or medium. Preferably, the MFS transporter transports an HMO/oligosaccharide synthesized by the genetically modified cell as described herein. Additionally, or alternatively, the MFS transporter may also facilitate efflux of molecules that are not considered HMO or oligosaccharides, such as lactose, glucose, cell metabolites and/or toxins. In a preferred embodiment the MFS transporter is capable of exporting 2′FL, 3FL, 3′SL, 6′SL, LNT-II, LNT, LNnT and/or LNFP-I from the cell cytosol to the cell medium.

In the context of the present invention the lactose permease is not considered to be a heterologous MFS transporter.

In one or more exemplary embodiment(s), the MFS transporter is selected from the group consisting of Bad, Nec, YjhB, YberC, Fred, Vag and Marc.

The genetically modified cell of the present disclosure thus in one or more exemplary embodiment(s) expresses a heterologous MFS transporter protein selected from the group consisting of Vag, Nec, Fred, Marc, YberC, Bad and a functional homologue of any one of Vag, Nec, Fred, Marc, YberC or Bad having an amino acid sequence which is 80% identical to said.

### Bad

The MFS transporter protein identified herein as “Bad protein” or “Bad transporter” or “Bad”, has an amino acid sequence corresponding to the GenBank accession ID WP_017489914.1.

In one or more embodiment(s) of the invention, the genetically engineered cell expresses the heterologous MFS transporter protein bad or a functional homologue thereof, having an amino acid sequence which is at least 80%, such as at least 90%, such as at least 95%, such as at least 99%, or 100% identical to the GenBank accession ID WP_017489914.1

### Nec

The MFS transporter protein identified herein as “Nec protein” or “Nec transporter” or “Nec”, interchangeably, has an amino acid sequence corresponding to SEQ ID NO: 107 or the GenBank accession ID WP_092672081.1.

In one or more embodiment(s) of the invention, the genetically engineered cell expresses the heterologous MFS transporter protein Nec or a functional homologue thereof, having an amino acid sequence which is at least 80%, such as at least 90%, such as at least 95%, such as at least 99%, such as 100% identical to the amino acid sequence of SEQ ID NO: 107 or the GenBank accession ID WP_092672081.1.

Nec is in particularly suitable for transporting 2′FL, DFL, LNT, 3′SL, 6′SL and LNFP-I.

### YjhB

The MFS transporter protein identified herein as “YhjB protein” or “YjhB transporter” or “YjhB”, interchangeably, has an amino acid sequence corresponding UniProt accession ID P39352.

In one or more embodiment(s) of the invention, the genetically engineered cell expresses the heterologous MFS transporter protein YjhB or a functional homologue thereof, having an amino acid sequence which is at least 80%, such as at least 90%, such as at least 95%, such as at least 99%, such as 100% identical to the amino acid sequence UniProt accession ID P39352.

YjhB is particularly useful in exporting LNT-II.

### YberC

The MFS transporter protein identified herein as “YberC protein” or “YberC transporter” or “YberC”, interchangeably, has an amino acid sequence corresponding to SEQ ID NO: 103 or the GenBank accession ID EEQ08298.1.

In one or more embodiment(s) of the invention, the genetically engineered cell expresses the heterologous MFS transporter protein YberC or a functional homologue thereof, having an amino acid sequence which is at least 80%, such as at least 90%, such as at least 95%, such as at least 99%, such as 100% identical to the amino acid sequence of SEQ ID NO: 103 or the GenBank accession ID EEQ08298.1.

YberC is particularly useful in transporting LNT.

### Fred

The MFS transporter protein identified herein as “Fred protein” or “Fred transporter” or “Fred”, interchangeably, has an amino acid sequence corresponding the GenBank accession ID WP_087817556.1.

In one or more exemplary embodiment(s), the MFS transporter, expressed according to the present disclosure is Fred. The genetically engineered cell of the present disclosure thus in one or more exemplary embodiment(s) expresses a heterologous MFS transporter protein that is Fred.

In one or more embodiment(s) of the invention, the genetically engineered cell expresses the heterologous MFS transporter protein fred or a functional homologue thereof, having an amino acid sequence which is at least 80%, such as at least 90%, such as at least 95%, such as at least 99%, such as 100% identical to the amino acid sequence of the GenBank accession ID WP_087817556.1.

Fred is particularly useful in transporting 3′SL and 6′SL.

### Vag

The MFS transporter protein identified herein as “Vag protein” or “Vag transporter” or “Vag”, interchangeably, has an amino acid sequence corresponding SEQ ID NO: 106 or the GenBank accession ID WP_048785139.1.

In one or more embodiment(s) of the invention, the genetically engineered cell expresses the heterologous MFS transporter protein vag or a functional homologue thereof, having an amino acid sequence which is at least 80%, such as at least 90%, such as at least 95%, such as at least 99%, such as 100% identical to the amino acid sequence of SEQ ID NO: 106 or the GenBank accession ID WP_048785139.1.

Vag is particularly useful in transporting LNnT.

### Marc

The MFS transporter protein identified herein as “Marc protein” or “Marc transporter” or “Marc”, interchangeably, has an amino acid sequence corresponding SEQ ID NO: 90 or the GenBank accession WP_060448169.1.

In one or more embodiment(s) of the invention, the genetically engineered cell expresses the heterologous MFS transporter protein marc or a functional homologue thereof, having an amino acid sequence which is at least 80%, such as at least 90%, such as at least 95%, such as at least 99%, such as 100% identical to the amino acid sequence of SEQ ID NO: 90 or the GenBank accession WP_060448169.1.

Marc is particularly useful in transporting 3FL.

### Edic1

The MFS transporter protein identified herein as “Edic1 protein” or “Edic1 transporter” or “Edic1” interchangeably from Edwardsiella ictalurid identified has an amino acid sequence corresponding SEQ ID NO: 60 or GenBank accession WP_015873007.1.

In one or more embodiment(s) of the invention, the genetically engineered cell expresses the heterologous MFS transporter protein marc or a functional homologue thereof, having an amino acid sequence which is at least 80%, such as at least 90%, such as at least 95%, such as at least 99%, such as 100% identical to the amino acid sequence of SEQ ID NO: 60 or GenBank accession WP_015873007.1

Edic1 is particularly useful for transporting LNT or LNnT.

The genetically engineered cell of the present disclosure thus in one or more exemplary embodiment(s) expresses a heterologous MFS transporter protein which is either Vag, Nec, Fred, Marc, Edic1, YjhB, YberC or Bad.

In one or more exemplary embodiment(s), the genetically engineered cell of the present disclosure expresses a functional homologue of Vag, Nec, Fred, Marc, Edic1, YjhB, YberC and/or Bad having an amino acid sequence which is at least 70%, 80%, 85%, 90%, 95% or at least 99% identical to the Vag, Nec, Fred, Marc, edic 1, YberC and/or Bad GenBank ascension numbers indicated above.

In a presently preferred embodiment, the MFS transporter expressed is Nec.

In an especially preferred embodiment, the MFS transporter expressed is YberC.

In an especially preferred embodiment, the MFS transporter expressed is Marc.

In an especially preferred embodiment, the MFS transporter expressed is Edic1.

In an especially preferred embodiment, the MFS transporter expressed is YjhB,

In an especially preferred embodiment, the MFS transporter expressed is Vag.

In an especially preferred embodiment, the MFS transporter expressed is Fred

### Sequence Identity

The term “sequence identity” as used herein describes the relatedness between two amino acid sequences or between two nucleotide sequences, i.e., a candidate sequence (e.g., a sequence of the invention) and a reference sequence (such as a prior art sequence) based on their pairwise alignment.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “identity” (obtained using the -nobrief option) is used as the percent identity. Generally sequence identity may be calculated as follows: (Identical Residues×100)/(Length of Aligned region).

For purposes of the present invention, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “identity” (obtained using the -nobrief option) is used as the percent identity. Generally sequence identity may be calculated as follows: (Identical Deoxyribonucleotides×100)/(Length of Aligned region).

Functional homologue A functional homologue or functional variant of a protein/nucleic acid sequence as described herein is a protein/nucleic acid sequence with alterations in the genetic code or amino acid sequence, which retains its original functionality. A functional variant may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence. In embodiments of the present invention, the functional variant is at least 80% identical, such as at least 85% identical such as at least 90% identical, such as such as at least 95% identical to the protein/nucleic acid sequence indicated in connection with a give protein, nucleic acid or gene.

Functional variants or homologues may also be across species, i.e., different species such as, but not limited to, E. coli, Bacillus, Corynebacterium, Lactobacillus, Saccharomyces may have proteins with similar function such as various sugar transport systems including phosphoenolpyruvate:sugar phosphotransferase systems (PTS). In the context of the present disclosure such systems which are present across multiple species are considered functional variants. For ease only one NCBI or UniProt reference has been provided to characterize such proteins, however it is understood that proteins with equivalent function from other species are to be considered as functional variants.

Functional variants of proteins or peptides may contain conservative amino acid substitution(s) compared to their native, i.e., non-mutated physiological, sequence. Those amino acid sequences as well as their encoding nucleotide sequences in particular fall under the term functional variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g., side chains which have a hydroxyl function. This means that e.g., an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Truncations, insertions and substitutions are possible, in particular, at those sequence positions which cause no modification to the three-dimensional structure or do not affect the binding region. Modifications to a three-dimensional structure by insertion(s) or deletion(s) can easily be determined e.g., using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

Furthermore, functional variants of proteins or peptides as defined herein may also comprise those sequences, wherein nucleotides of the nucleic acid are exchanged according to the degeneration of the genetic code, without leading to an alteration of the respective amino acid sequence of the protein or peptide, i.e., the amino acid sequence or at least part thereof may not differ from the original sequence in one or more mutation(s) within the above meaning.

### Retrieving/Harvesting

The one or more oligosaccharides produced using the two-strain system (co-culturing) method described herein can be retrieved from the culture medium and/or the cells of the process. In the present context, the term “retrieving” is used interchangeably with the term “harvesting”. Both “retrieving” and “harvesting” in the context relate to collecting the produced HMO(s) from the culture (cells)/broth (culture medium) following the termination of the process. In the two-strain hybrid process the biomass (cells) is preferably discarded since these only contain the first and second oligosaccharide, and the oligosaccharide of interest is the complex fucosylated or sialylated oligosaccharide produced by the enzymatic reaction in the culture medium.

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions.

After recovery of the hybrid process medium, the HMO mixture/composition is available for further processing and purification. It may be desirable to isolate individual HMOs from the HMO mixture to obtain e.g., a purified or enriched sialylated and/or a fucosylated oligosaccharide of at least four monosaccharide. Alternatively, the HMO mixture produced by the hybrid process can be purified to remove lactose and other metabolic non-HMO byproducts (e.g., by ultra-filtration and/or nanofiltration) and the HMO mixture or HMO composition can be used as it is.

The purification of the HMO composition or a specific component of the HMO mixture can be done according to procedures known by the skilled artesian. For example, HMOs can be purified according to the procedures known in the art, e.g., such as described in in WO2015/188834, WO2017/182965 or WO2017/152918, wherein the latter describes purification of HMOs.

### Mixtures of Oligosaccharides, Such as HMOs, and Compositions Thereof

The methods described herein produce one or more oligosaccharides. Often the process produces a mixture of oligosaccharides. With the co-culturing approach, a mixture of two HMOs in a desired ratio, such a mixture may also contain smaller amounts, such as less than 20%, such as less than 15%, such as less than 10% of by-product oligosaccharides (e.g. intermediates such as LNT-II in the production of LNT and LNnT, or 2′FL and 3FL in the production of DFL). After the fermentation the by-product oligosaccharides may also be higher, but these can be reduced to the desired levels in a down-stream purification process generating the desired composition of oligosaccharides such as HMOs.

Complex fucosylated and/or sialylated HMOs, such as LST-c and LST-a mixtures are highly relevant as either a nutritional supplement or as a therapeutic as described in the section of uses of HMO mixtures described in the present application.

An aspect of the present disclosure is a mixture or composition of HMOs consisting essentially of:
a) at least 40 wt % LST-c, below 25 wt % LNnT, below 25 wt % 6′SL and below 10 wt % lactose, or
b) at least 60 wt % LST-a, below 30 wt % LNT, below 15 wt % 3′SL and below 2 wt % lactose, and
   wherein the total composition constitutes 100 wt % of the components and the composition is a mixture of at least 2 components.

In embodiments the composition or mixture of HMOs consists essentially of
a) at least 50 wt % LST-c, between 15 to 25 wt % LNnT, between 15 to 25 wt % 6′SL and between 0 to 7 wt % lactose,
b) at least 60 wt % LST-a, between 15 to 30 wt % LNT, between 0 to 15 wt % 3′SL and between 0 to 2 wt % lactose and
   wherein the total composition constitutes 100 wt % of the components.

In other embodiments, the mixture of HMOs described herein consists essentially of 45-55 wt % LST-c, and 20-30 wt % LNnT and 20-30 wt % 6′SL, wherein the total composition constitutes 100 wt % of the components.

In other embodiments, the mixture of HMOs described herein consists essentially of 50 wt % LST-c, and 25 wt % LNnT and 25 wt % 6′SL.

In other embodiments, the mixture of HMOs described herein consists essentially of 60 to 80 wt % LST-a and 20 to 30 wt % LNT and 5 to 15 wt % 3′SL, wherein the total composition constitutes 100 wt % of the components.

In other embodiments, the mixture of HMOs described herein consists essentially of 65 wt % LST-a and 25 wt % LNT and 10 wt % 3′SL.

As shown in the examples, the hybrid method of the present disclosure allows for improved ratios of the desired complex fucosylated or sialylated HMO over the donor and/or acceptor HMO as compared to the conventional in vitro process where the ratios between the individual HMOs is limited by the kinetic barrier of the enzymatic reaction, preventing shifts of the equilibrium between the components of the in vitro enzymatic reaction.

In other embodiments the composition and/or mixture of HMOs has a molar ratio of LST-c:LNnT above 2.5:1 and the molar ratio of LST-c:6′SL above 2.5:1.

In other embodiments the composition and/or mixture of HMOs has a molar ratio of LST-a:LNT above 1.5:1 and the molar ratio of LST-a:3′SL above 8:1.

In embodiments, the composition comprising a mixture of HMOs is a nutritional composition. Nutritional compositions are for example infant formula or medical nutritional compositions.

In embodiments, the composition comprising a mixture of HMOs is dietary supplement.

In embodiments, the composition comprising a mixture of HMOs is a pharmaceutical composition.

### Use of HMO Mixtures and Compositions

Clinical data in infants, indicate that human milk oligosaccharide supplements may help to develop the desired microbiota by serving as a food source for the good bacteria in the intestine. Naturally occurring in breast milk, HMOs have evolved over thousands of years, with HMO research (clinical and preclinical) now suggesting that specific HMO's at the correct level of supplementation can provide us with unique health benefits. In particular, Human Milk Oligosaccharide supplements may help support immunity and gut health including a support a balanced microbiome, with a potential role in cognitive development, which may open future innovation opportunities.

Accordingly, in embodiments, the invention relates to the use of a mixture or composition disclosed herein in infant nutrition.

The present invention also relates to the use of a mixture or composition disclosed herein as a dietary supplement or medical nutrition or a pharmaceutical composition.

The mixtures or composition of HMOs produced according to the method described herein may be used to enhance the beneficial bacteria in the gut microbiome. Beneficial bacteria are for example bacteria of the Bifidobacterium sp., Lactobacillus sp. or Barnesiella sp. The enhancement of beneficial bacteria may in turn lead to increased production of short chain fatty acids (SCFAs) such as acetate, propionate and butyrate which have been shown to have many benefits in infants and young children, such as inhibition of pathogen bacteria, prevention of infection and diarrhoea, reduced risk of allergy and metabolic disorders (see for example WO2006/130205, WO 2017/129644, WO2017/129649).

The mixtures or composition of HMOs produced according to the method described herein may be used to reduce the abundance of undesirable viruses and bacteria in the gut microbiome. Examples of pathogenic bacteria and viruses that may be reduced by the HMO mixtures described herein are including Candida albicans, Clostridium difficile, Enterococcus faecium, Escherichia coli, Helicobacter pylori, Streptococcus agalactiae, Shigella dysenteriae, Staphylococcus aureus, nora virus and rota virus. Each composition described herein can also be used to treat and/or reduce the risk of a broad range of bacterial infections of a human.

The mixtures or composition of HMOs produced according to the method described herein may be used to increase the regeneration and viability of lyophilized probiotics, including probiotics of Bifidobacterium sp, and Lactobacillus sp., in particular increased regeneration and/or viability and/or shelf-life in an acidic environment, such as the stomach or acidic food products, is an advantage using the HMO mixtures described herein. Examples of Bifidobacterium sp which may have increased regeneration and viability are Bifidobacterium animals lactis BB12 DSM 32269, Bifidobacterium animals lactis BIF6, Bifidobacterium longum DSM 32946, Bifidobacterium longum BB536, Bifidobacterium bifidum DSMZ 32403, Bifidobacterium infantis, Bifidobacterium breve DSM 33789, Bifidobacterium infantis SP37 DSM 32687, Bifidobacterium adolescentis DSM 34065 and/or Bifidobacterium animalis ssp. animalis DSM 16284. Examples of Lactobacillus sp which may have increased regeneration and viability are Lactobacillus rhamnosus GG DSM 32550, Lactobacillus rhamnosus 19070-2 DSM 26357, Lactobacillus rhamnosus GG, Lactobacillus rhamnosus LBrGG, Lactobacillus reuteri DSM 12246, Lactobacillus plantarum TIFN101, Lactobacillus gasseri Lg-36 200B FloraFit Danisco, Lactobacillus casei DSM 32382, Lactobacillus paracasei, Lactobacillus plantarum PS 128, Lactobacillus plantarum (Sacco) DSM 32383, Lactococcus lactis PAREVE, Lactobacillus paracasei ssp. Paracasei and/or Lactobacillus Probio-Tec®LGG®, Limosilactobacillus reuteri S12 DSM 33752.

In the context of the present application “Regeneration” means the process of regaining/restoring a dried bacteria's viability (i.e., “reviving” the bacterial cells by rehydration, wherein “rehydration” means restoring fluid). This process is also sometimes referred to as “reconstitution”.

In the context of the present application “Viability” is the ability of a bacterial cell to live and function as a living cell. One way of determining the viability of bacterial cells is by spreading them on an agar plate with suitable growth medium and counting the number of colonies formed after incubation for a predefined time (plate counting). Alternatively, FACS analysis may be used.

In the context of the present application “Improving the regeneration” of Bifidobacterium sp and/or Lactobacillus sp bacteria means to increase the amount (number) of Bifidobacterium sp and/or Lactobacillus sp. bacteria successfully regenerating/reviving compared to the respective control (i.e., the amount/number of Bifidobacterium sp and/or Lactobacillus sp. bacteria without the addition of HMO).

In the context of the present application “Improving the viability” of Bifidobacterium sp and/or Lactobacillus sp bacteria means to increase the amount (number) of viable Bifidobacterium sp and/or Lactobacillus sp. bacteria compared to the respective control (i.e., the amount/number of Bifidobacterium sp and/or Lactobacillus sp. bacteria without the addition of HMO).

In the context of the present application “acidic” means having a pH below 7.0 (for example, having a pH ≤6.0, or 5.0, or ≤4.0, or ≤3.0, or in the range of 1.0-6.0, such as from 2.0 to 5.0). The pH measured in the stomach is in the range of about 1.5-3.5. The pH measured in a healthy vagina is in the range of about 3.8-5.0. The pH of fruit juices is in the range of about 2.0-4.5.

The mixtures or composition of HMOs produced according to the method described herein, may be used to extend the shelf life of probiotics, such as Bifidobacterium sp, and/or Lactobacillus sp.

An embodiment of the present invention is a composition comprising a mixture of HMOs as described herein, in particular in the section “Mixtures of HMOs”, and one or more probiotics. Preferably, the probiotic is a Bifidobacterium sp and/or Lactobacillus sp such as any of the specific species mentioned above.

The mixtures or composition of HMOs produced according to the method described herein, may be used to improve the flowability of a powder or decrease the viscosity of a liquid.

The mixtures or composition of HMOs produced according to the method described herein are used in a nutritional composition. Nutritional compositions are for example, an infant formula, a rehydration solution, or a dietary maintenance, medical nutrition or supplement for elderly individuals or immunocompromised individuals. Macronutrients such as edible fats, carbohydrates and proteins can also be included in such anti-infective compositions. Edible fats include, for example, coconut oil, soy oil and monoglycerides and diglycerides. Carbohydrates include, for example, glucose, edible lactose and hydrolysed cornstarch.

Proteins include, for example, soy protein, whey, and skim milk. Vitamins and minerals (e. g. calcium, phosphorus, potassium, sodium, chloride, magnesium, manganese, iron, copper, zinc, selenium, iodine, and Vitamins A, E, D, C, and B complex) can also be included in such anti-infective compositions.

### Sequence List

The current application contains a sequence listing in text format and electronical format which is hereby incorporated by reference.

An overview of the SEQ ID NOs used in the present application are shown in the table below.

Summary of sequences listed in the application:

| Name | Comments | SEQ ID NO |
|---|---|---|
| lacY | Lactose permease LacY, GenBank ID: NP_414877.1, | 1 |
| Oligosaccharide | Listed in table 2 | 2 to 50 |
| importers | | |
| Transsialidases | Table 4 | 51-61, 87, |
| | | 88 and 113 |
| Transfucosidases | Table 5 | 62-86, 123 |
| FutA | ncbi sequence ID: NP_207177.1, alpha-1,3- | 89 |
| | fucosyltransferase from Helicobacter pylori 26695 | |
| marc | ncbi sequence ID: WP_060448169.1, MFS transporter | 90 |
| | protein from Serratia marcescens capable of exporting 3FL | |
| D29nst | α-2,3-sialyltransferase | 91 |
| Plac promoter | Wildtype promoter from E. coli | 92 |
| PglpF promter | Promoter sequence | 93 |
| neuBCA | Nucleic acid sequence encoding the neuBCA cluster from | 94 |
| | Campylobacter jejuni | |
| IgtA | β-1,3-N-acetyl-glucosaminyltransferase | 95 |
| galTK | β-1,3-Galactosyltransferase | 96 |
| ScrY | ncbi sequence ID: CAA40657.1, ScrY porin protein from | 97 |
| | Klebsiella pneumoniae | |
| ScrA | ncbi sequence ID: CAA40658.1, Sucrose transport protein | 98 |
| | Enzyme II scr protein from Klebsiella pneumoniae | |
| ScrB | ncbi sequence ID: WP_000056853.1, ScrB invertase | 99 |
| | enzyme from Salmonella enterica subsp. enterica serovar | |
| | Typhimurium | |
| ScrR | ncbi sequence ID: WP_000851062.1, Scr Repressor protein | 100 |
| | from Salmonella enterica subsp. enterica serovar | |
| | Typhimurium | |
| PglF-SD1 promoter | Variant of the PglpF promoter sequence | 101 |
| Pscr promoter | Wildtype promoter of the from Klebsiella pneumoniae | 102 |
| yberC | MFS transporter protein from Yersinia bercovieri capable of | 103 |
| | exporting LNT | |
| ST6, Pd2 | α-2,6-sialyltransferase | 104 |
| galT | β-1,4-Galactosyltransferase | 105 |
| vag | GenBank accession ID WP_048785139.1, MFS transport | 106 |
| | protein capable of exporting LNnT | |
| nec | GenBank accession ID WP_WP_092672081.1, MFS | 107 |
| | transport protein capable of exporting at least 2′FL, DFL, | |
| | LNT, 3′SL, 6′SL and LNFP-I. | |
| futC | alpha-1,2-fucosyl-transferase from Helicobacter pylori | 108 |
| CA | colanic acid gene cluster (gmd, wcaG, wcaH, wcal, manB, | 109 |
| | manC) | |
| cscABKR-gene | PTS dependent sucrose system from E. coli | 110 |
| cluster | | |
| SacC_Agal | Sucrose invertase | 111 |
| Bff | Sucrose invertase | 112 |
| Signal sequences | Signal sequences | 114-121 and |
| | | 127-146 |
| LNbX | trans-lacto-N-biosidase | 122 |
| Edic1 | GenBank accession ID WP_015873007.1, MFS transport | 124 |
| | protein capable of exporting LNT and LNnT | |
| Poral | α-2,3-sialyltransferase | 125 |
| PmglB_70UTR_SD4 | Promoter sequence | 126 |

### EMBODIMENTS

The following embodiments of the present invention may be used in combination with any other embodiments described herein.

1. A method for producing one or more oligosaccharides having at least three monosaccharide units, said method comprising the steps of co-culturing a first and a second genetically modified microbial cell in a culture medium, wherein,
a) the first genetically modified microbial cell is capable of producing a disaccharide or a first oligosaccharide of at least three monosaccharide units and wherein the genetically modified cell
   i) is capable of growing on a first carbon source while showing limited or no growth on a second carbon source, and
   ii) comprises one or more recombinant nucleic acid sequences encoding at least one glycosyltransferase; and
   iii) comprises at least one pathway to produce an activated sugar nucleotide from the first carbon source; and
   iv) is preferably capable of exporting said first oligosaccharide into the culture medium; and
b) the second genetically modified microbial cell is capable of producing a second oligosaccharide of at least three monosaccharide units and wherein the genetically modified cell
   i) is capable of growing on the second carbon source while showing limited or no growth on the first carbon source, and
   ii) comprises one or more recombinant nucleic acid sequences encoding at least one glycosyltransferase, and
   iii) comprises a biosynthetic pathway to produce an activated sugar nucleotide from the second carbon source
   iv) is preferably capable of exporting said second oligosaccharide into the culture medium.

2. The method according to item 1, wherein if a disaccharide is produced in step a) the disaccharide is not lactose.

3. The method according to item 1 or 2, wherein if a disaccharide is produced in step a) the disaccharide is lacto-N-biose (LNB) or N-acetyllactosamine (LacNAc).

4. The method according to item 1 to 3, wherein the first carbon source is selected from the group consisting of glucose, glycerol, sucrose, maltose, galactose, fructose, sorbitol, arabinose and maltose and the second carbon source is selected from sucrose, glycerol, galactose, maltose fructose, sorbitol, arabinose and glucose, and wherein the first and second carbon source are different.

5. The method according to any of the preceding, wherein one of the genetically modified microbial cells is capable of growing on sucrose and comprises one or more nucleic acid sequences encoding a PTS-dependent sucrose utilization system or a nucleic acid encoding a sucrose invertase or sucrose hydrolase enabling the assimilation of sucrose by said cell.

6. The method according to any one of item 5, wherein the PTS-dependent sucrose utilization system is encoded by scrY (SEQ ID NO: 97), scrA (SEQ ID NO: 98), scrB (SEQ ID NO: 99) and optionally scrR (SEQ ID NO: 100) or by the cscABKR-gene cluster (SEQ ID NO: 110) and the sucrose invertase is encoded by SacC_Agal (SEQ ID NO: 111) or Bff (SEQ ID NO: 112) or functional variants of any of these sequences.

7. The method according to item 5 or 6, wherein said cell has reduced or no ability to grow on one or more carbon sources selected from the group consisting of glucose, glycerol, galactose, maltose, sorbitol, arabinose and fructose.

8. The method according to item 1 to 3, wherein one of the genetically modified microbial cells is capable of growing on glucose and comprises one or more nucleic acids encoding one or more glucose transport systems.

9. The method according to item 8, wherein the glucose transport system is a PTS-dependent glucose transport system selected from the group consisting of:
i) glucose PTS complex components IICB^{Glc};
ii) beta-glucoside PTS complex components—IIABC^{Bgl};
iii) mannose PTS complex components—IICD^{Man};
iv) N-acetylglucosamine PTS complex components—IIABC^{Nag}; and
v) maltose/maltodextrin PTS complex—IICB^{malX}

10. The method according to item 8, wherein the glucose transport system is selected from the group consisting of:
i) galactose: H+ symporter GalP;
ii) glucose uptake protein GlcU;
iii) sodium/glucose transporter family (SGLT);
iv) galactose/glucose ABC transporter (mglABC) system;
v) trehalose/maltose/sucrose/palatinose (TMSP)-ABC transporter (malEFG) system;
vi) glucose/mannose ABC transporter (glcEFG) system;
vii) glucose proton symporter (glcP);
viii) glucose facilitator (glf), and
ix) hexose transporters (HXT).

11. The method according to item 8 to 10, wherein said cell has reduced or no ability to grow on one or more carbon sources selected from the group consisting sucrose, glycerol, galactose, maltose, sorbitol, arabinose and fructose.

12. The method according to item 1 to 3, wherein the genetically modified microbial cell capable of growing on glycerol comprises one or more nucleic acids encoding one or more glycerol transport systems.

13. The method according to item 12, wherein the glycerol transport system is selected from glycerol facilitator or glycerol/H+ symporter.

14. The method according to item 12 or 13, wherein said cell has reduced or no ability to grow on one or more carbon sources selected from the group consisting of sucrose, glucose, galactose, maltose, sorbitol, arabinose and fructose.

15. The method according to any one of the preceding items, wherein the first genetically modified microbial cell grows on sucrose and the second genetically modified microbial cell grows on glucose or glycerol.

16. The method according to any one of items 1 to 14, wherein the first genetically modified microbial cell grows on glucose or glycerol and the second genetically modified microbial cell grow on sucrose.

17. The method according to item 1 to 3, wherein the genetically modified microbial cell capable of growing on galactose comprises one or more nucleic acids encoding one or more galactose transport systems.

18. The method according to item 17, wherein the galactose transport system is selected from galactose:H+ symporter, the galactose/glucose ABC transporter (mglABC) system, the PTSLac (lacFE) system and/or the sodium/glucose transporter family (sglT).

19. The method according to item 17 or 18, wherein said cell has reduced or no ability to grow on one or more carbon sources selected from the group consisting of sucrose, glucose, glycerol, maltose, sorbitol, arabinose and fructose.

20. The method according to item 1 to 3, wherein the genetically modified microbial cell capable of growing on fructose comprises one or more nucleic acids encoding one or more fructose transport systems.

21. The method according to item 20, wherein the galactose transport system is selected from PTS complex components IIABC^{Fru}, glucose PTS complex components IICB^{Glc} and the fructose transporter FruP.

22. The method according to item 20 or 21, wherein said cell has reduced or no ability to grow on one or more carbon sources selected from the group consisting of sucrose, glucose, glycerol, maltose, sorbitol, arabinose and galactose.

23. The method according to item 1 to 3, wherein the genetically modified microbial cell capable of growing on maltose comprises one or more nucleic acids encoding one or more maltose transport systems.

24. The method according to item 23, wherein the galactose transport system is selected from MalFGK ABC superfamily transport system and/or maltose/maltodextrin PTS complex.

25. The method according to item 23 or 24, wherein said cell has reduced or no ability to grow on one or more carbon sources selected from the group consisting of sucrose, glucose, glycerol, fructose, sorbitol, arabinose and galactose.

26. The method according to item 1 to 3, wherein the genetically modified microbial cell capable of growing on arabinose comprises one or more nucleic acids encoding one or more arabinose transport systems.

27. The method according to item 26, wherein the arabinose transport system is selected from AraFGH ABC superfamily transport system and/or arabinose-proton symporter AraE.

28. The method according to item 26 or 27, wherein said cell has reduced or no ability to grow on one or more carbon sources selected from the group consisting of sucrose, glucose, glycerol, fructose, sorbitol, and galactose.

29. The method according to item 1 to 3, wherein the genetically modified microbial cell capable of growing on sorbitol comprises one or more nucleic acids encoding one or more sorbitol transport systems.

30. The method according to item 30, wherein the sorbitol transport system is selected from the sorbitol-PTS system (EIIBC^{srl}).

31. The method according to item 30 or 31, wherein said cell has reduced or no ability to grow on one or more carbon sources selected from the group consisting of sucrose, glucose, glycerol, fructose, arabinose and galactose.

32. The method according to any one of items 5 to 7, 12 to 14, 17 to 19, 20 to 22 or 23 to 25, wherein the functionality of one or more endogenous proteins involved in glucose import and utilization, in said cell are reduced or abolished, and wherein said proteins are selected from the group consisting of
i) glucose PTS complex components IICB^{Glc} (ptsG)
ii) beta-glucoside PTS complex components IIABC^{Bgl} (bglF)
iii) mannose PTS complex components—IICD^{Man}, (manX),
iv) N-acetylglucosamine PTS complex components IIABC^{Nag} (nagE)
v) maltose/maltodextrin transport system (malX)
vi) galactose/glucose high-affinity ABC transporter components (mglC)
vii) trehalose/maltose/sucrose/palatinose (TMSP)-ABC transporter malF;
viii) trehalose/maltose/sucrose/palatinose TMSP)-ABC transporter malG
ix) galactose permease (galP);
x) glucose proton symporter (glcP);
xi) glucose facilitator (glf);
xii) glucose uptake protein (glcU);
xiii) sodium/glucose transporter family (sglT)
xiv) hexose transporters (HXT); and/or
xv) glucokinase (glk).

33. The method according to item 32, wherein the functionality of the proteins involved in glucose and/or glycerol import and utilization are reduced or abolished by fully or partially inactivating of one or more of the gene genes selected from the group consisting of ptsG, bglF, manX, nagE, malX, malF, malG, mglC and glk.

34. The method according to any one of items 5 to 7, 8 to 11, 17 to 19, 20 to 22 or 23 to 25, wherein the functionality of the proteins involved in glycerol import and utilization are reduced or abolished by fully or partially inactivating of one or more of genes selected from the group consisting of glpF, stl1 and glpk.

35. The method according to any one of items 8 to 11, 12 to 14, 17 to 19, 20 to 22 or 23 to 25, wherein said cell does not contain genes encoding proteins involved in sucrose import and utilization, or wherein the functionality of one or more endogenous proteins in said cell is reduced or abolished, and wherein said proteins are selected from the group consisting of the PTS-dependent sucrose utilization system, in particular sucrose permeases, or sucrose invertase and sucrose hydrolase.

36. The method according to any one of items 5 to 7, 8 to 11, 12 to 14, 20 to 22 or 23 to 25, wherein the functionality of the proteins involved in galactose import and utilization are reduced or abolished by fully or partially inactivating of one or more of the genes selected from the group consisting of galP, lacF, mglA, mglC, sglT and galK.

37. The method according any one of items 5 to 7, 8 to 11, 12 to 14, 17 to 19 or 23 to 25, wherein the functionality of the proteins involved in fructose import and utilization are reduced or abolished by fully or partially inactivating of one or more of the genes selected from the group consisting of fruP, ptsG and fruA.

38. The method according to any one of items 5 to 7, 8 to 11, 12 to 14, 17 to 19 or 20 to 22 wherein the functionality of the proteins involved in maltose import and utilization are reduced or abolished by fully or partially inactivating of one or more of the genes selected from the group consisting of malF, malG, malK and malX.

39. The method according to item 38, wherein the functionality of the proteins involved in glucose import and utilization according to items 8 to 11 are also reduced or abolished by full or partial inactivation.

40. The method according to any one of the preceding items, wherein the one or more oligosaccharides are harvested from the co-culture.

41. The method according to any one of the preceding items, wherein the one or more oligosaccharides is one or more human milk oligosaccharides (HMOs).

42. The method according to item 41, wherein the one or more human milk oligosaccharides is a mixture of at least two human milk oligosaccharides.

43. The method according to anyone of the preceding items, wherein the one or more recombinant nucleic acid encoding at least one glycosyltransferase in the first and second genetically modified microbial cell independently is selected from the group consisting of β-1,3-N-acetyl-glucosaminyltransferase, beta-1,3-galactosyltransferase, beta-1,4-galactosyltransferase, alpha-1,2-fucosyltransferase, alpha-1,3-fucosyltransferase, alpha-1,3/4-fucosyltransferase, alpha-1,4-fucosyltransferase, alpha-2,3-sialyltransferase, and alpha-2,6-sialyltransferase.

44. The method according to any one of the preceding items, wherein the first and second genetically modified microbial cell independently are capable of producing one or more disaccharides or oligosaccharides selected from the group consisting of LNB, LacNAc, 2′FL, 3FL, 2′FLacNAc, 2′FLNB, Lewis A, Lewis X, 3′SL, 6′SL, 3′SLacNAc, 3′SLNB, sialyl-Lewis A, sialyl-Lewis X, DFL, LNT-II, LNT, LNnT, LNFP-I, LNFP-II, LNFP-IV, LNFP-V, LNFP-VI, FSL, LST-a, LST-b, LST-c, LST-d, LNDFH-II and LNDFH-III, DSLNT, pLNH, pLNnH, LNH, LNnH, (D)F-LNH-I, (D)F-LNH-II, (D)F-LNH-III, F-para-LNH-I, DF-para-LNH, DF-para-LNnH, TF-LNH, S-LNFP-I (FLST b), S-LNFP-II (FLST a), S-LNH, S-LNnH-I, FS-LNH, FS-LNnH-I, DS-F-LNH-II.

45. The method according to item 44, wherein one or more oligosaccharides are HMOs independently selected from the group consisting of 2′FL, 3FL, 3′SL, 6′SL, DFL, LNT-II, LNT, LNnT, LNFP-I, LNFP-III, LNFP-V, LNFP-VI, LST-a, LST-c, LNDFH-II and LNDFH-III.

46. The method according to any one of items 1 to 38 or 40 to 45, wherein the first genetically modified microbial cell comprises a beta-1,4-galactosyltransferase allowing galactosylation of a free glucose monosaccharide to intracellularly generate lactose and wherein the glucokinase activity, converting glucose into glucose-6-phosphate, in said cell is reduced or abolished.

47. The method according to any one of the preceding claims, wherein said the first genetically modified microbial cell is capable of producing LacNAc, LNB, Lewis A, Lewis X, 2′FL, 3FL LNT-II, LNT, LNnT LNFP-I, LST-c or LST-a preferably without the addition of lactose to the culture medium.

48. The method according to claim 47, wherein the HMO produced by the first genetically modified cell is the most abundant HMO produced by said cell.

49. The method according to any one of the preceding items, wherein the first genetically modified microbial cell comprises a recombinant nucleic acid sequence encoding a transporter protein capable of exporting the disaccharide or first oligosaccharide product into the extracellular medium.

50. The method according to item 49, wherein the transporter protein is a sugar efflux transporter or a major facilitator superfamily (MFS) transporter, preferably selected from the group consisting of setA, yberC, nec, vag, marc, bad and fred.

51. The method according to any one of the preceding items, wherein the first genetically modified microbial cell produces the intermediate product LNT-II and comprises
a) a recombinant nucleic acid sequence encoding a beta-1,3-N-acetylglucosaminyltransferase, and
b) a biosynthetic pathway for making UDP-GlcNAc from the carbon source assimilated by the first genetically modified microbial cell, and
c) optionally a recombinant nucleic acid sequence encoding a MFS transporter capable of exporting LNT-II into the extracellular medium.

52. The method according to any one of the preceding items, wherein the first genetically modified microbial cell produces the intermediate product LNT and comprises
a) a recombinant nucleic acid sequence encoding a beta-1,3-N-acetylglucosaminyl transferase, and
b) a recombinant nucleic acid sequence encoding a beta-1,3-galactosyltransferase, and
c) biosynthetic pathways for making UDP-GlcNAc and UDP-Gal from the carbon source assimilated by the first genetically modified microbial cell, and
d) optionally a recombinant nucleic acid sequence encoding a MFS transporter capable of exporting LNT into the extracellular medium.

53. The method according to any one of the preceding items, wherein the first genetically modified microbial cell produces the intermediate product LNnT and comprises
a) a recombinant nucleic acid sequence encoding a beta-1,3-N-acetylglucosaminyl transferase, and
b) a recombinant nucleic acid sequence encoding a beta-1,4-galactosyltransferase, and
c) biosynthetic pathways for making UDP-GlcNac and UDP-Gal from the carbon source assimilated by the first genetically modified microbial cell, and
d) optionally a recombinant nucleic acid sequence encoding a MFS transporter capable of exporting LNnT into the extracellular medium.

54. The method according to any one of the preceding items, wherein the first genetically modified microbial cell produces the intermediate product 2′-FL or 3-FL and comprises
a) a recombinant nucleic acid sequence encoding alpha-1,2-fucosyltransferase or alpha-1,3-fucosyltransferase, and
b) a biosynthetic pathways for making GDP-fucose from the carbon source assimilated by the first genetically modified microbial cell, and
c) optionally a recombinant nucleic acid sequence encoding a MFS transporter capable of exporting 2′-FL or 3-FL into the extracellular medium, and
d) optionally a beta-1,4-galactosyltransferase allowing galactosylation of a free glucose monosaccharide to intracellularly generate lactose.

55. The method according to any one of the preceding items, wherein the first genetically modified microbial cell produces the intermediate product 3′SL or 6′SL and comprises
a) a recombinant nucleic acid sequence encoding α-2,3-sialyltransferases, or α-2,6-sialyltransferases, and
b) a biosynthetic pathways for making CMP-N-acetylneuraminic acid (CMP-Neu5Ac) from the carbon source assimilated by the first genetically modified microbial cell, and
c) optionally a recombinant nucleic acid sequence encoding a MFS transporter capable of exporting 3′SL or 6′SL into the extracellular medium, and
d) optionally a beta-1,4-galactosyltransferase allowing galactosylation of a free glucose monosaccharide to intracellularly generate lactose.

56. The method according to any one of the preceding items, wherein the second genetically modified microbial cell comprises at least one nucleic acid sequence encoding a protein or protein complex which is capable of importing the disaccharide or oligosaccharide produced by said first genetically modified microbial cell.

57. The method according to item 56, the protein or protein complex which is capable of importing the disaccharide or oligosaccharide produced by said first genetically modified microbial cell is selected from table 1 or 2.

58. The method according to any one of the preceding items, wherein the second genetically modified microbial cell comprises
a) one or more recombinant nucleic acid sequences encoding a glycosyltransferase selected from the group consisting of beta-1,3-galactosyltransferase, beta-1,4-galactosyltransferase, alpha-1,2-fucosyltransferase, alpha-1,3-fucosyltransferase, alpha-1,3/4-fucosyltransferase, alpha-1,4-fucosyltransferase, α-2,3-sialyltransferases, and α-2,6-sialyltransferases, and
b) optionally a non-functional or deleted lactose permease, and
c) optionally a recombinant nucleic acid sequence encoding a MFS transporter capable of exporting the oligosaccharide produced by the second genetically modified microbial cell.

59. The method according to item 58, wherein the MFS transporter is selected from marc, nec, yberC and vag.

60. The method according to any one of items 56 to 59, wherein the one or more HMOs produced by the second genetically modified microbial cell has at least three, such as at least four monosaccharide units and are selected from the group consisting of Lewis A, Lewis X, sialyl-LacNAc, sialyl-LNB, sialyl-Lewis X, sialyl-Lewis A, Lewis B, Lewis Y, DFL, FSL, LNT, LNnT, LST-a, LST-b, LST-c, LST-d, LNFP-I, LNFP-II, LNFP-III, LNFP-V, LNFP-VI, LNDFH-I, LNDFH-II, LNDFH-III, DSLNT, pLNH, pLNnH, LNH, LNnH, (D)F-LNH-I, (D)F-LNH-II, (D)F-LNH-III, F-para-LNH-I, DF-para-LNH, DF-para-LNnH, TF-LNH, S-LNFP-I (FLST b), S-LNFP-II (FLST a), S-LNH, S-LNnH-I, FS-LNH, FS-LNnH-I, DS-F-LNH-II, or a mixture of these.

61. The method according to any one of the preceding items, wherein the one or more oligosaccharides produced are harvested from the cell culture.

62. The method according to any one of items 1 to 50, wherein the first genetically modified microbial cell according to item 54 is cultured with a second genetically modified microbial cell modified microbial cell comprising
a) a recombinant nucleic acid sequence encoding a transporter protein capable of importing the oligosaccharide from the first genetically modified microbial cell, and
b) one or more recombinant nucleic acid sequences encoding a glycosyltransferase selected from the group consisting of alpha-1,2-fucosyltransferase, alpha-1,3-fucosyltransferase, alpha-1,3/4-fucosyltransferase, α-2,3-sialyltransferases, and
c) optionally a recombinant nucleic acid sequence encoding a MFS transporter, and
d) where the HMO product produced by second genetically modified microbial cell is DFL or FSL.

63. The method according to item 62, wherein the nucleic acid sequence encoding a transporter protein capable of importing the intermediate product is a lactose permease capable of importing fucosyllactose, preferably the lactose permease is overexpressed.

64. The method according to item 62 or 63, wherein the MFS transporter is selected from the group consisting of nec, marc, fred, setA and bad. 65. A method for producing LNT or LNnT, said method comprising co-culturing
a) a first genetically modified microbial cell according to item 51, and
b) a second genetically modified microbial cell according to any one of items 56 to 59, wherein
   i) the nucleic acid sequence encoding a protein or protein complex capable of importing the oligosaccharide produced by said first genetically modified microbial cell is recombinant and encodes a transporter protein capable of importing LNT-II, selected from the group consisting of a mutant lacY-transporter, MFS transporter, such as Blon_0962 and an ABC transporter, such as Blon_2177, 2176, 2175 or Blon_0883-0884-0885-08836, BBPC_1775-1776-1777 and Bbr_0527-0528-0530-0531 or RHOM_04095-04100-04105; and,
   ii) the recombinant nucleic acid sequences encoding a glycosyltransferase encodes a beta-1,4-galactosyltransferase or a beta-1,3-galactosyltransferase, and
   iii) the recombinant nucleic acid encoding the MFS transporter capable of exporting the oligosaccharide encodes vag, nec, Edic1 or YberC, and
c) harvesting the LNnT produced in the co-culture, and wherein the method produces significantly less LNT-II and/or minimal or no pLNnH by-product compared to LNnT produced by a single cell.

66. A method for producing LNFP-I, LNFP-II, LNFP-V, LNDFH-I and/or LNDFH-II, said method comprising co-culturing
a) a first genetically modified microbial cell according to item 52, and
b) a second genetically modified microbial cell according to any one of items 56 to 59, wherein said cell comprises
   i) the nucleic acid sequence(s) encoding a protein or protein complex capable of importing the oligosaccharide produced by said first genetically modified microbial cell is recombinant and encodes a transporter protein capable of importing LNT, and
   ii) the recombinant nucleic acid sequences encoding a glycosyltransferase encodes a fucosyltransferase selected from the group consisting of alpha-1,2-fucosyltransferase, alpha-1,3-fucosyltransferase, alpha-1,3/4-fucosyltransferase, alpha-1,4-fucosyltransferase, and
c) harvesting the LNFP-I, LNFP-II, LNFP-V, LNDFH-I and/or LNDFH-II produced in the co-culture, wherein the method produces significantly less LNT, pLNH2 and LNT-II by-product as well as and fucosylated derivatives thereof compared to LNFP-I, LNFP-II and/or LNFP-V produced by a single cell.

67. A method for producing LNFP-III, LNFP-VI and/or LNDFH-III, said method comprising co-culturing
a) a first genetically modified microbial cell according to item 53, and
b) a second genetically modified microbial cell according to any one of items 56 to 59, wherein said cell comprises
   i) the nucleic acid sequence(s) encoding a protein or protein complex capable of importing the oligosaccharide produced by said first genetically modified microbial cell is recombinant and encodes a transporter protein capable of importing LNnT, and
   ii) the recombinant nucleic acid sequences encoding a glycosyltransferase encodes an alpha-1,3-fucosyltransferase or alpha-1,3/4-fucosyltransferase, and
c) harvesting the LNFP-III, LNFP-VI and/or LNDFH-III produced in the co-culture, wherein the method produces significantly less LNnT, pLNH2 and LNT-II by-product as well as and fucosylated derivatives thereof compared to LNFP-III, LNFP-VI and/or LNDFH-III produced by a single cell.

68. The method according to any one of items 1 to 50, wherein the one or more oligosaccharides produced by said method is a oligosaccharide produced from a donor oligosaccharide and an acceptor oligosaccharide produced by a first and a second genetically modified cell and said method further comprises the steps of:
a) making an enzyme with transglycosidase activity available in the culture medium, and
b) incubating the disaccharide or first oligosaccharide, with the second oligosaccharide produced in the co-culture with the transglycosidase enzyme in the culture medium to form a third oligosaccharide in the culture medium.

69. The method according to item 68, wherein the oligosaccharide produced is a complex oligosaccharide of at least 4 such as at least five monosaccharide units.

70. The method according to any one of items 1 to 50, wherein the one or more oligosaccharides produced by said method is a sialylated and/or a fucosylated oligosaccharide of at least four monosaccharide units, and wherein at least the first or the second genetically modified microbial cell produces a sialylated or fucosylated donor oligosaccharide and the other cell produces an acceptor oligosaccharide and said method further comprises the steps of:
a) making an enzyme with transglycosidase activity available in the culture medium, wherein the enzyme with transglycosidase activity is
   i) a transfucosidase if the donor oligosaccharide is a fucosylated oligosaccharide, or
   ii) a transsialidase if the donor oligosaccharide is a sialylated oligosaccharide, and
b) incubating the first oligosaccharide, the second oligosaccharide produced in the co-culture with the transglycosidase enzyme in the culture medium to form a third oligosaccharide of at least four monosaccharide units in the culture medium.

71. The method according to item 68, wherein the transglycosidase enzyme is either added to the culture medium during the cultivation or is expressed from a recombinant nucleic acid in one of the genetically modified cells or from a third genetically modified cell in the same culture medium as the first and second genetically modified strains.

72. The method according to item 71, wherein the third genetically modified cell grow on one of the carbon sources already present in the culture.

73. The method according to item 71, wherein the third genetically modified cell grow on a third carbon source.

74. The method according to item 68 to 73, wherein the transglycosidase enzyme is selected from the group consisting of α-1,2-tranfucosidase, α-1,3-transfucosidase, α-1,3/4-transfucosidase, α-2,3-transialylase, α-2,6-transsialylase, trans-lacto-N-biosidase, β-N-acetylglucosaminidase and trans-β-galactosidase.

75. The method according to any one of items 68 to 74, wherein the first and the second genetically modified microbial cells comprise a lactose importer, such as a lactose permease.

76. The method according to any one of items 68 to 75, wherein lactose is added to the co-culture as substrate for producing the first and second oligosaccharide.

77. The method according to any one of items 68 or 71 to 76, wherein the donor oligosaccharide is selected from the group consisting of LNT, LNFP-I and LST-a.

78. The method according to any one of items 68 to 76, wherein the donor oligosaccharide is selected from the group consisting of 2′FL, 3FL, DFL, LNT, LNnT, FSL, sialyl-LacNAc, sialyl-LNB, 3′SL and 6′SL.

79. The method according to item 77, wherein the donor oligosaccharide is produced by a cell according to item 54 or 55.

80. The method according to any one of items 68 to 77, wherein the acceptor oligosaccharide is selected from the group consisting of LNB, LacNAc, Lewis A, Lewis X, 2′FL, 3-FL, 3′SL, LNT, LNnT, LNFP-I, LNFP-II, LNFP-III, LNFP-V, LNFP-VI, LST-a and LST-c.

81. The method according to item 68 to 80, wherein the donor oligosaccharide and the acceptor oligosaccharide are different.

82. The method according to any one of items 68 to 81, wherein the one or more oligosaccharide produced by incubating the transsialidase enzyme with the donor and acceptor molecules has at least three monosaccharide units, such as at least four monosaccharide units, and are selected from the group consisting of Lewis X, Lewis A, 3′SLacNAc, 3′SLNB, 6′SLNB, 6′SlacNAc, GlcNAc(1-3)-3FL, Lewis Y, Lewis B, sialyl-Lewis X, sialyl-Lewis A, DFL, LNFP-I, LNFP-II, LNFP-III, LNFP-V, LNFP-IV, LNDFH-I, LNDFH-II, LNDFH-III, DF-para-LNnH, FLSTa (S-LNFP-II), FSL, LSTa, FLSTa, LSTc, FLSTc, 6′SLN, FLSTb (S-LNFP-I), LSTb, DSLNT, para-LNH, gal-LNnT, F-p-LNH, S-p-LNH.

83. The method according to any one of the preceding claims, wherein the one or more oligosaccharides produced are harvested from the cell culture.

84. The method according to any of the preceding items, wherein the first and second genetically modified microbial cell is selected from the group consisting of Escherichia coli, Corynebacterium glutamicum, Lactococcus lactis, Bacillus subtilis, Streptomyces lividans, Pichia pastoris and Saccharomyces cerevisiae.

85. The method according to item 84, wherein the first and second genetically modified microbial cell are selected from the same species.

86. The method according to item 84 or 85, wherein the genetically modified microbial cell is Escherichia coli.

87. Use of one or more oligosaccharides produced by a method according to claims 1 to 86 in the production of a nutritional composition.

### EXAMPLES

### Background Strain—MDO

The strains (genetically engineered cells) constructed in the present application were based on Escherichia coli K-12 DH1 with the genotype: F⁻, k⁻, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, me/A: deletion of 0.9 kbp, wcaJ: deletion of 0.5 kbp, mdoH: deletion of 0.5 kbp, and insertion of Plac promoter upstream of the gmd gene. The MDO strain is used as background strain in all the following examples.

Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186: 2673-81 and Warming et al 2005 Nucleic Acids Res. 33(4): e36) with specific selection marker genes and screening methods.

### Example 1: Co-Fermentation of Two Strains Producing a HMO Mixture by Co-Culturing a 3′SL and a LNT Producing Strain

In the present example it is illustrated that two strains growing on different carbon sources can be co-cultured to produce a mixture of HMOs.

### Strains

The MDO strain described above was further engineered to generate a 3′SL capable of growing on glucose producing strain and an LNT producing strain with the genotypes shown in table 14.

TABLE 14
genotypes of strains used in the examples

| Strain | | Carbon | |
|---|---|---|---|
| ID | Product | Source | Genotype |
| MDO | None | +Glucose | F-λ- ΔendA1 ΔrecA1 ΔrelA1 ΔgyrA96 Δthi-1 glnV44 |
| | | ↓Sucrose | hsdR17(rK-mK-) ΔlacZ wcaF::Plac ΔnanKETA ΔlacA ΔmelA |
| | | | ΔwcaJ ΔmdoH |
| MF1 | 3′SL | +Glucose | MDO + 2xPlac-D29nst¹ ΔnadC² pBS-nadC-Plac-neuBCA³ |
| | | ↓Sucrose | Δlacl¹⁰ |
| MF2 | LNT | +Sucrose | MDO + 3x PglpF-IgtA⁴ 2x PglpF-galTK⁵, PglpF_SD1-scrYA⁶ |
| | | ↓Glucose | Pscr-scrBR⁶, Plac-yberC⁷ PglpF-lacY⁸ ΔptsG⁹ Δglf¹⁰ Δlacl¹¹ |

¹D29nst - gene encoding α-2,3-sialyltransferase of SEQ ID NO: 91 under control of the Plac promoter (SEQ ID NO: 92).
²DnadC - deletion of quinolinate phosphoribosyl-transferase of WP_101348535.1 for further details see WO2017101958.
³pBS-nadC-Plac-neuBCA - plasmid expressing neuBCA (SEQ ID NO: 94) and nadC further details see WO 2017/101958.
⁴lgtA - three genomically inserted copies of a gene encoding β-1,3-N-acetyl-glucosaminyltransferase (SEQ ID NO: 94) under control of a PglpF promoter (SEQ ID NO: 93).
⁵galTK - two genomically inserted gene encoding β-1,3-Galactosyltransferase (SEQ ID NO: 96) under control of a PglpF promoter (SEQ ID NO: 93).
⁶scrYA, scrBR two operons encoding the sequences of SEQ ID NO: 97 and 98 and SEQ ID NO: 99 and 100 respectively, and under the control of the PglpF_SD1 promoter (SEQ ID NO: 101) and Pscr promoter (SEQ ID NO: 102), respectively.
⁷YberC - genomically integrated MFS transporter (SEQ ID NO: 103) under control of the Plac promoter (SEQ ID NO_92)
⁸lacY - additional genomically integrated copy of lacY (SEQ ID NO: 1) under control of a PglpF promoter (SEQ ID NO: 93).
⁹ΔptsG - deletion of the PTS system glucose-specific EIICB component to limit growth on glucose
¹⁰Δglf - deletion of the native glucose facilitator - to limit growth on glucose.
¹¹Δlacl - Deletion of lac repressor makes use of IPTG obsolete.

### Fermentation

The E. coli strains were cultivated in a bioreactor with mineral culture medium consisting of 15 g/L glucose, 15 g/L sucrose, lactose monohydrate, (NH₄)₂HPO4, KH₂PO₄, MgSO₄×7H₂O, KOH, NaOH, citric acid, trace element solution, antifoam and thiamine. The dissolved oxygen level was kept at 20% by a cascade of first agitation and then airflow starting at 1000 rpm (up to max 2000 rpm) and 1 VVM (up to max 3 VVM). The pH was kept at 6.8 by titration with 10% NH₄OH solution. The cultivation was started with 1% (v/v) inoculums from each strain from pre-cultures grown in a similar glucose (3′SL strain) or sucrose (LNT strain) containing medium. After depletion of the glucose and sucrose contained in the batch medium, a feed solution containing 1:1 ratio of (w/w) of glucose and sucrose (sterilized together separately from the minerals), MgSO₄×7H₂O, trace metals and antifoam was fed continuously using a constant profile that kept the culture carbon limited. The temperature was initially at 34° C. but was dropped to 28° C. with a 1 h ramp after 3 hours of feed. The growth and metabolic activity and state of the cells were followed by on-line measurements of agitation, ammonium hydroxide base addition, temperature, pH, respiratory quotient and CO₂ evolution rate.

### Results

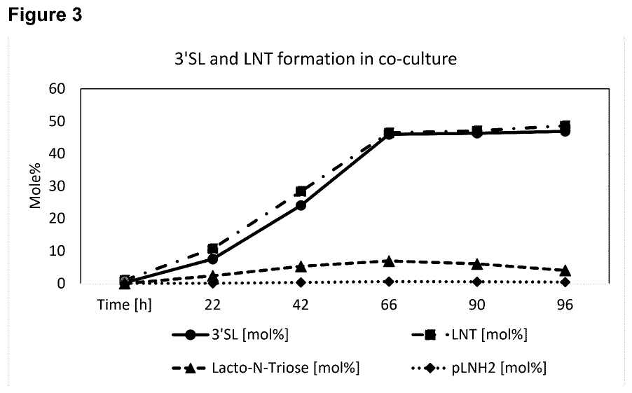

The results of the fermentation are shown in table 15 and FIG. 3 as for the mole % of individual HMOs of the % of total HMO (mM)+lactose.

TABLE 15
Formation of HMO in a co-fermentation of a 3′SL and LNT strain

| Time | 3′SL | LNT | LNT/3′SL ratio | LNT-II | pLNH2 |
|---|---|---|---|---|---|
| [h] | [mol %] | [mol %] | [mol/mol] | [mol %] | [mol %] |
| 22 | 0.26 | 1.04 | 4.07 | 0.00 | 0.00 |
| 42 | 7.54 | 10.71 | 1.42 | 2.34 | 0.09 |
| 66 | 24.09 | 28.38 | 1.18 | 5.31 | 0.32 |
| 90 | 45.96 | 46.49 | 1.01 | 6.95 | 0.60 |
| 96 | 46.31 | 47.09 | 1.02 | 6.07 | 0.53 |
| 114 | 46.91 | 48.63 | 1.04 | 4.04 | 0.43 |

From table 15 it can be seen that LNT is produced slightly faster than 3′SL for the first 42 hours of the fermentation. For the reminder of the fermentation the production of the two molecules is close to a 1:1 ratio, which is also illustrated in FIG. 3, where the production of 3′SL and LNT follow very similar curves. This is a clear indication that it is possible to control the growth and production of each strain by the controlled addition of two different carbon sources and that one strain could not outcompete the other.

This essentially provide the option of producing a controlled mixture of HMOs in a single fermentation which has the potential benefit of merging production of different products whose volumes on their own don't warrant a full batch.

### Example 2 Synthesis of LST-c Using a Two Strains Hybrid Process

In the present example two strains, one producing LNnT from lactose (substrate) and sucrose (carbon source) and the second producing 6′SL from lactose (substrate) and glucose (carbons source) were co-cultured. 6′SL was produced as sialyl donor substrate and LNnT as acceptor substrate for a subsequent trans-sialyation reaction. LST-c was formed in the fermentation medium by addition of a α2,6-transsialidase to the fermentation medium. This process is also termed a two-strain hybrid process.

### Strains

The MDO strain described in the background strain section above was further engineered to generate a 6′SL producing strain and a LNnT producing strain with the genotypes shown in table 16.

TABLE 16
genotypes of strains used in the examples

| Strain ID | Product | Carbon Source | Genotype |
|---|---|---|---|
| MDO | None | +Glucose | F-λ- ΔendA1 ΔrecA1 ΔrelA1 ΔgyrA96 Δthi-1 glnV44 |
| | | ↓Sucrose | hsdR17(rK-mK-) ΔlacZ wcaF::Plac ΔnanKETA ΔlacA |
| | | | ΔmelA ΔwcaJ ΔmdoH |
| MF5 | 6′SL | +Glucose | MDO PglpF-ST6, Pd2¹ ΔnadC² pBS-nadC-Plac-neuBCA³ |
| | | ↓Sucrose | |
| MF6 | LNnT | +Sucrose | MDO 2x PglpF-lgtA⁴, -PglpF-galT⁵, PglpF_SD1-Pscr- |
| | | ↓Glucose | scrYA, Pscr-scrBR, PglpF-vag⁷, ΔptsG⁸ Δlacl⁹ |

^{1ST}6, Pd2 - gene encoding a2,6-sialyltransferase of SEQ ID NO: 104.
²DnadC - deletion of quinolinate phosphoribosyl-transferase of WP_101348535.1 for further details see WO2017/101958.
³pBS-nadC-Plac-neuBCA - plasmid expressing neuBCA (SEQ ID NO: 94) and nadC further details see WO 2017/101958.
⁴lgtA-PglpF - two genomically inserted copies of a gene encoding β-1,3-N-acetyl-glucosaminyltransferase (SEQ ID NO: 95) under control of a PglpF promoter (SEQ ID NO: 93).
⁵galT - one genomically inserted gene encoding β-1,4-Galactosyltransferase (SEQ ID NO: 105) under control of a PglpF promoter (SEQ ID NO: 93).
⁶scrYA, scrBR two operons encoding the sequences of SEQ ID NO: 97 and 98 and SEQ ID NO: 99 and 100 respectively, and under the control of the PglpF_SD1 promoter (SEQ ID NO: 101) and Pscr promoter (SEQ ID NO: 102), respectively.
⁷vag gene coding for a heterologous major facilitator superfamily (MFS) transporter of SEQ ID NO: 106 under control of a PglpF promoter (SEQ ID NO: 93).
⁸ΔptsG - deletion of thePTS system glucose-specific EIICB component to limit growth on glucose
⁹Δlacl - Deletion of lac repressor makes use of IPTG obsolete.

### Fermentation

The 2-strain hybrid process was carried out in a 2 L Sartorious B-stat bioreactor starting with 1000 g of mineral culture medium consisting of 15 g/kg glucose (sterilized separately) and 15 g/kg sucrose (sterilized separately), lactose monohydrate (sterilized separately) in an amount sufficient to produce the desired amount of oligosaccharide produced by the cell, however not more than what the cell can convert into the desired oligosaccharide produced by the cell, e.g. in the range from 10-80 g/kg, (NH₄)₂HPO4, KH₂PO₄, MgSO₄×7H₂O (sterilized separately), KOH, NaOH, citric acid, trace element solution, antifoam and thiamine (filter sterilized). The dissolved oxygen level was kept at 20% by a cascade of first agitation and then airflow of 1 VVM (up to max 3 VVM). The pH was kept at 6.8 by titration with NH₄OH solution. The cultivation was started with 1% (v/v) inoculums from each pre-culture grown in a similar containing medium to an OD₆₀₀ of 2.5-5, with glucose as carbon source for the 6′SL strain and sucrose as carbon source for the LNnT strain.

After depletion of the glucose and sucrose contained in the batch medium after approximately 14 h, a feed solution containing glucose and sucrose at a 1:2 ratio (w/w) (sterilized together separately from the minerals), MgSO₄×7H₂O, H₃PO₄, trace metals and antifoam, was fed continuously using a constant profile that kept the culture carbon limited. The temperature was initially set to 34° C. and was dropped to 28° C. with a linear 1 h ramp after 3 hours of feed. The growth, and state of the cells were followed by biowetmass (weight of cell pellet/weight of broth after 3 min centrifugation at 14,000 g), optical density at 600 nm and on-line measurements of CO₂ evolution rate, agitation, base addition, dissolved oxygen and temperature.

The transsialylation was started by adding 3.39 mg/ml of α-2,6-transsialidase from Photobacterium 83eiognathid JT-SHIZ-119 (PITS-197, SEQ ID NO: 88) at 113 h after fermentation start, when almost all the lactose had been converted into 6′SL and LNnT. The process was monitored via HPLC by measuring the concentrations of substates and product. Lactose and oligosaccharide concentrations were determined once or several times per day via HPLC. Samples for HPLC analysis were heat treated at 90° C. for 20 min at the time of collection to stop the enzymatic process.

### Results

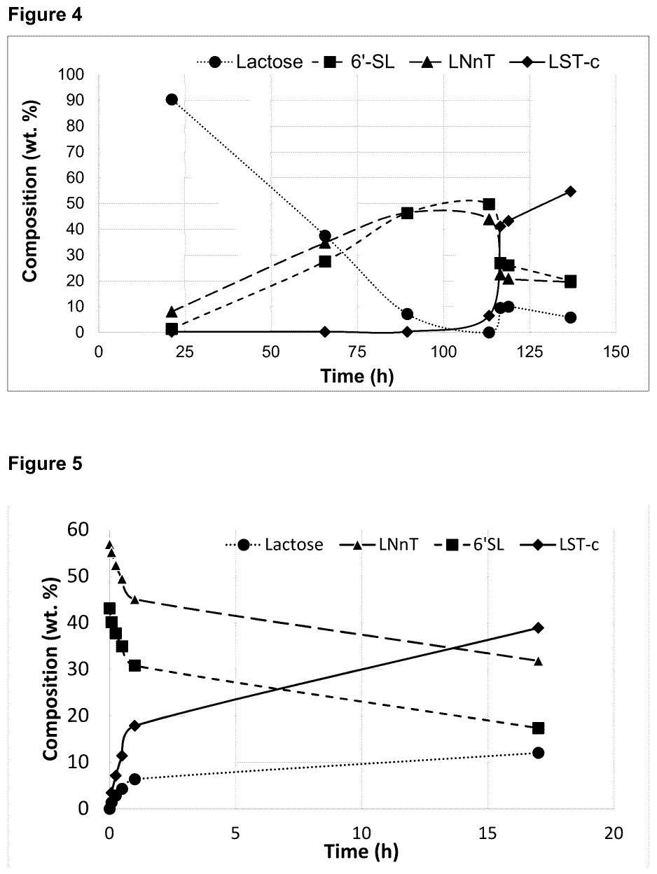

FIG. 4 shows concentration data illustrating the process progress in weight % relative to the total weight of substrates and products.

From this it can be seen, that up until 113 h where all the lactose is consumed and the transsialidase was added to the culture, the LNnT and 6′SL strains produced 6′SL and LNnT in close to equimolar ratios. Upon the addition of the transsialidase the LST-c formation was initiated by consuming 6′SL and LNnT as can be seen by their decreasing amounts. The formation of LST-c also generates an equimolar amount of lactose as the leaving group from the transsialylation reaction. However, the lactose is taken up by the LNnT and 6′SL strains and reutilized to form 6′SL and LNnT.

This example in addition to example 1 shows that the stable formation of two different products obtained by co-culturing of two strains on two different carbon sources can be utilized in a further process (trans-sialylation) to form a third product (see FIG. 4). It also shows that large differences in product yields (g LNnT or 6′SL/g carbon source) of two strains can be compensated by feeding them at a modified ratio to achieve a desired product ratio, in this case a glucose:sucrose ratio of 1:2 (g/g) to achieve a ratio of 6′SL:LNnT of 1:1.

To illustrate the difference of the two-strain hybrid system, a conventional in-vitro enzymatic process was conducted as well using the α-2,6-transsialidase PITS-197 to catalyze transsialylation of LNnT utilizing 6′SL as a sialyl donor. A substrate solution was prepared with 116.8 mM LNnT and 116.8 mM 6′SL at pH 6.87. The transsialylation reaction was started by adding 1.57 mg/ml PITS-197 at 25° C. The reaction progress was monitored by measuring the concentrations of substrates and products via HPLC. Samples for HPLC measurements were collected by treating them at 95° C. for 5 min to denature the PITS-197 enzyme thereby stopping the reaction. FIG. 5 shows the reaction progress curve of the in-vitro synthesis of LST-c. As can be seen in FIG. 5 in contrast to the two-strain hybrid process which lead to a very low lactose concentration (shown at FIG. 4), the in-vitro process leads to an equimolar amount of lactose with LST-c. Table 17 shows comparison of the final product composition of LST-c synthesis using hybrid vs. in-vitro processes.

TABLE 17
Comparison of final product composition of the two-strain hybrid
process vs. in-vitro process for the synthesis of LST-c starting
at an acceptor (LNnT) to donor (6′SL) in equimolar ratio.

| | Lactose | 6′-SL | LNnT | LST-c |
|---|---|---|---|---|
| Process | (wt. %) | (wt. %) | (wt. %) | (wt. %) |
| In-vitro | 11.9 | 17.3 | 31.8 | 38.8 |
| Hybrid | 5.8 | 20.0 | 19.5 | 54.6 |

From table 17 it can be seen that the LST-c formation was significantly increased in the two-strain hybrid process resulting from a higher conversion of LNnT. In addition, the final product mixture obtained from the two-strain hybrid process had a lower level of lactose. Thus, the two-strain hybrid process offers the advantage of obtaining higher levels of LST-c and reduced lactose levels. In addition, the hybrid process offers the advantage of replacing the expensive starting materials 6′SL and LNnT with cheap lactose and offering a higher conversion of LNnT to the desired product LST-c compared to the in vitro process, thus in all constituting a much more economical process.

### Example 3: Production of DFL by Co-Culturing 2′FL and a 3FL Strain

In the present example a 2′FL strain capable of growing on sucrose was co-cultured with a 3FL producing strain growing on glucose. It shows how 2′-Fucosyllactose (2′FL) produced by the 2′FL strain was taken up by the 3-fucosyllactose (3FL) strain where an additional fucose is added to the 3-position of 2′FL to form 2′,3-Difucosyllactose (DFL).

### Strains

The MDO strain described in the background strain section above was further engineered to generate a 2′FL producing strain and a 3FL producing strain with the genotypes shown in table 18.

TABLE 18
genotypes of strains used in this example

| Strain | | Carbon | |
|---|---|---|---|
| ID | Product | Source | Genotype |
| MDO | None | +Glucose | F-λ- ΔendA1 ΔrecA1 ΔrelA1 ΔgyrA96 Δthi-1 glnV44 |
| | | ↓Sucrose | hsdR17(rK-mK-) ΔlacZ wcaF::Plac ΔnanKETA ΔlacA |
| | | | ΔmelA ΔwcaJ ΔmdoH |
| MF3 | 2′FL | +Sucrose | MDO 2xPglpF-futC¹ PglpF-CA² PglpF-nec³ Pscr-scrBR⁴ |
| | | ↓Glucose | PglpF_SD1-scrYA⁴ ΔglpR⁸ ΔptsG⁹ |
| MF4 | 3FL and | +Glucose | MDO PglpF-CA² 3xPglpF-futA⁵ PglpF-marc⁶ |
| | DFL | ↓Sucrose | |

¹futC - gene encoding alpha-1,2-fucosyl-transferase of SEQ ID NO: 108.
²CA = extra colanic acid gene cluster (gmd-wcaG-wcaH-wcal-manC-manB, SEQ ID NO: 109) under the control of a PglpF promoter at a locus that is different than the native locus.
³nec gene coding for a heterologous major facilitator superfamily (MFS) transporter of SEQ ID NO: 107, under the control of a PglpF promoter (SEQ ID NO: 93).
⁶scrYA, scrBR two operons encoding the sequences of SEQ ID NO: 97 and 98 and SEQ ID NO: 99 and 100 respectively, and under the control of the PglpF_SD1 promoter (SEQ ID NO: 101) and Pscr promoter (SEQ ID NO: 102), respectively.
⁵futA - three independent genomic copies of the gene encoding α-1,3-fucosyl-transferase of SEQ ID NO: 90 under control of the PglpF promoter (SEQ ID NO: 93).
⁶marc gene coding for a heterologous major facilitator superfamily (MFS) transporter of SEQ ID NO: 90.
⁷lacY - additional genomically integrated copy of lacY (SEQ ID NO: 1) under control of a PglpF promoter (SEQ ID NO: 93).
⁸ΔglpR - Deletion of glpR repressor.
⁹ΔptsG - deletion of the PTS system glucose-specific EIICB component to limit growth on glucose.

### Fermentation

The 2-strain fermentation process was carried in the same way as in example 1. The cultivation was started with 1% (v/v) inoculums from each strain from pre-cultures grown in a similar glucose (3FL strain) or sucrose (2′FL strain) containing medium.

### Results

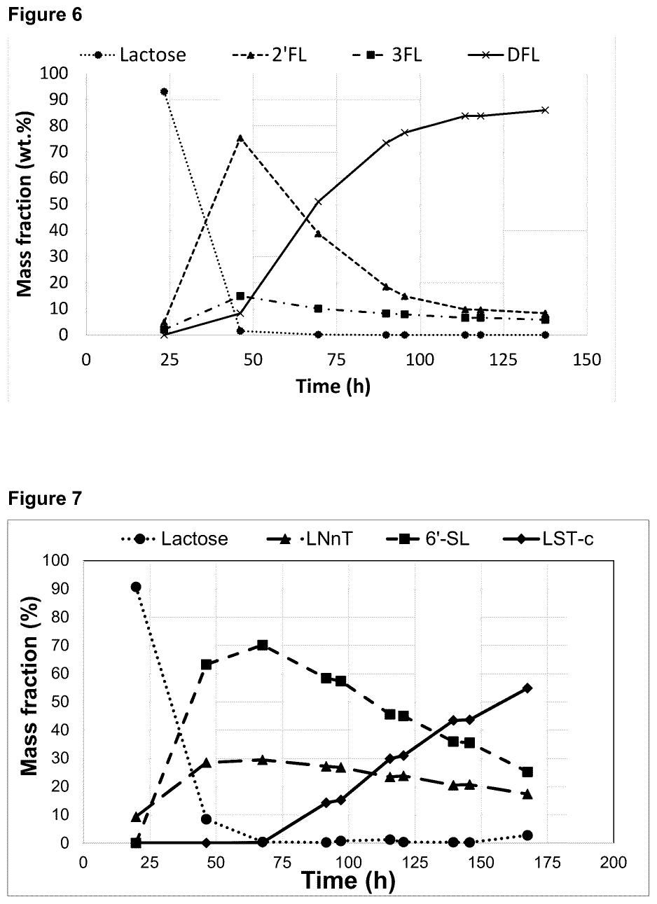

FIG. 6 shows the process progress in weight % relative to the total weight of substrates and products.

From this it can be seen that 2′FL was produced far in excess of 3FL up until the point where all the lactose had been consumed, reflecting the different carbon source yields of the strains. DFL had already started to accumulate at that time but was still a minority product. However, once the lactose had been depleted, the DFL formation rate accelerated greatly. In the end the final composition was 86 wt % DFL, 8.3 wt % 2′FL and 5.8% 3FL showing the feasibility to produce DFL as a major product by the co-culturing of a 2′FL and a 3FL strain.

This example shows how one product can be formed from one strain making a precursor molecule that is exported to the medium and subsequently taken up by a second strain in which the product is formed. Moreover, this example shows that the product can be obtained in large excess from its precursor molecules.

### Example 4 Comparative Example—Synthesis of LST-c Using a One Strain Hybrid Process

In addition to the two-strain hybrid process described in example 2, this example describes the synthesis of LST-c using the one strain hybrid process to compare the performance of the two-strain system to a simpler one strain system.

In this one-strain hybrid process, the acceptor substrate LNnT was produced in situ from lactose by an LNnT strain with similar genotype as the LNnT strain in table 16, except the ptsG has not been deleted. Instead of using a second strain for the production of 6′-SL, as in example 2, a purified sialyl donor substrate 6′-SL was added during the cultivation. The trans-sialylation reaction was catalyzed by adding α-2,6-transsialidase from Photobacterium 85eiognathid JT-SHIZ-119 (PITS-197, SEQ ID NO: 88). The culture started with 700 g of mineral culture medium as described in the methods section above, containing lactose and 25 g/kg sucrose. The sucrose contained in the batch medium was depleted after approximately 15 h, after which a feed solution containing sucrose and minerals was fed continuously using a profile that kept the culture carbon limited, initially starting at a sucrose feed rate of 1.43 g/h and ramping up over 5 hours to 2.93 g/h whereafter it was kept constant. The temperature was initially set to 33° C. and was dropped to 30° C. with a linear 1 h ramp after 3 hours of feed. Sterile 6′SL was fed separately at a constant rate starting 15 hours into the fed-batch phase (corresponding to approximately 30 h after the inoculation/start of the fermentation) and lasting for 24 hours.

The transsialylation was started by adding 115 mg/L fermentation broth of α-2,6-transsialidase from Photobacterium 85eiognathid JT-SHIZ-119 (PITS-197, SEQ ID NO: 41) at 69.5 h after inoculation (start of the fermentation), when almost all the lactose had been converted into LNnT and the 6′SL had been added. Additional pulses of enzyme solution were added at 99 hours (366 mg/L), 121 hours (281 mg/L) and 146 hours (542 mg/L).

FIG. 7 shows the process progress curve in mass fraction of substrates and products relative to the total mass of substrates and products. As depicted, until the addition of the enzyme, lactose was nearly fully converted to LNnT by the E. coli strain. The increasing level of 6′-SL was due to the continued addition of the 6′-SL solution that lasted until the addition of the enzyme. From the point of enzyme addition at 69.5 h, the result of the enzymatic trans-sialylation can be seen in the formation of LST-c and depletion of 6′-SL. Moreover, the lactose concentration remained at a steady low level as the lactose side product released from the trans-sialylation reaction was quickly recycled by in vivo formation into LNnT. The performance of the one-strain hybrid production of LST-c was compared to the two-strain hybrid system and the in vitro synthesis of LST-c from 6′-SL and LNnT and the as described in example 2 and shown at FIGS. 4 and 5. Table 19 shows the comparison of the final product composition of LST-c synthesis using the different processes.

TABLE 19
Comparison of final product composition of the one-
strain hybrid process vs. the two-strain process
and the in vitro process for the synthesis of LST-c

| | Lactose | 6′-SL | LNnT | LST-c |
|---|---|---|---|---|
| Process | (wt. %) | (wt. %) | (wt. %) | (wt. %) |
| In vitro (ex 2) | 11.9 | 17.3 | 31.8 | 38.8 |
| Two strain Hybrid (ex. 2) | 5.8 | 20.0 | 19.5 | 54.6 |
| One-strain hybrid | 2.7 | 25.2 | 17.3 | 54.8 |

As can be seen in Table 19, the one-strain hybrid LST-c process is very similar to the two-strain hybrid process in terms of the amount of LST-c produced by the processes. This clearly indicates that the two-strain process is a very stable process and that the growth of two different strains in the same culture does not affect the ability to produce the desired product. The advantage of the two-strain process is that the second HMO does not have to be produced separately, purified, and then fed back into the fermentation, which makes the two-strain process cheaper and simpler than the one-strain process.

### Example 5 Synthesis of LST-a Using a Two-Strain Hybrid Process

This example describes the synthesis of LST-a using the two-strain hybrid process. The two strains, one producing LNT from lactose (substrate) and sucrose (carbon source) and the second producing 3′SL from lactose (substrate) and glucose (carbons source), were co-cultured. The 3′SL was produced as a sialyl donor substrate and the LNT as an acceptor substrate for a subsequent trans-sialyation reaction to LST-a catalyzed by a α-2,3-transsialidase (TcTS, SEQ ID NO: 113) added to the medium.

The synthesis of LST-a in the two-strain hybrid process was performed with the LNT producing strain (MF2, table 14, Example 1) and the 3′SL producing strain (MF1, table 14, Example 1). In the present example the strains were co-cultivated as described in Example 2, with the following changes. The culture started with 700 g of medium, lactose, 15 g/kg glucose and 15 g/kg sucrose. The co-culture was initiated with 1% (v/v) inoculum from each strain grown in pre-cultures with similar medium containing sucrose for the LNT strain and glucose for the 3′SL strain, both grown to an OD₆₀₀ of 2.5-5. After depletion of the glucose and sucrose contained in the batch medium after approximately 18 h, separate glucose and a sucrose mineral feed solutions were fed continuously at a rate of 1.17 g glucose/h and 1.17 g sucrose/h, which kept the co-culture carbon limited. The temperature was initially set to 33° C. and was dropped to 28° C. with a linear 1 h ramp at the start of the fed-batch phase. The trans-sialylation of LNT was started by the addition 339 mg/L fermentation broth of sterile filtered α-2,3-transsialidase (TcTS, SEQ ID NO: 113) 68 h after the inoculation/start of the fermentation and adding another pulse of the enzyme solution at 90 h.

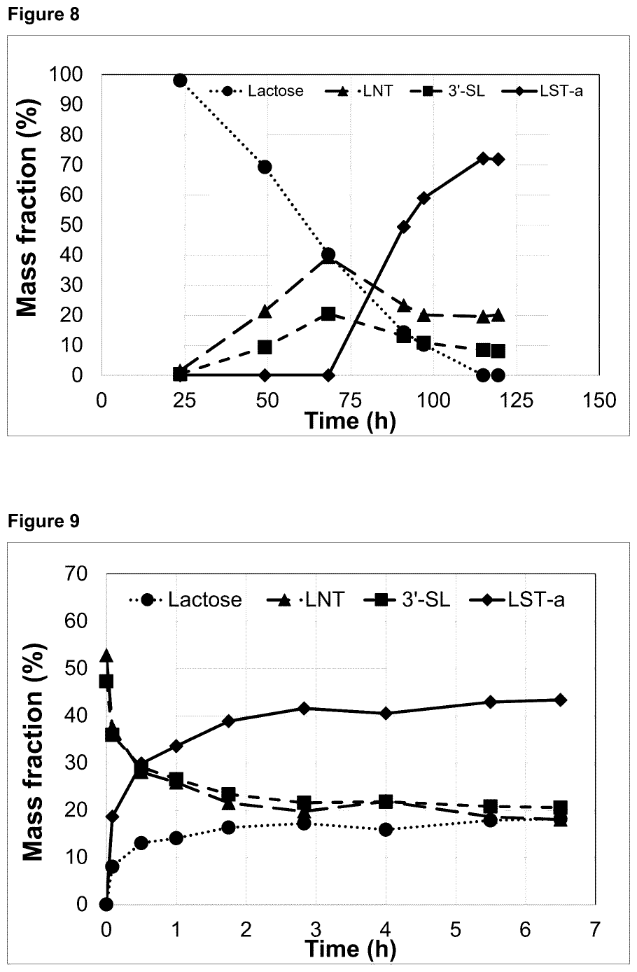

FIG. 8 shows the development of lactose, LNT, 3′SL and LST-a in weight % relative to the total weight of the substrates and products. The data until 68 h of the process (i.e., the addition of the enzyme) show the conversion of lactose to LNT and 3′SL by LNT strain and 3′SL strain, respectively, with no formation of LST-a. After the addition of the enzyme at 68 h, LNT and 3′SL decreased as increasing amounts of LST-a was formed. Moreover, even though lactose was released as a side product from the enzymatic reaction its concentration kept decreasing as it was rapidly recycled into LNT and 3′SL by the corresponding strains.

The two-strain hybrid process for the formation of LST-a was compared to the conventional in vitro enzymatic process for forming LST-a as described here.

The comparative in vitro experiment was performed using purified LNT and 3′SL as substrates. A substrate solution consisting of 150 mM LNT and 150 mM 3′SL was prepared at pH 6.5 and 25° C. The trans-sialylation reaction was started by adding 0.51 mg/mL α-2,3-transsialidase (TcTS, SEQ ID NO: 60). The progress of the reaction was monitored by measuring the concentrations of substrates and products by HPLC. Samples for the HPLC analysis were collected and immediately heat-treated at 90° C. for 5 min in order to quench the reaction. FIG. 9 shows the progress of the in vitro trans-sialylation of LNT using 3′SL as a sialyl donor for the synthesis of LST-a. In contrast to the hybrid process that led to a full conversion of 3′SL even when starting with two-fold higher (mol/mol) 3′SL level relative to lactose, the in vitro process only achieved 57% 3′SL conversion starting at 1:1 mol/mol of 3′SL/LNT. Table 20 shows a comparison of the final product composition of LST-a synthesis using the two-strain hybrid vs. in vitro processes.

TABLE 20
Comparison of final product composition of the two-strain hybrid
process vs. in vitro process for the synthesis of LST-a

| | Lactose | 3′SL | LNT | LST-a |
|---|---|---|---|---|
| Process | (wt. %) | (wt. %) | (wt. %) | (wt. %) |
| In vitro | 18.1 | 20.5 | 17.9 | 43.3 |
| Two-strain hybrid | 0 | 8.0 | 20.1 | 71.8 |

From table 20 it can be seen that the two-strain hybrid process for LST-a synthesis achieved full utilization of lactose and a higher LST-a fraction in contrast to the purely in vitro LST-a process as shown in table 20. Also the amount of 3′SL in the final mixture produced by the two-strain hybrid process is 2.5 times lower which offers an advantage in purification it is more challenging to separate 3′SL from LST-a than the neutral LNT.

### Example 6: Controlled Co-Fermentation of a 3′SL and a LNT Producing Strain with Different Carbon Source Ratios

In example 1 an LNT strain capable of growing on sucrose and with reduced ability to grow on glucose (MP2) was co-cultured with a 3′SL strain able to grow on glucose but not able to grow on sucrose (MF1). The strains were cultivated in a 1:1 ratio both in terms of carbon source in the batch phase and feed, and in terms of the inoculation ratio, i.e., how much of each strain that was added at the start of the cultivation.

This resulted in a 1:1 formation of LNT and 3′SL in the culture medium, as the two strains had similar product per carbon source yields (mole/g).

In the present example it was investigated whether a change in the ratio of the carbon sources, would lead to a mixture of LNT and 3′SL reflecting the change in the ratio.

Two fermentation examples were conducted using the fermentation procedure described in example 1, except for the changes in the amount of carbon source (batch phase and feed) and inoculation volume as indicated in table 21.

TABLE 21
Experimental setup with changes in carbon source and inoculation volume

| | | | | | Inoculation volume |
|---|---|---|---|---|---|
| | | | | Carbon source | relative to total |
| | | | | concentration in | inoculation volume |
| | | | | feed relative to | (inoculum volume |
| | | | | total carbon | relative to the |
| | | | Carbon source | source | initial culture |
| Experiment | Strain | Product | Batch phase | concentration. | volume) |
| Suc/Glc | MF2 | LNT | 66% sucrose (20 g) | 66% | 50% (1% (v/v)) |
| 66/33% | MF1 | 3′SL | 33% glucose (10 g) | 33% | 50% (1% (v/v)) |
| Suc/Glc | MF2 | LNT | 25% sucrose (7.5 g) | 25% | 25% (0.5% (v/v)) |
| 25/75% | MF1 | 3′SL | 75% glucose (22.5) | 75% | 75% (1.5% (v/v)) |

If the strains grow at a similar rate, the inoculation ratio would typically match the carbon source ratio to achieve a similar length of batch phase. However, in the Suc/Glc 66/73% experiment the inoculation ratio of the two strains was kept at 50/50% ratio as in example 1, to accommodate the fact that the MF2 strain still shows limited growth on glucose despite the ptsG deletion. If the inoculation ratio of the sucrose strain is higher than that of the glucose strain, the sucrose strain would be expected to consume a significant amount of the glucose present in the batch phase and skew the ratio of the strains off the intended 66/33 ratio. For this reason, the glucose strain (MF1), which does not grow on sucrose, was given a head start by inoculating it at a 50% ratio instead of the 33% of the glucose.

### Results

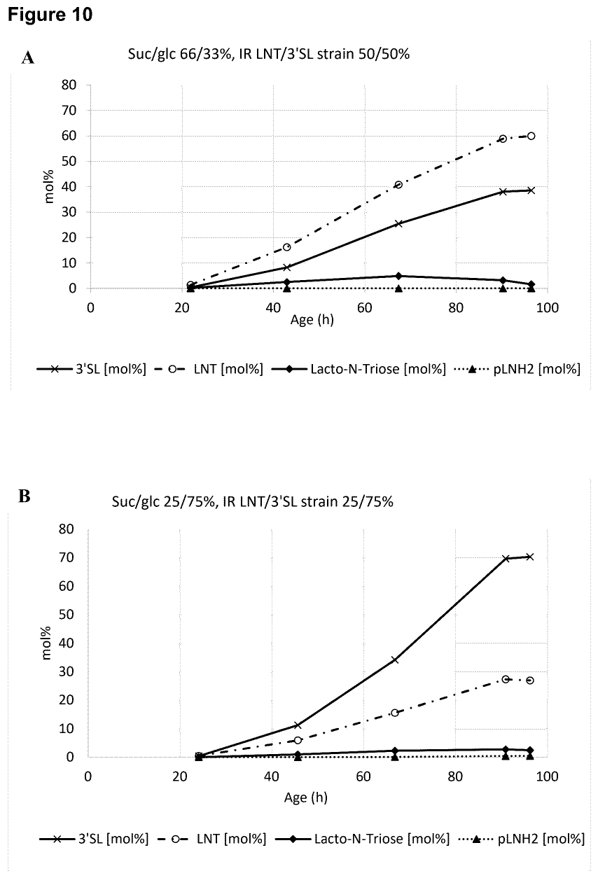

The results of the fermentation are shown in table 22 and 23 below and in FIGS. 10A and 10B as for the mole % of individual HMOs of the % of total HMO (mM)+lactose.

TABLE 22
Formation of HMO in a co-fermentation of a LNT strain (Suc) and 3′SL strain
(Glc) in a 66:33% ratio of Suc/Glc and inoculation ratio of LNT/3′SL of 50:50%

| Time | 3′SL | LNT | LNT-II | pLNH2 | 3′SL/(3′SL + LNT) | LNT/(3′SL + LNT) |
|---|---|---|---|---|---|---|
| (h) | [mol %] | [mol %] | [mol %] | [mol %] | [mol/mol %] | [mol/mol %] |
| 22 | 0.38 | 1.39 | 0.16 | 0 | 21.5 | 78.5 |
| 43 | 8.19 | 16.19 | 2.48 | 0 | 33.6 | 66.4 |
| 67 | 25.43 | 40.8 | 4.83 | 0 | 38.4 | 61.6 |
| 90 | 38 | 58.85 | 3.15 | 0 | 39.2 | 60.8 |
| 96 | 38.5 | 59.92 | 1.59 | 0 | 39.1 | 60.9 |

TABLE 23
Formation of HMO in a co-fermentation of a LNT strain (Suc) and 3′SL strain
(Glc) in a 25:75% ratio of Suc/Glc and inoculation ratio of LNT/3′SL of 25:75%

| Time | 3′SL | LNT | LNT-II | pLNH2 | 3′SL/(3′SL + LNT) | LNT/(3′SL + LNT) |
|---|---|---|---|---|---|---|
| (h) | [mol %] | [mol %] | [mol %] | [mol %] | [mol/mol %] | [mol/mol %] |
| 22 | 0.35 | 0.44 | 0 | 0 | 44.3 | 55.7 |
| 43 | 11.21 | 5.88 | 0.96 | 0 | 65.6 | 34.4 |
| 67 | 34.09 | 15.55 | 2.25 | 0.08 | 68.7 | 31.3 |
| 90 | 69.56 | 27.29 | 2.73 | 0.41 | 71.8 | 28.2 |
| 96 | 70.23 | 26.87 | 2.45 | 0.45 | 72.3 | 27.7 |

From table 22 it can be seen that at the end of the fermentation the ratio of LNT/3′SL was close to 61:39, which is quite close to the 66:33 carbon source ratio. This clearly indicates that the amount of LNT and 3′SL in the co-culture can be changed in a predictable manner by changing the ratio of the carbon sources and adapting the inoculation ratio to accommodate that the LNT strain still have limited growth on the 2^{nd} carbon source (Glc).

Table 23 clearly show that it is possible to invert amount of LNT and 3′SL produced in the co-culture by changing the carbon source ratio such that there is more glucose then sucrose in the fermentation leading to more 3′SL. In fact, the ratio of ends at 28:72, which is fairly close to the 25:75% ratio of the carbon sources used in the fermentation.

In combination with Example 1, this essentially shows that it is possible to produce a controlled mixture of HMOs from two strains in a single fermentation. This provides production flexibility in terms of strain design and fermentation scales, for example the need to express multiple glycosyltransferases in a single strain to produce a desired product mixture can be avoided.

### Example 7—Regeneration and Viability of Lyophilized Lactobacillus Species

Probiotics may be consumed as live bacteria or as a dried (e.g., lyophilized) product. Independent of the drying method, rehydration involves an important step in the recovery of dehydrated bacteria; an inadequate rehydration/regeneration step may lead to poor cell viability and a low final survival rate. Rehydration is therefore a highly critical step in the revitalization of a lyophilized culture. For both live and rehydrated bacteria, the survival of the bacteria under acidic conditions is critical since they need to pass through the acidic environment of the stomach and may also be faced with storage (shelf-life) in acidic food products.

In the present example it was tested whether the mixture of HMOs similar to the ones produced by the hybrid processes described in examples 2 and 5 can provide a benefit in the rehydration (regeneration) and viability of the probiotics. The test was performed under acidic conditions to resemble the conditions bacteria have to survive when passing through the stomach or when dosed in an acidic beverage.

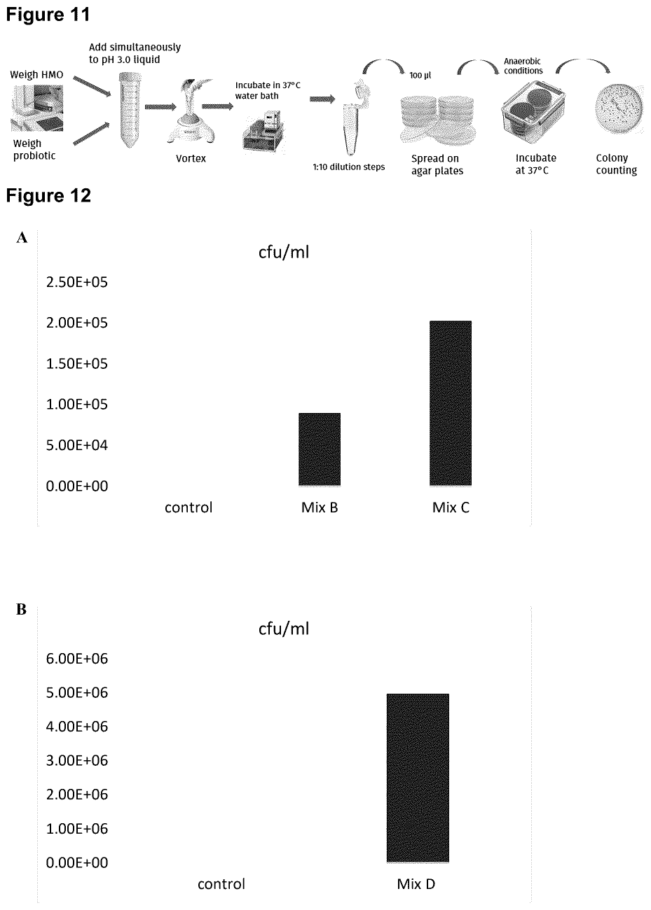

The lyophilized probiotic, Lactobacillus rhamnosus DSM 32550 (0.4 mg/ml), alone (control) or in combination with HMO mixtures (5% w/v) as indicated in table 24, were dissolved in sterile phosphate-buffered saline (PBS, pH=3), warmed to 37° C. and vigorously mixed for about 30 sec until no visible clumps remained. The tubes were incubated at 37° C. for 3 h. The samples were further diluted and 100 μl were spread in duplicates onto MRS agar plates which were incubated at 37° C. in anaerobic chambers. The regeneration and viability of the probiotics were determined by counting the colonies on the plates after 72 h of incubation. For the experimental setup, see FIG. 11.

TABLE 24
HMO compositions tested in the present example

| HMO | Control (A) | B | C | D |
|---|---|---|---|---|
| LST-a | 0 | 55 | 65 | 0 |
| LNT | 0 | 45 | 25 | 0 |
| 3′SL | 0 | 0 | 10 | 0 |
| LNnT | 0 | 0 | 0 | 25 |
| LST-c | 0 | 0 | 0 | 50 |
| 6′SL | 0 | 0 | 0 | 25 |

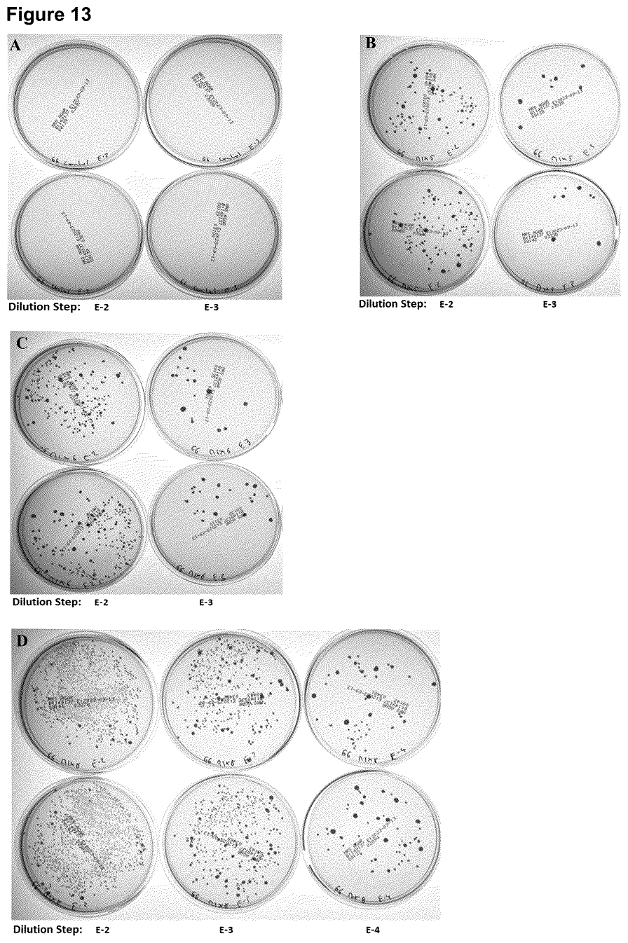

The CFU/ml was calculated based on colonies counted 72 hours after incubation (average of two plates). The E-2 dilution plates were used for counting mixtures B and C, results shown in FIG. 12A (LST-a containing) and E-4 dilution plates were used for counting mixture D, results shown in FIG. 12B. FIG. 13 shows picture of the plates with the colonies of Lactobacillus rhamnosus DSM 32550, the picture was taken after 6 days incubation to get bigger colonies.

Lyophilized Lactobacillus rhamnosus DSM 32550 dissolved with the HMO mixtures described herein showed an enhanced regeneration and survivability compared to control without the HMO mixtures, where survival was 0. These data clearly show that the regeneration and viability of Lactobacillus rhamnosus DSM 32550 after exposure to low pH conditions, such as in the stomach or in an acidic beverage, can be improved in the presence of any of the HMO mixtures. It can also be seen that a higher LST-a amount in combination with some 3′SL performs better than just the mixture of LST-a and LNT. mixture D (LST-c, LNnT and 6′SL) is more potent in terms of regeneration and viability of Lactobacillus rhamnosus DSM 32550 compared to mixture B and C.

To our knowledge it has not previously been shown that the tested mixtures provide a benefit of improving the regeneration and survivability of probiotics in an acidic environment.

### Example 8—Regeneration and Viability of Lyophilized Bifidobacterium Species

As in example 7 above the HMO mixtures in table 24 were also tested for their ability to provides a benefit of improving the regeneration and survivability of Bifidobacterium longum DSM 32946 in an acidic environment.

Lyophilized probiotic, Bifidobacterium longum DSM 32946 (0.4 mg/ml), alone or in combination with HMOs mixtures (5% w/v) as indicated in table 24, were dissolved into sterile pH 3.0 water, warmed to 37° C., and vigorously mixed for about 30 seconds until no visible clumps remained. The tubes were incubated at 37° C. for 30 minutes. Afterwards 100 μl were spread in duplicates onto MRS cysteine agar plates which were incubated for 48 h at 37° C. in anaerobic chambers. The regeneration and viability of the probiotics were determined by counting the colonies on the plates after 48 h of incubation.

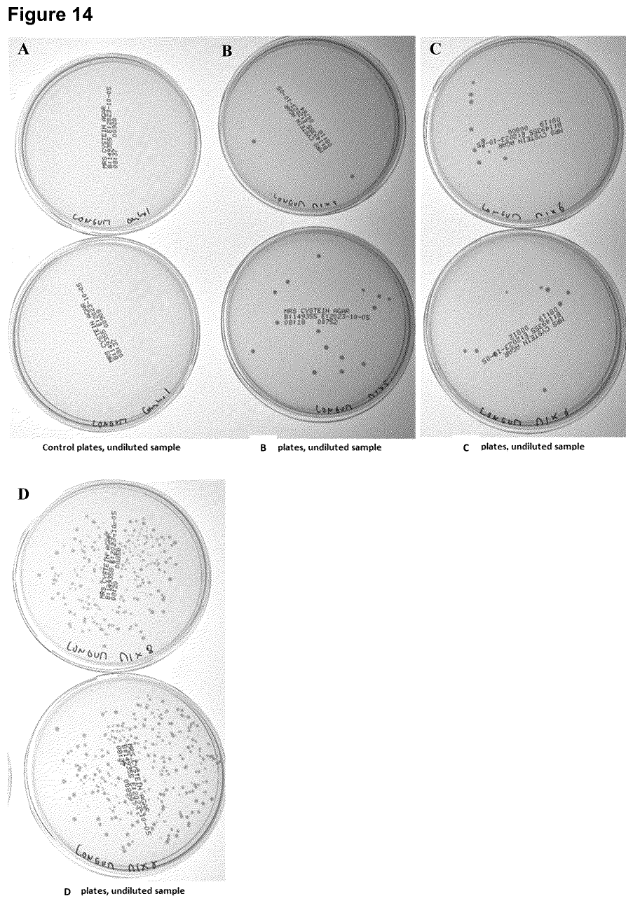

FIG. 14 shows picture of the plates with the colonies of Bifidobacterium longum DSM 32946 after 2 days incubation. The CFU/ml was calculated based on colonies counted on undiluted plates 48 hours after incubation (average of two plates). The results are shown in table 25.

TABLE 25
Average CFU/ml of Bifidobacterium longum DSM 32946 after
30 min acid treatment and 48 h subsequent incubation at 37° C.

| Mixture | Control (A) | Mix B | Mix C | Mix D |
|---|---|---|---|---|
| CFU/ml Average | 0 | 85 | 95 | 3245 |

As can be seen from table 25, the mixtures are capable making some Bifidobacterium longum DSM 32946 strains survive acid treatment compared to the control where the survival rate is 0.

### Example 9 Synthesis of Para-LNH Using a Two Strains Hybrid Process and a Trans-Lacto-N-Biosidase

In the present example it was illustrated that two strains (LNT-S2 and LNnT-S1) growing on different carbon sources (sorbitol and sucrose) could be co-cultured to produce a mixture of HMOs in the extracellular medium where they were converted in situ into a larger oligosaccharide (pLNH) by an enzymatic reaction.

In this example LNT functions as LNB donor substrate and LNnT as acceptor substrate for the in situ trans-lacto-N-biosidase reaction occurring in the fermentation broth. Specifically, the trans-lacto-N-biosidase transferred the non-reducing end of LNT (LNB, gal-β1,3-glcNAc) to the non-reducing end of LNnT to form the hexa-oligosaccharide, para-LNH and lactose (leaving group). The lactose was taken up by the LNT and LNnT strains and processed into more donor and acceptor substrates.

### Strains

The MDO strain described in the background strain section above was further engineered to generate a LNT producing strain and a LNnT producing strain with the genotypes shown in table 26.

TABLE 26
genotypes of strains used in the examples

| Strain ID | Product | Carbon Source | Genotype |
|---|---|---|---|
| MDO | None | +sorbitol | F-λ- ΔendA1 ΔrecA1 ΔrelA1 ΔgyrA96 Δthi-1 glnV44 |
| | | ↓Sucrose | hsdR17(rK-mK-) ΔlacZ wcaF::Plac ΔnanKETA ΔlacA |
| | | | ΔmelA ΔwcaJ ΔmdoH |
| LNnT-S1 | LNnT | +sucrose | MDO 2x PglpF-IgtA⁴, 2x-PglpF-galT¹, 2xPglpF-Edic1², |
| | | ↓sorbitol | Δlacl³, scrYA⁶ Pscr-scrBR⁶, PglpF-lacY⁸, ΔsrlA⁹, ΔglpR¹⁰ |
| LNT-S2 | LNT | +sorbitol | MDO + 3x PglpF-IgtA⁴ 2x PglpF-galTK⁵, PglpF-nec⁷ |
| | | ↓sucrose | PglpF-lacY⁸ Δlacl³, ΔglpR¹⁰ |

¹galT - one genomically inserted gene encoding β-1,4-Galactosyltransferase (SEQ ID NO: 105) under control of a PglpF promoter (SEQ ID NO: 93).
²Edic1 - genomically integrated MFS transporter (SEQ ID NO: 124) under control of the PglpF promoter (SEQ ID NO_93)
³Δlacl - Deletion of lac repressor makes use of IPTG obsolete.
⁴lgtA -genomically inserted copies of a gene encoding β-1,3-N-acetyl-glucosaminyltransferase (SEQ ID NO: 94) under control of a PglpF promoter (SEQ ID NO: 93).
⁵galTK - two genomically inserted gene encoding β-1,3-Galactosyltransferase (SEQ ID NO: 96) under control of a PglpF promoter (SEQ ID NO: 93).
⁶scrYA, scrBR two operons encoding the sequences of SEQ ID NO: 97 and 98 and SEQ ID NO: 99 and 100 respectively, and under the control of the PglpF_SD1 promoter (SEQ ID NO: 101) and Pscr promoter (SEQ ID NO: 102), respectively.
⁷nec gene coding for a heterologous major facilitator superfamily (MFS) transporter of SEQ ID NO: 107 under control of a PglpF promoter (SEQ ID NO: 93).
⁸lacY - additional genomically integrated copy of lacY (SEQ ID NO: 1) under control of a PglpF promoter (SEQ ID NO: 93).
⁹ΔsrlA - Deletion of the sorbitol PTS complex IICB^{slr} - to limit growth on sorbitol.
¹⁰ΔglpR - Deletion of glpR repressor

### Fermentation

The 2-strain hybrid process was carried out in a in 250 mL fermenters (Ambr 250 Bioreactor system, Sartorius) starting with 100 mL of mineral culture medium containing 15 g/kg sorbitol (sterilized separately) and 15 g/kg sucrose (sterilized separately), 40 g/kg lactose monohydrate (sterilized separately), (NH₄)₂HPO4, KH₂PO₄, MgSO₄×7H₂O (sterilized separately), KOH, NaOH, citric acid, trace element solution, antifoam and thiamine (filter sterilized). The dissolved oxygen level was kept at 20% by a cascade of first agitation starting at 300 rpm (up to max 3000 rpm) and then airflow of 1 VVM (up to max 3 VVM). The pH was kept at 6.8 by titration with NH₄OH solution. Precultures were grown in shake-flasks on either glucose (LNT-S2, sorbitol strain) or sucrose (LNnT-S1, sucrose strain) in a similar medium. The cultivation in the bioreactor was started with a total inoculum of 2% (v/v). Since the LNnT-S1 strains growth rate on sucrose was significantly faster than the LNT-S2 strains on sorbitol, the LNT strain was inoculated at a 20:1 ratio compared to the LNnT to try to achieve an equal batch length.

The initiation of the feeding-phase was triggered by an increase in pH (>6.85), indicating depletion of the sorbitol and sucrose contained in the batch medium. A feed solution containing sorbitol and sucrose at a 1:1 ratio (w/w) (sterilized together separately from the minerals), MgSO₄×7H₂O, H₃PO₄, trace metals and antifoam, was fed continuously using a constant profile that kept the culture carbon limited. The temperature was initially set to 34° C. and was dropped to 28° C. with a linear 1 h ramp after 3 hours of feed.

The growth and metabolic activity and state of the cells were followed by on-line measurements of agitation, ammonium hydroxide base addition, temperature, reflectance, pH, respiratory quotient and CO₂ evolution rate.

The trans-lacto-N-biosidase reaction was started by adding 0.2 mL of 100 mg/mL stock solution of lacto-N-biosidase from Bifidobacterium longum (LNbX, SEQ ID NO: 122) at 48 hours and 96 hours of fermentation time. Lactose and oligosaccharide concentrations were determined via HPLC on samples collected during the fermentation. Samples for HPLC analysis were heat treated at 90° C. for 20 min at the time of collection to stop the enzymatic process.

### Results

Table 27 shows concentration data illustrating the process progress in weight % relative to the total weight of substrates and products involved in the trans-lacto-N-biosidase reaction occurring in the culture medium. In the HPLC method used to analyze the samples it was not possible to differentiate LNT and LNnT, since they elute at the same time point.

TABLE 27
Weight % relative to the total weight of substrates, products
and by-products as measured throughout the fermentation.

| Fermentation time | Lactose × H₂O | LNT + LNnT | pLNH | LNB |
|---|---|---|---|---|
| Age [h] | 66.06 | 33.66 | 0.28 | 0.00 |
| 50 | 66.06 | 33.66 | 0.28 | 0.00 |
| 73 | 33.17 | 61.73 | 3.33 | 1.76 |
| 92 | 13.36 | 77.69 | 4.39 | 4.56 |
| 116 | 2.58 | 84.04 | 6.12 | 7.27 |

From table 27 it can be seen that it is possible to produce pLNH as a product in the two-strain hybrid fermentation process.

The disaccharide LNB, is most likely produced from a hydrolytic side activity of the trans-lacto-N-biosidase which most likely is able to cleave LNT into lactose and LNB.

As indicated in the fermentation section above the growth rates of the LNT-S2 and LNnT-S1 strains is quite different, and the strains were therefore pitched in a 20:1 ratio. Despite the difference in pitching rates the sorbitol strain producing LNT was very slow in consuming all the sorbitol and therefore the production of LNT probably was quite low for the first 50-60 hours of the fermentation. Therefore, the amount of LNT+LNnT converted at the end of the fermentation was lower than expected (84% is still left in the broth), but still a significant amount of pLNH was produced showing that it was possible to produce pLNH in a combined fermentative and enzymatic process. To our knowledge this is the first time pLNH has been produced in a fermentation process, not requiring initial fermentation and purification of the donor and acceptor substrates.

The concentration of the substrates and products shown in table 27 can be adjusted by optimizing the process, for example by tweaking the inoculation ratio of the two strains or giving the LNT-S2 strain a head start, and potentially also tweaking the sucrose sorbitol ratio in the fermentation with the respective yields of the two strains to generate sufficient amount of LNT while maintaining excess amounts of LNnT to LNT (preferably a ratio of 1:5 of LNT:LNnT).

### Example 10 Synthesis of Sialyl-Lewis X Using a Two Strains Hybrid Process and a Transsialidase

In the present example it was illustrated that two strains (S-LacNAc and Lewis X) growing on different carbon sources (sorbitol and sucrose, respectively) could be co-cultured with LacNAc as a substrate to produce a mixture of non-HMO oligosaccharides in the extracellular medium where the oligosaccharides were reacted with a transsialidase catalyzing the production of a larger sialylated oligosaccharide in the medium of the fermentation (in situ).

In this example sialyl-LacNAc functioned as sialyl donor substrate and Lewis X as acceptor substrate for the in situ transsialyation reaction occurring in the fermentation broth. Specifically, the transsialidase transferred the sialyl group in an α-2,3 linkage to the galactose moiety of the Lewis X oligosaccharide producing sialyl-Lewis X. Following the formation of sialyl-Lewis X, LacNAc was released (leaving group) and re-cycled by the S-LacNAc and Lewis X strains and processed into more donor and acceptor substrates.

The fermentation with S-LacNAc and Lewis X strains (genotypes in table 28) was conducted as described in example 9 (inoculation ratio 20:1 of Lewis X: S-LacNAc) with the difference that LacNAc was used as initial substrate instead of lactose and the enzyme LNbX was substituted with α-2,3-transsialidase (TcTS, SEQ ID NO: 113), but supplied in the same amount.

### Strains

The MDO strain described in the background strain section above was further engineered to generate a S-LacNAc producing strain and a Lewis X producing strain with the genotypes shown in table 28.

TABLE 28
genotypes of strains used in the example

| | | Carbon | |
|---|---|---|---|
| Strain ID | Product | Source | Genotype |
| MDO | None | +sorbitol | F-λ- ΔendA1 ΔrecA1 ΔrelA1 ΔgyrA96 Δthi-1 glnV44 |
| | | ↓Sucrose | hsdR17(rK-mK-) ΔlacZ wcaF::Plac ΔnanKETA ΔlacA |
| | | | ΔmelA ΔwcaJ ΔmdoH |
| S-LacNAc | sialyl- | +sucrose | MDO: PglpF-Poral¹, Pscr-scrYA², Pscr-scrBR², PglpF-nec³, |
| | LacNAc | ↓sorbitol | PglpF-neuA⁴, PglpF-neuB⁵, PglpF-neuC⁶, ΔsrlA⁷ |
| Lewis X | Lewis X | +sorbitol | MDO: PglpF-CA⁸, 2xPglpF-futA⁹, PglpF-marc¹⁰, Δlacl¹¹ |
| | | ↓sucrose | |

¹poral - one genomic copy of a gene encoding the α-2,3-sialyltransferase of SEQ ID NO: 125 under control of a PglpF promoter (SEQ ID NO: 93).
²scrYA, scrBR two operons encoding the sequences of SEQ ID NO: 97 and 88 and SEQ ID NO: 99 and 100 respectively, and under the control of the Pscr promoter (SEQ ID NO: 102).
³nec gene coding for a heterologous major facilitator superfamily (MFS) transporter of SEQ ID NO: 107 under control of a PglpF promoter (SEQ ID NO: 93).
⁴neuA one genomic copy of a gene encoding the CMP-Neu5Ac synthetase from Campylobacter jejuni (GenBank AAK91728.1)
⁵neuB one genomic copy of a gene encoding the Neu5Ac synthase from Campylobacter jejuni (GenBank AAK91726.1)
⁶neuC one genomic copy of a gene encoding the UDP-GlcNAc 2-epimerase from Campylobacter jejuni (GenBank AAK91727.1)
⁷ΔsrlA - Deletion of the sorbitol PTS complex IICB^{slr} - to limit growth on sorbitol
⁸CA = extra colanic acid gene cluster (gmd-wcaG-wcaH-wcal-manC-manB, SEQ ID NO: 109) under the control of a PglpF promoter at a locus that is different than the native locus.
⁹futA - three independent genomic copies of the gene encoding α-1,3-fucosyl-transferase of SEQ ID NO: 90 under control of the PglpF promoter (SEQ ID NO: 93).
¹⁰marc gene coding for a heterologous major facilitator superfamily (MFS) transporter of SEQ ID NO: 90.
¹¹Δlacl - Deletion of lac repressor makes use of IPTG obsolete.

### Results

Table 29 shows concentration data illustrating the process progress in weight % relative to the total weight of substrates and products involved in the transsialidase reaction occurring in the culture medium.

TABLE 29
Weight % relative to the total weight of substrates
and products as measured throughout the fermentation.

| Fermentation | | | | |
|---|---|---|---|---|
| time | LacNAc | LewisX | Sialyl-LacNAc | Sialyl-Lewis X |
| [h] | wt % | wt % | wt % | wt % |
| 50 | 24.2 | 0.0 | 32.9 | 0.0 |
| 73 | 11.0 | 0.0 | 72.4 | 0.0 |
| 92 | 1.2 | 6.1 | 88.1 | 2.7 |
| 116 | 3.8 | 5.5 | 71.5 | 13.4 |

From table 29 it can be seen that the 20:1 inoculation ratio of the two strains was not enough to offset the difference in growth rates on sorbitol vs sucrose causing an imbalance in the donor and acceptor with much higher sialyl-LacNAc (sucrose strain) produced compared to Lewis X (sorbitol strain). The sucrose growing sialyl-LacNAc strain finished the batch growth and triggered the fed-batch phase after 24 hours, whereas the sorbitol growing Lewis X strain first consumed all the accumulated sorbitol after 85 hours, whereafter it became carbon limited. Since limited amounts of Lewis X was produced in the first 85 hours the enzymatic reaction in the culture broth only started after 85 hours. Once the Lewis X strain started to produce Lewis X, Sialyl-Lewis X quickly accumulated from the enzymatic reaction. Since very little LacNAc accumulated compared to the produced sialyl-Lewis X it showed that it was recycled to produce more sialyl-LacNAc and Lewis X.

If more sialyl-Lewis X is desired it is possible to optimize the process, for example by tweaking the inoculation ratio of the two strains even further, or giving the Lewis X strain a head start, and potentially also tweaking the sucrose:sorbitol ratio in the fermentation with the respective yields of the two strains to generate equal amounts of Lewis X and sialyl-LacNAc. Furthermore, the process can be designed to full conversion of the charged sialyl-LacNAc by terminating the sucrose feed used by the S-LacNAc strain before the end of the entire process. Thereby, the purification of the charged sialyl-Lewis X is easier since the only remaining product would be neutral Lewis X.

### Example 11: Production of Sialyl-LacNAc Using a Two-Strain System with Intermediate Disaccharide Uptake

In the present example a LacNAc strain (strain 1) capable of growing on sorbitol was co-cultured with a strain expressing an alpha-2,3-sialyltransferase growing on sucrose (2,3-ST strain/strain 2). This process corresponded to the process illustrated in FIG. 1, except that the first strain produced the precursor disaccharide (LacNAc) directly from the 1^{st} carbon source (sorbitol) without taking up any additional initial substrate such as lactose depicted in FIG. 1. The LacNAc (precursor disaccharide) was then taken up by second strain which expressed a sialyltransferase decorating the 3-positon of the galactose moiety in LacNAc to produce the non-HMO oligosaccharide sialyl-LacNAc (3′S-LacNAc).

The co-fermentation with the LacNAc and the 2,3-ST strains (genotypes in table 30) was conducted as described in example 9 (inoculation ratio 20:1 of LacNAc:2,3-ST), with the difference that no initial substrate (lactose) was added to the fermentation and no enzyme was added to the fermentation.

### Strains

The MDO strain described in the background strain section above was further engineered to generate a LacNAc producing strain and a strain expressing an alpha-2,3-sialyltransferase (2,3-ST strain) with the genotypes shown in table 30. The efficient engineering of a strain capable of overproducing GlcNAc, which is need in the LacNAc producing strain is further described in LU et al 2022 Biotechnology Notes vol 3 p 15-25.

TABLE 30
genotypes of strains used in the example

| | | Carbon | |
|---|---|---|---|
| Strain ID | Product | Source | Genotype |
| MDO | None | +sorbitol | F-λ- ΔendA1 ΔrecA1 ΔrelA1 ΔgyrA96 Δthi-1 glnV44 |
| | | ↓Sucrose | hsdR17(rK-mK-) ΔlacZ wcaF::Plac ΔnanKETA ΔlacA |
| | | | ΔmelA ΔwcaJ ΔmdoH |
| Strain 1 | LacNAc | +sorbitol | MDO: 1x PglpF-GNA1-⁸, 1x-PglpF-GlnA⁹, 1x-PglpF-galT¹⁰, |
| LacNAc | | ↓sucrose | 1xPglpF-yqaB¹¹, 1xPglpF-glmS*54¹², ΔnagABE, ΔnagK, |
| | | | ΔmurQ, ΔmanXYZ |
| Strain 2 | sialyl- | +sucrose | MDO: PglpF-Poral¹, Pscr-scrYA², Pscr-scrBR², PglpF-nec³, |
| 2,3-ST | LacNAc | ↓sorbitol | PglpF-neuA⁴, PglpF-neuB⁵, PglpF-neuC⁶, ΔsrlA⁷ |

¹poral - one genomic copy of a gene encoding the α-2,3-sialyltransferase of SEQ ID NO: 125 under control of a PglpF promoter (SEQ ID NO: 93).
²scrYA, scrBR two operons encoding the sequences of SEQ ID NO: 97 and 88 and SEQ ID NO: 99 and 100 respectively, and under the control of the Pscr promoter (SEQ ID NO: 102),
³nec gene coding for a heterologous major facilitator superfamily (MFS) transporter of SEQ ID NO: 107 under control of a PglpF promoter (SEQ ID NO: 93).
⁴neuA one genomic copy of a gene encoding the CMP-Neu5Ac synthetase from Campylobacter jejuni (GenBank AAK91728.1)
⁵neuB one genomic copy of a gene encoding the Neu5Ac synthase from Campylobacter jejuni (GenBank AAK91726.1)
⁶neuC one genomic copy of a gene encoding the UDP-GlcNAc 2-epimerase from Campylobacter jejuni (GenBank AAK91727.1)
⁷ΔsrlA - Deletion of the sorbitol PTS complex IICB^{slr} - to limit growth on sorbitol
⁸GNA1 - one genomic copy of a gene encoding glucosamine-phosphate N-acetyltransferase 1 (GenBank NP_116637.1) under control of a PglpF promoter (SEQ ID NO: 93).
⁹GlnA - one genomic copy of a gene encoding Glutamine synthetase1 (GenBank WP_001271717.1) under control of a PglpF promoter (SEQ ID NO: 93).
¹⁰galT - one genomically inserted gene encoding β-1,4-Galactosyltransferase (SEQ ID NO: 105) under control of a PglpF promoter (SEQ ID NO: 93).
¹¹yqaB - one genomic copy of a gene encoding Fructose-1-phosphate phosphatase (GenBank WP_000273290.1) under control of a PglpF promoter (SEQ ID NO: 93).
¹²glmS*54 - Glucosamine synthase variant with a triple mutation (A38T, R249C, G471S, Deng et al. 2006 Biochimie vol 88 p. 419-429) that effectively reduces the inhibitory effect of the product formed from the glmS-mediated reaction, namely GlcN-6P.
¹³ΔnagABE - deletion of gene cluster that prevents conversion of GlcNAc-6 phosphate to fructose-6-phosphate.
¹⁴ΔnagK - deletion of gene encoding N-acetyl-D-glucosamine kinase preventing conversion of GlcNAc to GlcNAc-6 phosphate.
¹⁵ΔmurQ - deletion that prevents depleting GlcNAc-6 phosphate.
¹⁶ΔmanXYZ - deletion that blocks GlcNAc in and out of the cell.

### Results

Table 31 shows concentration data illustrating the process progress in weight % relative to the total weight of substrates and products.

TABLE 31
Weight % relative to the total weight of substrates
and products as measured throughout the fermentation.

| Fermentation time | LacNAc | Sialyl-LacNAc |
|---|---|---|
| [h] | wt % | wt % |
| 50 | 32.5 | 67.5 |
| 73 | 15.5 | 84.5 |
| 92 | 10.2 | 89.8 |
| 116 | 8.5 | 91.5 |

In this example the inoculation ratio was well suited to the growth rates of the strains, since the LacNAc strain growing on sorbitol was capable of producing sufficient LacNAc which was taken up by the 2,3-ST strain where the LacNAc was sialylated to produce sialyl-LacNAc with >90% purity.

### Example 12: Production of Lewis X Using a Two-Strain System with Intermediate Disaccharide Uptake

In the present example a LacNAc strain (strain 1) capable of growing on sorbitol was co-cultured with a strain expressing an alpha-1,3-fucosyltransferase growing on sucrose (1,3-FT strain/strain 2). This process corresponds to the process illustrated in FIG. 1, except that the first strain produced the precursor disaccharide (LacNAc) directly from the 1^{st} carbon source (sorbitol) without taking up an additional initial substrate such as lactose as depicted in FIG. 1. When produced, the LacNAc (precursor disaccharide) was taken up by second strain which expressed a fucosyltransferase decorating the 3-positon of the glcNAc moiety in LacNAc to produce the non-HMO oligosaccharide Lewis X.

The co-fermentation with the LacNAc and the 1,3-FT strains (genotypes in table 32) was conducted as described in example 9 (inoculation ratio 20:1 of LacNAc:1,3-FT), with the difference that no initial substrate (lactose) was added to the fermentation and no enzyme was added to the fermentation.

### Strains

Th MDO strain described in the background strain section above was further engineered to generate a LacNAc producing strain and a strain expressing an alpha-1,3-fucosyltransferase (1,3-FT strain) with the genotypes shown in table 32.

TABLE 32
genotypes of strains used in the example

| | | Carbon | |
|---|---|---|---|
| Strain ID | Product | Source | Genotype |
| MDO | None | +sorbitol | F-λ- ΔendA1 ΔrecA1 ΔrelA1 ΔgyrA96 Δthi-1 glnV44 |
| | | ↓Sucrose | hsdR17(rK-mK-) ΔlacZ wcaF::Plac ΔnanKETA ΔlacA |
| | | | ΔmelA ΔwcaJ ΔmdoH |
| Strain 1 | LacNAc | +sorbitol | See strain 1 in table 30 |
| LacNAc | | ↓sucrose | |
| Strain 2 | Lewis X | +sucrose | MDO: PglpF-CA¹, PglpF_SD1-Pscr-scrYA², Pscr-scrBR², |
| 1,3-FT | | ↓sorbitol | 2xPglpF-futA³, PglpF-marc⁴, Δlacl⁵, ΔsrlA⁶ |

¹CA = extra colanic acid gene cluster (gmd-wcaG-wcaH-wcal-manC-manB, SEQ ID NO: 109) under the control of a PglpF promoter at a locus that is different than the native locus.
²scrYA, scrBR two operons encoding the sequences of SEQ ID NO: 97 and 88 and SEQ ID NO: 99 and 100 respectively, and under the control of the PglpF_SD1 promoter (SEQ ID NO: 101) and Pscr promoter (SEQ ID NO: 102), respectively,
³futA - three independent genomic copies of the gene encoding α-1,3-fucosyl-transferase of SEQ ID NO: 90 under control of the PglpF promoter (SEQ ID NO: 93).
⁴marc gene coding for a heterologous major facilitator superfamily (MFS) transporter of SEQ ID NO: 90.
⁵Δlacl - Deletion of lac repressor makes use of IPTG obsolete.
⁶ΔsrlA - Deletion of the sorbitol PTS complex IICB^{slr} - to limit growth on sorbitol.

### Results

Table 33 shows concentration data illustrating the process progress in weight % relative to the total weight of substrates and products.

TABLE 33
Weight % relative to the total weight of substrates
and products as measured throughout the fermentation.

| Fermentation time | LacNAc | Lewis X |
|---|---|---|
| [h] | wt % | wt % |
| 50 | 40.9 | 59.1 |
| 73 | 33.8 | 66.2 |
| 92 | 12.7 | 87.3 |
| 116 | 6.8 | 93.2 |

In this example the inoculation ratio was well suited to the growth rates of the strains, since the LacNAc strain growing on sorbitol was capable of producing sufficient LacNAc which was taken up by the 1,3-FT strain where the LacNAc was fucosylated to produce Lewis X with >90% purity.

### Example 13 In Vivo Production of Transsialidase Enzymes for the Hybrid Process Producing LST-a or LST-c

The present example sets out to illustrate that the transglycosidases which have been sterile filtered into the fermentations described in examples 2 and 5 above could be expressed in vivo in a strain producing one of the donor or acceptor oligosaccharides and when a second oligosaccharide is provided to the culture medium of the strain producing the transglycosidase a third more complex oligosaccharide was formed.

In the example a number of signal peptides were screened for their ability to facilitate the production and export of the transglycosidase in an oligosaccharide producing strain.

The screening was conducted in a deep well format, which for practical reasons are not suitable for growing two strains on different carbon sources since the feeding and monitoring of the strains is limited in such an assay. Therefore, in the present assay the second oligosaccharide was added to the deep well to illustrate that together with the first oligosaccharide and the transglycosidase produced by the strain the desired more complex oligosaccharide was produced in the culture.

Specifically, screening setups to produce either LST-a (1) or LST-c (2) were performed as listed in table 35.

TABLE 35
screening of heterologous transglycosidase expression in E. coli

| | Donor HMO | | Transglycosidase | | |
|---|---|---|---|---|---|
| | strain | Genotype* | expressed | Acceptor | Product |
| 1 | 3′SL | MDO: PglpF-Poral, Pscr-scrYA, | α-2,3-transsialidase | LNT | LST-a |
| | | Pscr-scrBR, PglpF-nec³, PglpF- | (TcTS, SEQ ID NO: | | |
| | | neuA, PglpF-neuB, PglpF-neuC | 113) | | |
| 2 | 6′SL | MDO: PglpF-ST6, Pd2, Pscr-scrYA, | α-2,6-transsialidase | LNnT | LST-c |
| | | Pscr-scrBR, PglpF-nec, PglpF-neuA, | (PITS-197, SEQ ID | | |
| | | PglpF-neuB, PglpF-neuC | NO: 88) | | |

*the elements in the genotype of the 3′SL strain can be found in table 28 and for the 6′SL strain in table 16 and 28, and the MDO background strain is described in the background strain section.

For the screening each strain was transformed with a pMK plasmid containing pBR322 as origin of replication and kanamycin as selection marker. The plasmid further contained the transglycosidase with a test signal peptide (selected from table 34) at the N-terminal end. The signal peptide-transglycosidase construct was under control of the PmgIB_70UTR_SD4 promoter (SEQ ID NO: 126).

### Deep Well Assay

The strains used in the following examples were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium supplemented with magnesium sulphate, thiamine, glucose, and kanamycin for plasmid preservation. The precultures were incubated for 24 hours at 34° C. and 1000 rpm shaking and then further transferred to a new basal minimal medium (BMM, pH 7,5) in order to start the main culture. The new BMM was supplemented with magnesium sulphate, thiamine, a bolus of 20% glucose solution (0.1-0.15 g/L) and a bolus of 20% lactose solution (16 g/L). Moreover, 20% maltodextrin (18 g/L) was provided as carbon source, accompanied by the addition of glucoamylase DSM (A. niger), such that glucose was released at a rate suitable for carbon limited growth. Kanamycin was also added to the main culture media (final concentration in the media 50 μg/ml). The acceptor HMO, either LNT (setup 1) or LNnT (setup 2), respectively for LST-a or LST-c production, were also added to the main culture media (a bolus of 20% HMO, 16 g/L). The main cultures were incubated for 96 hours at 28° C. and 1000 rpm shaking.

At the end of the assay samples were boiled for 1 h. This was followed by centrifugation at 4700 rpm for 10 minutes, where after the resulting supernatant was diluted 5× in acetonitrile and analysed by HPLC.

### Results

In screening setup 1, a strain with an alpha-2,3-sialyltransferase and a plasmid for expression of the α-2,3-transialidase (TcTs) was cultivated in the presence of lactose and LNT was added to the culture to investigate if the cell was capable of producing 3′SL and the α-2,3-transsialidase (TcTs) in the culture medium and transfer the sialyl group from the donor 3′SL to the acceptor LNT. The plasmids were constructed with different signal peptides to identify the best sequence to produce the α-2,3-transialidase (TcTs) extracellularly. A strain capable of producing 3′SL but without the plasmid was used as control. The results for the selected signal peptides are shown in table 36 as % of LST-a of the total amount of LST-a and the 3′SL produced by the cell.

TABLE 36
relative amount of LST-a produced in screening setup 1

| Signal peptide | Relative LST-a | Relative 3′SL |
|---|---|---|
| Control (no plasmid) | 0.5 | 99.5 |
| STII | 19.9 | 80.1 |
| LivK | 34.2 | 65.8 |
| FhuD | 31.2 | 68.8 |
| MdoD | 34.2 | 65.8 |
| TorT | 35.7 | 64.3 |

From table 36 it can be seen that the most suitable signal peptides resulted in the range of 20 to 36% of LST-a of the total amount of LST-a and 3′SL (table 36). This clearly indicated that the transsialidase was produced together with 3′SL and was capable of performing the transsialyation reaction in the culture medium when the acceptor substrate LNT was added to the culture. In contrast less than 1% of LST-a was produced in the control cell not expressing any transsialidase.

In screening setup 2, a strain with an alpha-2,6-sialyltransferase and a plasmid for expression the α-2,6-transsialidase (PITS-197) was cultivated in the presence of lactose and LNnT was added to the culture to investigate is the cell was capable of producing 6′SL and the α-2,6-transsialidase (PITS-197 in the culture medium and transfer the sialyl group from the donor 6′SL to the acceptor LNnT. The plasmids were constructed with different signal peptides in order to find the best sequence to produce the α-2,6-transialidase (PITS-197) extracellularly. A strain capable of producing 6′SL, but without the plasmid was used as control. The results for selected signal peptides are shown in table 37 as % of LST-c of the total amount of LST-c and the 6′SL produced by the cell.

TABLE 37
relative amount of LST-c produced in screening setup 2

| Signal peptide | Relative LST-c | Relative 6′SL |
|---|---|---|
| Control (no plasmid) | 0.4 | 99.6 |
| OmpC | 20.2 | 79.8 |
| OmpT | 17.0 | 83.0 |
| STII | 18.0 | 82.0 |

From table 37 it can be seen that the most suitable signal peptides resulted in the range of 17 to 20% of LST-c of the total amount of LST-c and 6′SL (table 36). This clearly indicates that the transsialidase was produced together with 6′SL and was capable of performing the transsialyation reaction in the culture medium when the acceptor substrate LNnT was added to the culture. In contrast less than 0.5% of LST-c was produced in the control cell not expressing any transsialidase.

### Example 14 In Vivo Production of the Transfucosidase Enzyme for the Hybrid Process Producing LNFP-III

In the present example it was illustrated that two strains growing on different carbon sources can be co-cultured where one strain produces an acceptor molecule, and the other strain produces a donor molecule. In addition, the transglycosidase enzyme needed to facilitate the formation of the third more complex HMO, by transferring a glycosidase moiety from the donor to the acceptor molecule, was produced by one of the cells and exported into the fermentation broth.

In this example, a sucrose growing strain producing LNnT from lactose (LNnT-S2) was co-cultured with a sorbitol growing strain producing 3FL from lactose (3FL-S1). Both HMOs were exported into the medium. In addition, the 3FL-S1 strain was also producing and exporting a transfucosidase enzyme into the medium capable of transferring the fucose moiety from 3FL onto the LNnT molecule to form LNFP-III and lactose in the culture medium. The lactose released from the enzymatic reaction was reused by the strains in the culture to form more 3FL and LNnT.

### Strains

The MDO strain described in the background strain section above was further engineered to generate a LNT producing strain and a LNnT producing strain with the genotypes shown in table 38.

TABLE 38
genotypes of strains used in the examples

| | | Carbon | |
|---|---|---|---|
| Strain ID | Product | Source | Genotype |
| MDO | None | +sorbitol | F-λ- ΔendA1 ΔrecA1 ΔrelA1 ΔgyrA96 Δthi-1 glnV44 |
| | | ↓Sucrose | hsdR17(rK-mK-) ΔlacZ wcaF::Plac ΔnanKETA ΔlacA |
| | | | ΔmelA ΔwcaJ ΔmdoH |
| 3FL-S1 | 3-FL | +sorbitol | MDO: PglpF-CA¹, 3xPglpF-futA³, PglpF-marc⁴, Δlacl⁵, |
| | | ↓sucrose | pMK-PmglB_70UTR_SD4- LamB- BiTF-641² |
| LNnT-S2 | LNnT | +Sucrose | MDO 2xPglpF-IgtA⁶, 2xPglpF-galT⁷, PglpF_SD1-Pscr- |
| | | ↓sorbitol | scrYA¹⁰, Pscr-scrBR¹⁰, 2xPglpF-Edic1⁸, Δlacl⁵ ΔsrlA⁹, |
| | | | 1xPglpF-lacY¹¹, ΔglpR¹², pMK-RQ-KanR¹³ |

¹CA = extra colanic acid gene cluster (gmd-wcaG-wcaH-wcal-manC-manB, SEQ ID NO: 109) under the control of a PglpF promoter at a locus that is different than the native locus.
²pMK- PmglB_70UTR_SD4- LamB- BiTF-641- pMK plasmid containing pBR322 as origin of replication and kanamycin as selection marker. The plasmid further contained the transfucosidase, BiTF-641 (SEQ ID NO: 30) with signal peptide LamB (SEQ ID NO: 117) at the N-terminal end. The signal peptide-transglycosidase construct was under control of the PmglB_70UTR_SD4 promoter (SEQ ID NO: 126).
³futA - three independent genomic copies of the gene encoding α-1,3-fucosyl-transferase of SEQ ID NO: 90 under control of the PglpF promoter (SEQ ID NO: 93).
⁴marc gene coding for a heterologous major facilitator superfamily (MFS) transporter of SEQ ID NO: 90.
⁵Δlacl - Deletion of lac repressor makes use of IPTG obsolete.
⁶lgtA - three genomically inserted copies of a gene encoding β-1,3-N-acetyl-glucosaminyltransferase (SEQ ID NO: 94) under control of a PglpF promoter (SEQ ID NO: 93).
⁷galT - one genomically inserted gene encoding β-1,4-Galactosyltransferase (SEQ ID NO: 105) under control of a PglpF promoter (SEQ ID NO: 93).
⁸Edic1 - genomically integrated MFS transporter (SEQ ID NO: 124) under control of the PglpF promoter (SEQ ID NO_93)
⁹ΔsrlA - Deletion of the sorbitol PTS complex IICB^{slr} - to limit growth on sorbitol.
¹⁰scrYA, scrBR two operons encoding the sequences of SEQ ID NO: 97 and 98 and SEQ ID NO: 99 and 100 respectively, and under the control of the PglpF_SD1 promoter (SEQ ID NO: 101) and Pscr promoter (SEQ ID NO: 102), respectively.
¹¹lacY - additional genomically integrated copy of lacY (SEQ ID NO: 1) under control of a PglpF promoter (SEQ ID NO: 93).
¹²ΔglpR - Deletion of glpR repressor.
¹³pMK-RQ-KanR - a plasmid bearing kanamycin resistance marker and having the col/E1 origin of replication to allow the strain to be co-cultured in the presence of kanamycin.

### Fermentation

The co-fermentation with the 3′FL-S1 and LNnT-S2 (genotypes in table 38) was conducted in a 2 L fermenter (Sartorius Biostat B) using essentially the fermentation conditions described in example 9, with the difference that that the initial batch medium was 700 ml and contained 3 g/L glucose, 12 g/L sorbitol and 15 g/L sucrose. Furthermore, no enzyme addition was made during the fermentation. The fermentation was started by inoculating the 3FL-S1 sorbitol strain first to allow it to pre-grow on glucose to adjust for the slower growth rate compared to the LNT-S2-sucrose strain. After the sorbitol strain had consumed all glucose as observed by a drop in CO₂ in the off gas, the sucrose strain was inoculated in a 1:20 ratio compared to the initial sorbitol strain inoculum. After 34 hours, all the initial sorbitol and sucrose had been consumed and the pH had started to rise, at which point the feed was initiated. The sucrose strain grew in a carbon limited manner on sucrose and efficiently produced LNnT, whereas the sorbitol strain did not produce any significant amount of 3FL before 70 hours. This resulted in a somewhat imbalanced mixture of LNnT and 3FL, with excess of the acceptor, LNnT over 3FL.

### Results

Table 39 below shows the process progress in weight % relative to the total weight of substrates and products involved in the transfucosidase reaction occurring in the culture medium.

TABLE 39
Weight % relative to the total weight of substrates
and products as measured during the fermentation.

| Fermentation time | Lactose × H₂O | LNnT | 3FL | LNFP-III |
|---|---|---|---|---|
| Age [h] | wt % | wt % | wt % | wt % |
| 47 | 76.7 | 18.7 | 4.2 | 0.45 |
| 70 | 34.4 | 55.4 | 5.5 | 4.6 |
| 90 | 8.8 | 75.3 | 5.4 | 10.5 |

As can be seen from table 39, LNFP-III was generated in the fermentation broth after 70 h of fermentation, showing that despite the delay in 3FL formation it was possible to provide functional expression and export of the transfucosidase from the 3FL strain. This clearly confirmed that it was feasible to conduct a two-strain hybrid fermentation system with in situ enzyme expression.

The production of LNFP-III using this process could be optimized by for example by adjusting the sorbitol and sucrose feed rates to obtain an earlier production of 3FL and the enzyme, and balance the LNnT production with the 3FL production, or by having a larger initial batch phase on sorbitol to increase the biomass of the 3FL strain sufficiently to make it able to metabolize the sorbitol at the rate it was supplied via the feed. Alternatively, a different carbon source than sorbitol could be considered for the 3FL strain such that the strain can grow at a higher rate and can better match the growth of the sucrose strain.

1. A method for producing one or more oligosaccharides having at least three monosaccharide units, comprising the steps of co-culturing a first and a second genetically modified microbial cell in a culture medium, wherein,
a) the first genetically modified microbial cell produces, under fermentative conditions, a disaccharide or a first oligosaccharide of at least three monosaccharide units and wherein the genetically modified cell
i) is capable of growing on a first carbon source while showing limited or no growth on a second carbon source, and
ii) comprises one or more recombinant nucleic acid sequences encoding at least one glycosyltransferase; and
iii) comprises at least one pathway to produce an activated sugar nucleotide from the first carbon source; and
b) the second genetically modified microbial cell produces, under fermentative conditions, a second oligosaccharide of at least three monosaccharide units and wherein the genetically modified cell
i) is capable of growing on the second carbon source while showing limited or no growth on the first carbon source, and
ii) comprises one or more recombinant nucleic acid sequences encoding at least one glycosyltransferase, and
iii) comprises a biosynthetic pathway to produce an activated sugar nucleotide from the second carbon source.

2. The method according to claim 1, wherein the first carbon source is selected from the group consisting of glucose, glycerol, sucrose, fructose, sorbitol, arabinose, galactose and maltose; and the second carbon source is selected from sucrose, glycerol, fructose, sorbitol, arabinose, galactose, maltose and glucose, and wherein the first and second carbon source are different.

3. The method according to claim 1, wherein the first carbon source is sucrose and the first cell further comprises one or more nucleic acid sequences encoding a PTS-dependent sucrose utilization system or a nucleic acid encoding a sucrose invertase or sucrose hydrolase, or the second carbon source is sucrose and the second cell comprises one or more nucleic acid sequences encoding a PTS-dependent sucrose utilization system or a nucleic acid encoding a sucrose invertase or a sucrose hydrolase.

4. The method according to claim 3, wherein the PTS-dependent sucrose utilization system is encoded by scrY, scrA, scrB and optionally scrR or by the cscABKR-gene cluster and the sucrose invertase is encoded by SacC_Agal or Bff.

5. The method according to claim 3, wherein the first or the second cell has reduced or no ability to grow on glucose or glycerol.

6. The method according to claim 2, wherein the first carbon source is glucose the first cell further comprises one or more nucleic acids encoding one or more glucose transport systems; or the second carbon source is glucose and the second cell further comprises one or more nucleic acids encoding one or more glucose transport systems.

7. The method according to claim 6, wherein the first or the second cell has reduced or no ability to grow on sucrose or glycerol.

8. The method according to claim 1, wherein the one or more recombinant nucleic acidy encoding at least one glycosyltransferase in the first and second genetically modified microbial cell independently is selected from the group consisting of β-1,3-N-acetyl-glucosaminyltransferase, beta-1,3-galactosyltransferase, beta-1,4-galactosyltransferase, alpha-1,2-fucosyltransferase, alpha-1,3-fucosyltransferase, alpha-1,3/4-fucosyltransferase, alpha-1,4-fucosyltransferase, alpha-2,3-sialyltransferase, and alpha-2,6-sialyltransferase.

9. The method according to claim 1, wherein the first and second genetically modified microbial cell are capable of independently producing one or more disaccharides or oligosaccharides selected from the group consisting of LNB, LacNAc, 2′FL, 3FL, 2′FLacNAc, 2′FLNB, Lewis A, Lewis X, 3′SL, 6′SL, 3′SLacNAc, 3′SLNB, sialyl-Lewis A, sialyl-Lewis X, DFL, LNT-II, LNT, LNnT, LNFP-I, LNFP-III, LNFP-IV, LNFP-V, LNFP-VI, FSL, LST-a, LST-b, LST-c, LST-d, LNDFH-II and LNDFH-III, DSLNT, pLNH, pLNnH, LNH, LNnH, (D)F-LNH-I, (D)F-LNH-II, (D)F-LNH-III, F-para-LNH-I, DF-para-LNH, DF-para-LNnH, TF-LNH, FLST-b, FLST-a, FLST-c, S-LNH, S-LNnH-I, FS-LNH, FS-LNnH-I, and DS-F-LNH-II.

10. The method according to claim 1, wherein the one or more oligosaccharides having at least three monosaccharide units is one or more human milk oligosaccharides (HMOs).

11. The method according to claim 1, wherein at least the first or second cell further comprises a recombinant nucleic acid sequence encoding a transporter protein capable of exporting the disaccharide or first oligosaccharide product into the extracellular medium.

12. The method according to claim 1, wherein the first cell produces, under fermentative conditions, LacNAc, LNB, Lewis A, Lewis X, 2′FL, 3FL, LNT-II, LNT, LNnT, LNFP-I, LST-c or LST-a as the most abundant disaccharide or oligosaccharide.

13. The method according to claim 1, wherein the second cell comprises at least one nucleic acid sequence encoding a protein or protein complex which is capable of importing the disaccharide or oligosaccharide produced by the first genetically modified microbial cell.

14. (canceled)

15. The method according to claim 1, wherein the second cell comprises
a) one or more recombinant nucleic acid sequences encoding a glycosyltransferase selected from the group consisting of beta-1,3-galactosyltransferase, beta-1,4-galactosyltransferase, alpha-1,2-fucosyltransferase, alpha-1,3-fucosyltransferase, alpha-1,3/4-fucosyltransferase, alpha-1,4-fucosyltransferase, α-2,3-sialyltransferases, and α-2,6-sialyltransferases, and
b) optionally a non-functional or deleted lactose permease, and
c) optionally a recombinant nucleic acid sequence encoding a MFS transporter capable of exporting the oligosaccharide produced by the second genetically modified microbial cell.

16. The method according to claim 13, wherein the second oligosaccharide is selected from the group consisting of Lewis A, Lewis X, sialyl-LacNAc, sialyl-LNB, sialyl-Lewis X, sialyl-Lewis A, Lewis B, Lewis Y, DFL, FSL, LNT, LNnT, LST-a, LST-b, LST-c, LST-d, LNFP-I, LNFP-II, LNFP-III, LNFP-V, LNFP-VI, LNDFH-I, LNDFH-II, LNDFH-III, DSLNT, pLNH, pLNnH, LNH, LNnH, (D)F-LNH-I, (D)F-LNH-II, (D)F-LNH-III, F-para-LNH-I, DF-para-LNH, DF-para-LNnH, TF-LNH, S-LNFP-I (FLST b), S-LNFP-II (FLST a), S-LNH, S-LNnH-I, FS-LNH, FS-LNnH-I, and DS-F-LNH-II; or a mixture thereof.

17. The method according to claim 1, wherein the one or more oligosaccharides are produced from a donor oligosaccharide and an acceptor disaccharide or acceptor oligosaccharide, produced by the first and the second genetically modified cell and the method further comprises the steps of:
c) making an enzyme with transglycosidase activity available in the culture medium, and
d) incubating the first oligosaccharide or disaccharide, with the second oligosaccharide produced in the co-culture with the transglycosidase enzyme in the culture medium to form a third oligosaccharide of at least four monosaccharide units in the culture medium.

18. The method according to claim 17, wherein the transglycosidase enzyme is either added to the culture medium during the cultivation or is expressed from a recombinant nucleic acid in the first or the second of the genetically modified cells or is expressed from a recombinant nucleic acid in a third genetically modified cell available in the culture medium.

19. The method according to claim 18, wherein the first and the second genetically modified microbial cells comprise a lactose importer.

20. The method according to claim 18, wherein the transglycosidase enzyme is selected from the group consisting of α-1,2-tranfucosidase, α-1,3-transfucosidase, α-1,3/4-transfucosidase, α-2,3-transialylase, α-2,6-transsialylase, trans-lacto-N-biosidase, β-N-acetylglucosaminidase and trans-β-galactosidase.

21. The method according to claim 17, wherein the donor oligosaccharide is selected from the group consisting of Lewis A, Lewis X, 2′FL, 3FL, DFL, sialyl-LacNAc, sialyl-LNB, FSL, 3′SL, 6′SL, LNT, LNnT, LNFP-I and LST-a.

22. The method according to claim 17, wherein the acceptor disaccharide or acceptor oligosaccharide is selected from the group consisting LacNAc, LNB, Lewis A, Lewis X, 2′FL, 3FL, LNT-II, LNT, LNnT, Para-LNnH, LNFP-I, LNFP-II, LNFP-III, LNFP-IV, LNFP-V, LNFP-VI, 3′SL, 6′SL, LST-a, and LST-c.

23. The method according to claim 17, wherein the one or more oligosaccharide produced by incubating the transsialidase enzyme with the donor and acceptor molecules has at least three monosaccharide units, such as at least four monosaccharide units, and are selected from the group consisting of DFL, LNFP-I, LNDFH-I, Lewis-Y, Lewis-B, LNFP-II, LNFP-V, DFL, LNFP-III, LNFP-IV, LNDFH-I, LNDFH-II, LNDFH-III, LNDFH-III, LNDFH-II, Lewis-X, Lewis-A, DF-para-LNnH, FLSTa (S-LNFP-II), FSL, LSTa, FLSTa, 3′SLacNAc, 3′SLNB, LSTc, FLSTc, 6′SLN, FLSTb (S-LNFP-I), LSTb, DSLNT, 6′SLNB, 6′SLacNAc, para-LNH, gal-LNnT, LNFP-V, F-p-LNH, S-p-LNH, GlcNAc(1-3)-3FL, Sialyl-Lewis X, Sialyl-Lewis A.

24. (canceled)

25. The method according to claim 1, wherein the first and the second cell are independently selected from the group consisting of Escherichia coli, Corynebacterium glutamicum, Lactococcus lactis, Bacillus subtilis, Streptomyces lividans, Pichia pastoris and Saccharomyces cerevisiae.

26. The method according to claim 25, wherein the first and the second cell are the same species.

27. The method according to claim 26, wherein the first and second cell is Escherichia coli.

28. (canceled)
